(19) United States
(12) Patent Application Publication
(10) Pub. No.: US 2026/0123276 A1
(43) Pub. Date: Apr. 30, 2026

(54) COMPOUND, LIGHT-EMITTING DEVICE INCLUDING SAME, AND ELECTRONIC APPARATUS AND ELECTRONIC EQUIPMENT EACH INCLUDING SAME

(71) Applicant: Samsung Display Co., Ltd., Yongin-si (KR)

(72) Inventors: Gyeongheon Kim, Yongin-si (KR); Junyoung Lee, Yongin-si (KR); Hoilim Kim, Yongin-si (KR); Hayoung Yu, Yongin-si (KR)

(21) Appl. No.: 19/209,625

(22) Filed: May 15, 2025

(30) Foreign Application Priority Data
Oct. 31, 2024 (KR) 10-2024-0152966

(51) Int. Cl. H10K 85/60; C07C 255/47; C07C 255/52; C09K 11/06; H10K 50/12; H10K 50/15; H10K 85/30; H10K 101/00

(52) U.S. Cl. CPC H10K 85/623; C07C 255/47; C07C 255/52; C09K 11/06; H10K 85/346; C07C 2603/52; C09K 2211/1007; C09K 2211/1014; C09K 2211/1022; C09K 2211/1029; C09K 2211/1044; C09K 2211/185; H10K 50/12; H10K 50/156; H10K 2101/90

(57) ABSTRACT

A light-emitting device includes a first electrode, a second electrode opposite to the first electrode, and an interlayer between the first electrode and the second electrode and including an emission layer and a compound represented by Formula 1. In addition, there are provided an electronic apparatus and electronic equipment each including the light-emitting device, and the compound represented by Formula 1.

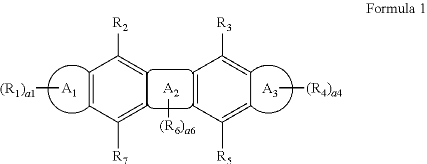

### CROSS-REFERENCE TO RELATED APPLICATION

The present application claims priority to and the benefit of Korean Patent Application No. 10-2024-0152966, filed on Oct. 31, 2024, in the Korean Intellectual Property Office, the entire disclosure of which is incorporated herein by reference.

### BACKGROUND

### 1. Field

One or more embodiments of the present disclosure relate to a compound, a light-emitting device including the same, and an electronic apparatus including the light-emitting device.

### 2. Description of the Related Art

Among light-emitting devices, self-emissive devices (e.g., organic light-emitting devices) are notable for their relatively wide viewing angles, high contrast ratios, short response times, and/or enhanced (e.g., excellent, desirable, and/or suitable) characteristics in terms of luminance, driving voltage, and/or response speed.

In a light-emitting device, a first electrode is arranged on a substrate, followed sequentially by a hole transport region, an emission layer, an electron transport region, and a second electrode. Holes provided from the first electrode move toward the emission layer through the hole transport region, while electrons provided from the second electrode move toward the emission layer through the electron transport region. These carriers, namely the holes and electrons, recombine in the emission layer to produce excitons. These excitons transition and decay from an excited state to a ground state to thereby generate light.

### SUMMARY

One or more aspects of embodiments of the present disclosure are directed toward a compound and a light-emitting device including the compound.

Additional aspects will be set forth in part in the description which follows and, in part, will be apparent from the description, or may be learned by practice of the presented embodiments of the disclosure.

According to one or more embodiments of the present disclosure,

A compound represented by Formula 1 is provided:

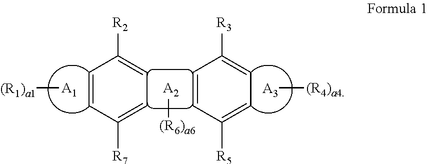

According to one or more embodiments of the present disclosure, a light-emitting device including the compound represented by Formula 1 is provided.

According to one or more embodiments of the present disclosure, an electronic apparatus including the light-emitting device is provided.

### BRIEF DESCRIPTION OF DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIGS. 1 and 2 are each a schematic view of a light-emitting device according to one or more embodiments of the present disclosure;

FIG. 3 is a cross-sectional view of an electronic apparatus according to one or more embodiments of the present disclosure;

FIG. 4 is a cross-sectional view of an electronic apparatus according to one or more embodiments of the present disclosure;

FIG. 5 is a schematic view of an electronic equipment including a light-emitting device according to one or more embodiments of the present disclosure;

FIG. 6 is a schematic view of an exterior of a vehicle as electronic equipment including a light-emitting device according to one or more embodiments of the present disclosure; and

FIGS. 7A-7C are each a schematic view of an interior of a vehicle according to one or more embodiments of the present disclosure.

### DETAILED DESCRIPTION

Reference will now be made in more detail to one or more embodiments, examples of which are illustrated in the accompanying drawings, wherein like reference numerals refer to like elements throughout the disclosure, and duplicative descriptions thereof may not be provided for conciseness. In this regard, the presented embodiments may have different forms and should not be construed as being limited to the descriptions set forth herein. Accordingly, one or more embodiments are merely described in more detail, by referring to the drawings, to explain aspects of the present disclosure. As used herein, the term “and/or” or “or” may include any and all combinations of one or more of the associated listed items. Throughout the disclosure, the expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

The efficiency, operation, and lifetime characteristics of light-emitting devices need to be improved continuously and desirably, and for this purpose, hole injection layer materials used in the hole transport region have been progressively researched and developed. The performance of a light-emitting device is improved by a p-dopant material of the present disclosure used in the hole injection layer.

According to one or more embodiments of the present disclosure, a compound may be represented by Formula 1:

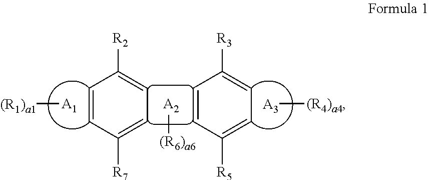

wherein, in Formula 1,
A₁ and A₃ may each independently be hydrogen, deuterium, a cyano group, or a C₅ carbocyclic group,
A₂ may be a C₄-C₆ carbocyclic group,
R₁ to R₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₈-C₆₀ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a4, and a6 are independently integers from 0 to 3, and if (e.g., when) a1 is 2 or greater, each R₁ may be identical to or different from each other, if (e.g., when) a4 is 2 or greater, each R₄ may be identical to or different from each other, and if (e.g., when) a6 is 2 or greater, each R₆ may be identical to or different from each other,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof,
Formula 1 may include a cyano group.

The wording “A₁ and A₃ may each independently be a C₅ carbocyclic group” refers to the embodiment in which A₁ as a C₅ carbocyclic group and A₃ as a C₅ carbocyclic group may each independently be fused to a benzene moiety of Formula 1.

A₁ may be a portion of Formula 1 excluding benzene moieties, A₂, A₃, and other substituents. For example, A₁ may be [symbol] . As may be a portion of Formula 1 excluding benzene moieties, A₁, A₂, and other substituents. For example, A₃ may be [symbol] .

For example, in one or more embodiments, hydrogen, deuterium, or a cyano group may be bonded to the carbon positions of the benzene moieties at which A₁ and A₃ may be fused.

The wording “A₂ may be a C₄-C₆ carbocyclic group” refers to the embodiment in which A₂ as a C₄-C₆ carbocyclic group may be fused to the two benzene moieties of Formula 1.

A₂ may be a portion of Formula 1 excluding two benzene moieties, A₁, A₃, and other substituents. For example, A₂ may be [symbol] .

That Formula 1 includes a cyano group may refer to that at least one cyano group is bonded to any position (e.g., any suitable position) in Formula 1.

Definitions of the C₅ carbocyclic group and the C₄-C₆ carbocyclic group may be referred to the definition of the C₃-C₆₀ carbocyclic group described herein.

According to one or more embodiments, A₁ and Aₛ may each independently be hydrogen, deuterium, a cyano group, or
a cyclopentene moiety.

For example, in one or more embodiments, A₁ and Aₛ may each be a cyclopentene moiety.

According to one or more embodiments, A₂ may be a cyclobutane moiety or a bicyclo[2,2,0]hex-1(4)-ene moiety.

For example, [symbol] may be a cyclobutane moiety or a bicyclo[2,2,0]hex-1(4)-ene moiety.

According to one or more embodiments, the compound represented by Formula 1 may be a symmetrical compound. The term “symmetrical compound” as used herein refers to a compound whose chemical structure has a symmetry element (e.g., a line, a plane, or a point).

For example, in one or more embodiments, the compound of Formula 1 may be a point-symmetric compound or a line-symmetric compound.

According to one or more embodiments, the compound represented by Formula 1 may be represented by Formula 1-1:

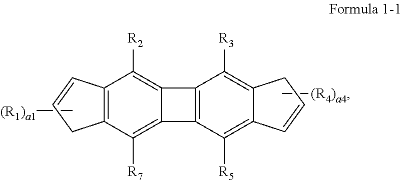

in Formula 1-1,
The definitions of R₁ to R₅ and R₇, a1 and a4 may be same as defined in Formula 1, and Formula 1-1 may include a cyano group.

According to one or more embodiments, the compound represented Formula 1 may be represented by Formula 1-2:

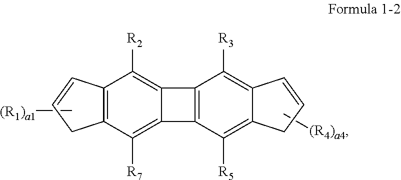

in Formula 1-2,

The definitions of R₁ to R₅ and R₇, a1 and a4 may be same as defined in Formula 1, and Formula 1-2 may include a cyano group.

According to one or more embodiments, the compound represented by Formula 1 may be represented by Formula 1-3:

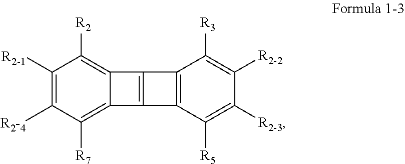

in Formula 1-3,
R₂₋₁ to R₂₋₄ may each independently be hydrogen, deuterium, or a cyano group, and the definitions of R₂, R₃, R₅, and R₇ may be same as defined in Formula 1, and Formula 1-3 may include a cyano group.

According to one or more embodiments, the compound represented by Formula 1 may be represented by Formula 1-4:

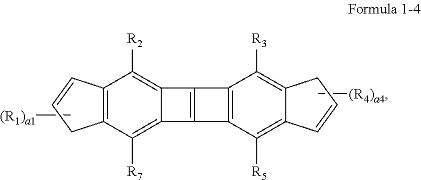

in Formula 1-4,
The definitions of R₁ to R₅ and R₇, a1 and a4 may be same as defined in Formula 1, and Formula 1-4 may include a cyano group.

According to one or more embodiments, the compound represented by Formula 1 may be represented by Formula 1-5:

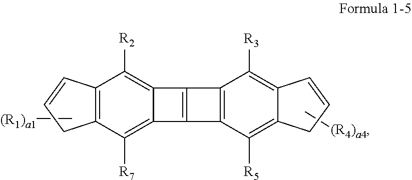

in Formula 1-5,
The definitions of R₁ to R₅ and R₇, a1 and a4 may be same as defined in Formula 1, and Formula 1-5 may include a cyano group.

According to one or more embodiments, the compound represented by the Formula 1 may include any one selected from among the following compounds:

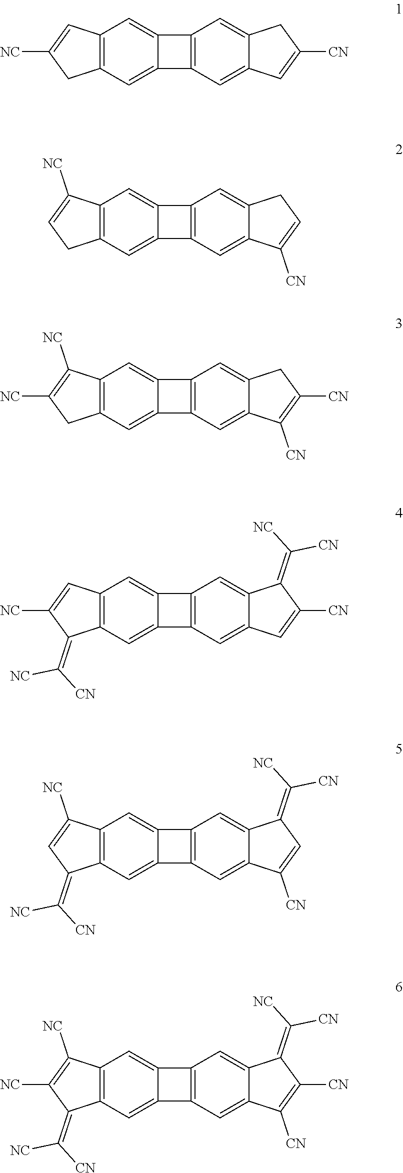 [SMILES: N#CC1=Cc2cc3c(cc2C1)-c1cc2c(cc1-3)CC(C#N)=C2 ; N#CC1=CCc2cc3c(cc21)-c1cc2c(cc1-3)C(C#N)=CC2 ; N#CC1=C(C#N)c2cc3c(cc2C1)-c1cc2c(cc1-3)CC(C#N)=C2C#N ; N#CC(C#N)=c1c(C#N)cc2cc3c(cc12)c1cc2cc(C#N)c(=C(C#N)C#N)c2cc31 ; N#CC(C#N)=c1cc(C#N)c2cc3c(cc12)c1cc2c(C#N)cc(=C(C#N)C#N)c2cc31 ; N#CC(C#N)=c1c(C#N)c(C#N)c2cc3c(cc12)c1cc2c(C#N)c(C#N)c(=C(C#N)C#N)c2cc31]

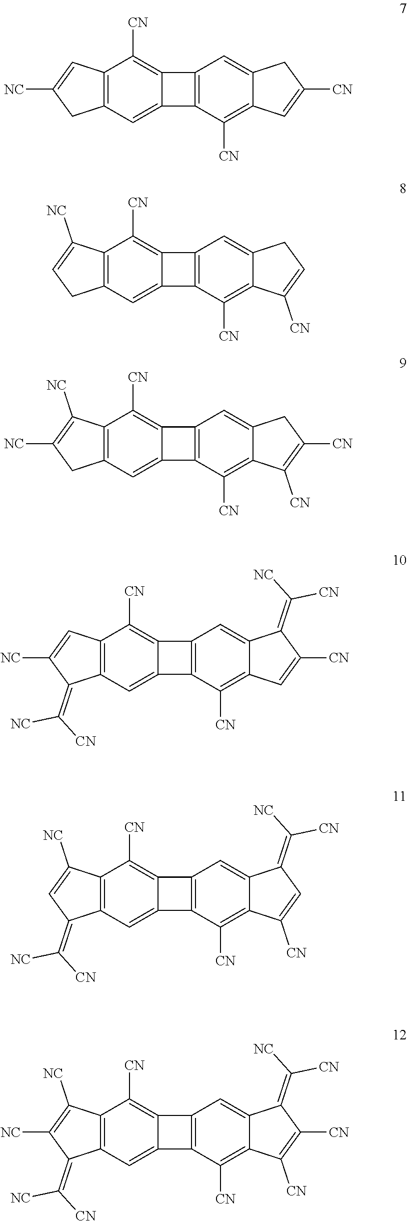 [SMILES: N#CC1=Cc2c(cc3c(c2C#N)-c2cc4c(c(C#N)c2-3)C=C(C#N)C4)C1 ; N#CC1=CCc2cc3c(c(C#N)c21)-c1cc2c(c(C#N)c1-3)C(C#N)=CC2 ; N#CC1=C(C#N)c2c(cc3c(c2C#N)-c2cc4c(c(C#N)c2-3)C(C#N)=C(C#N)C4)C1 ; N#CC(C#N)=c1c(C#N)cc2c(C#N)c3c(cc12)c1c(C#N)c2cc(C#N)c(=C(C#N)C#N)c2cc31 ; N#CC(C#N)=c1cc(C#N)c2c(C#N)c3c(cc12)c1c(C#N)c2c(C#N)cc(=C(C#N)C#N)c2cc31 ; N#CC(C#N)=c1c(C#N)c(C#N)c2c(C#N)c3c(cc12)c1c(C#N)c2c(C#N)c(C#N)c(=C(C#N)C#N)c2cc31]

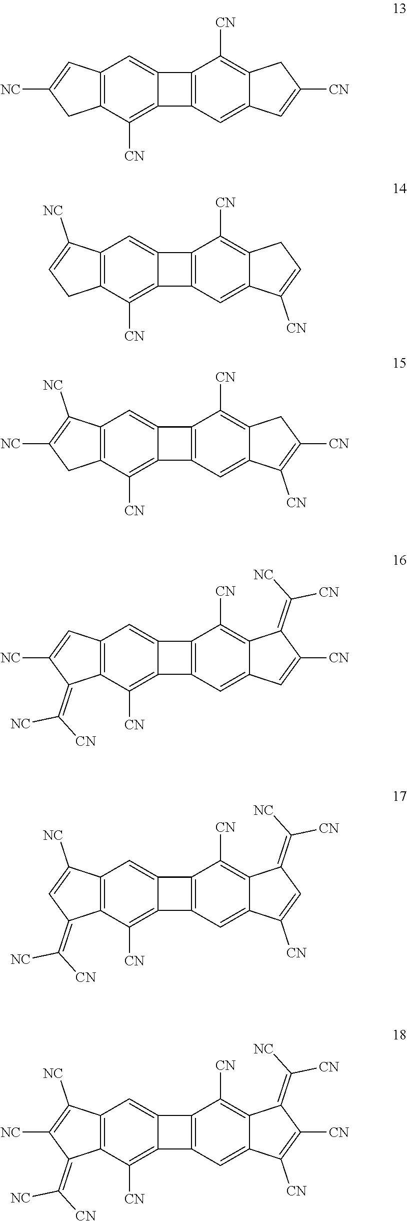 [SMILES: N#CC1=Cc2cc3c(c(C#N)c2C1)-c1cc2c(c(C#N)c1-3)CC(C#N)=C2 ; N#CC1=CCc2c1cc1c(c2C#N)-c2cc3c(c(C#N)c2-1)CC=C3C#N ; N#CC1=C(C#N)c2cc3c(c(C#N)c2C1)-c1cc2c(c(C#N)c1-3)CC(C#N)=C2C#N ; N#CC(C#N)=c1c(C#N)cc2cc3c(c(C#N)c12)c1cc2cc(C#N)c(=C(C#N)C#N)c2c(C#N)c31 ; N#CC(C#N)=c1cc(C#N)c2cc3c(c(C#N)c12)c1cc2c(C#N)cc(=C(C#N)C#N)c2c(C#N)c31 ; N#CC(C#N)=c1c(C#N)c(C#N)c2cc3c(c(C#N)c12)c1cc2c(C#N)c(C#N)c(=C(C#N)C#N)c2c(C#N)c31]

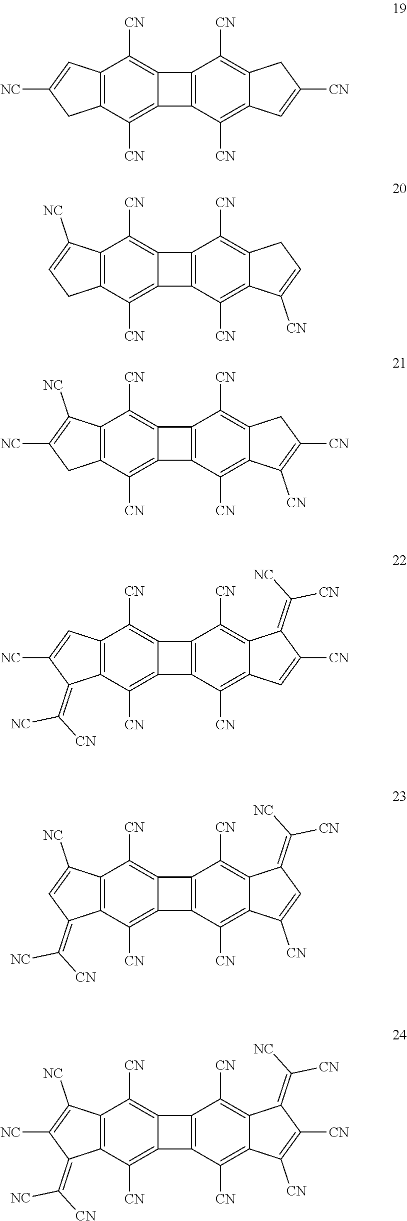

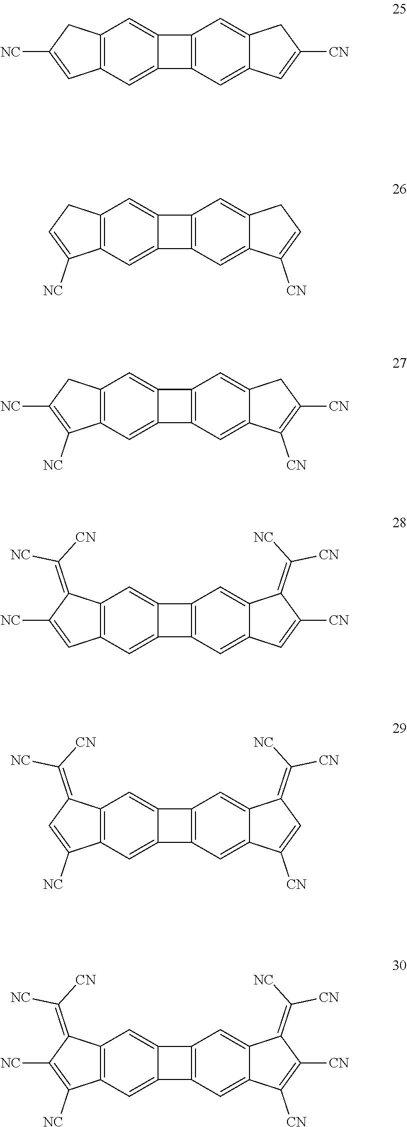 [SMILES: N#CC1=Cc2cc3c(cc2C1)-c1cc2c(cc1-3)C=C(C#N)C2 ; N#CC1=CCc2cc3c(cc21)-c1cc2c(cc1-3)CC=C2C#N ; N#CC1=C(C#N)c2cc3c(cc2C1)-c1cc2c(cc1-3)C(C#N)=C(C#N)C2 ; N#CC(C#N)=c1c(C#N)cc2cc3c4cc5cc(C#N)c(=C(C#N)C#N)c5cc4c3cc12 ; N#CC(C#N)=c1cc(C#N)c2cc3c4cc5c(C#N)cc(=C(C#N)C#N)c5cc4c3cc12 ; N#CC(C#N)=c1c(C#N)c(C#N)c2cc3c4cc5c(C#N)c(C#N)c(=C(C#N)C#N)c5cc4c3cc12]

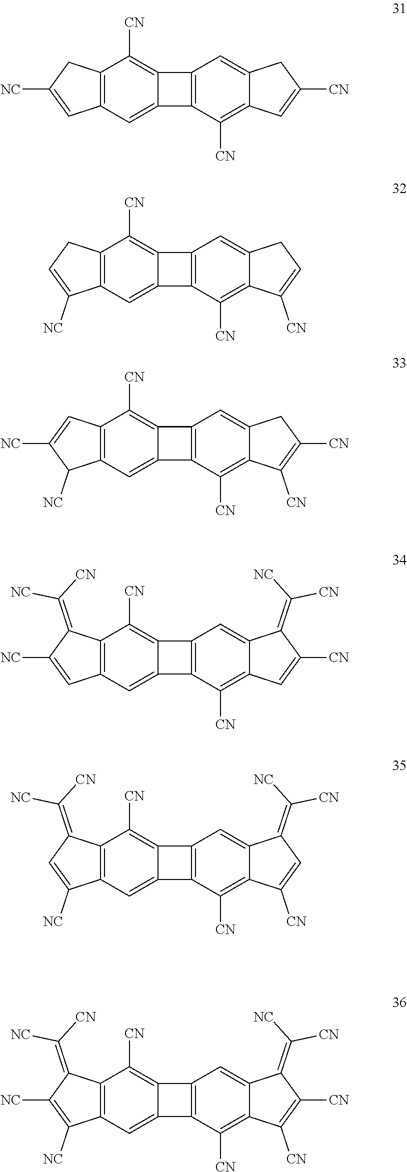 [SMILES: N#CC1=Cc2c(cc3c(c2C#N)-c2cc4c(c(C#N)c2-3)CC(C#N)=C4)C1 ; N#CC1=CCc2c1cc1c(c2C#N)-c2cc3c(c(C#N)c2-1)C(C#N)=CC3 ; N#CC1=Cc2c(cc3c(c2C#N)-c2cc4c(c(C#N)c2-3)C(C#N)=C(C#N)C4)C1C#N ; N#CC(C#N)=c1c(C#N)cc2c(C#N)c3c4cc5cc(C#N)c(=C(C#N)C#N)c5c(C#N)c4c3cc12 ; N#CC(C#N)=c1cc(C#N)c2c(C#N)c3c4cc5c(C#N)cc(=C(C#N)C#N)c5c(C#N)c4c3cc12 ; N#CC(C#N)=c1c(C#N)c(C#N)c2c(C#N)c3c4cc5c(C#N)c(C#N)c(=C(C#N)C#N)c5c(C#N)c4c3cc12]

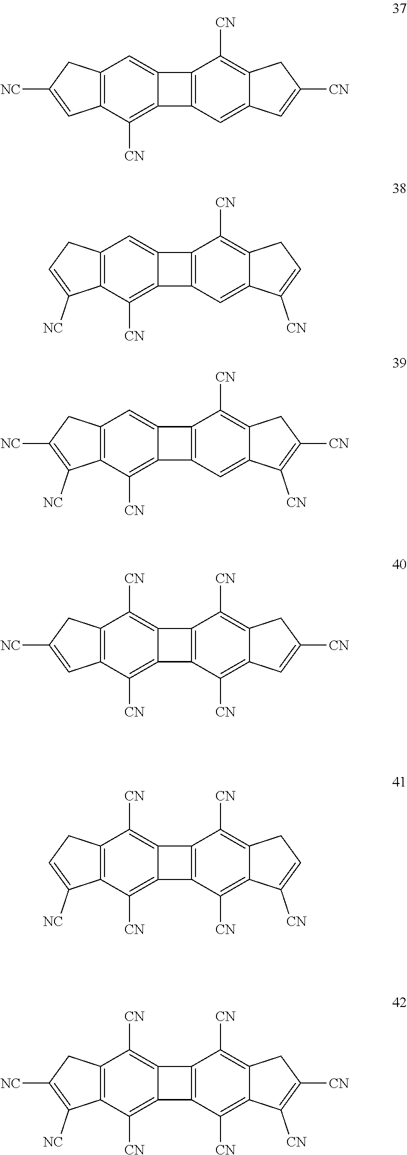 [SMILES: N#CC1=Cc2c(cc3c(c2C#N)-c2cc4c(c(C#N)c2-3)CC(C#N)=C4)C1 ; N#CC1=CCc2c1cc1c(c2C#N)-c2cc3c(c(C#N)c2-1)C(C#N)=CC3 ; N#CC1=C(C#N)c2cc3c(c(C#N)c2C1)-c1cc2c(c(C#N)c1-3)C(C#N)=C(C#N)C2 ; N#CC1=Cc2c(C#N)c3c(c(C#N)c2C1)-c1c(C#N)c2c(c(C#N)c1-3)C=C(C#N)C2 ; N#CC1=CCc2c(C#N)c3c(c(C#N)c21)-c1c(C#N)c2c(c(C#N)c1-3)CC=C2C#N ; N#CC1=C(C#N)c2c(C#N)c3c(c(C#N)c2C1)-c1c(C#N)c2c(c(C#N)c1-3)C(C#N)=C(C#N)C2]

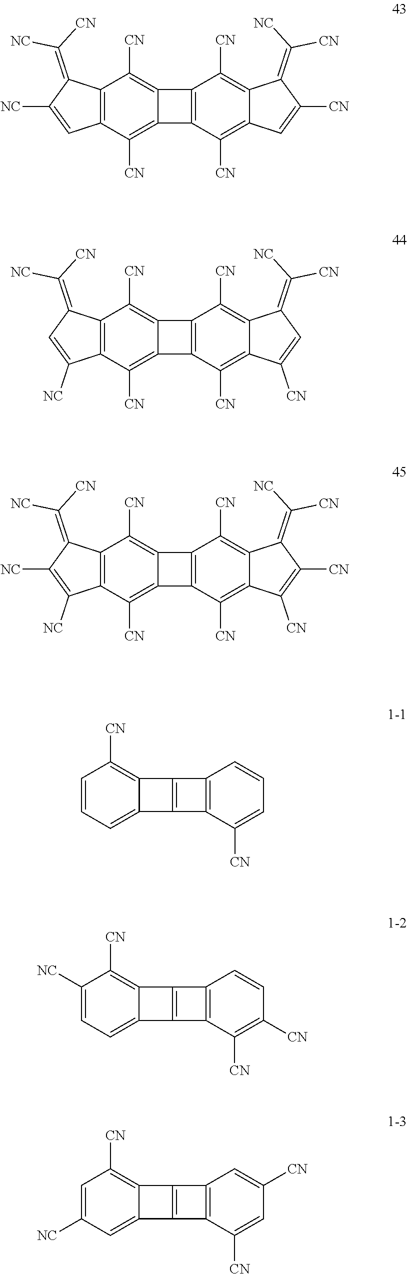 [SMILES: N#CC(C#N)=c1c(C#N)cc2c(C#N)c3c4c(C#N)c5cc(C#N)c(=C(C#N)C#N)c5c(C#N)c4c3c(C#N)c12 ; N#CC(C#N)=c1cc(C#N)c2c(C#N)c3c4c(C#N)c5c(C#N)cc(=C(C#N)C#N)c5c(C#N)c4c3c(C#N)c12 ; N#CC(C#N)=c1c(C#N)c(C#N)c2c(C#N)c3c4c(C#N)c5c(C#N)c(C#N)c(=C(C#N)C#N)c5c(C#N)c4c3c(C#N)c12 ; N#Cc1cccc2c3c4c(C#N)cccc4c=3c12 ; N#Cc1ccc2c3c4c(C#N)c(C#N)ccc4c=3c2c1C#N ; N#Cc1cc(C#N)c2c3c4cc(C#N)cc(C#N)c4c=3c2c1]

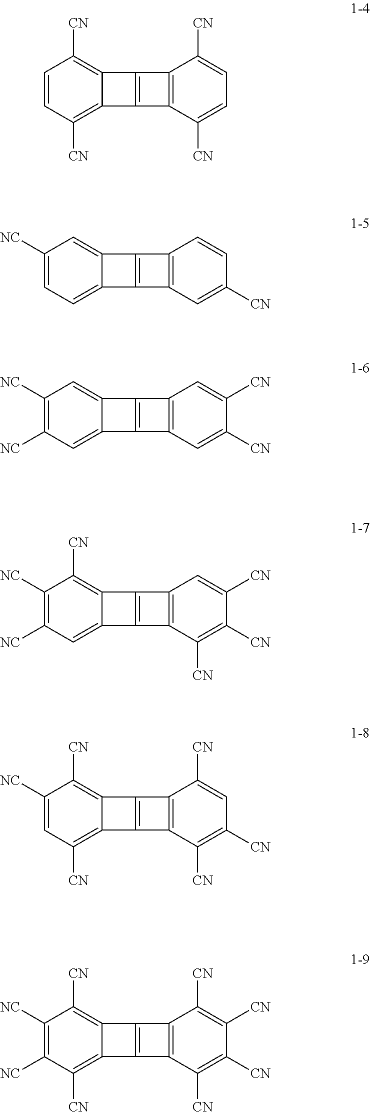 [SMILES: N#Cc1ccc(C#N)c2c3c4c(C#N)ccc(C#N)c4c=3c12 ; N#Cc1ccc2c3c4cc(C#N)ccc4c=3c2c1 ; N#Cc1cc2c3c4cc(C#N)c(C#N)cc4c=3c2cc1C#N ; N#Cc1cc2c3c4c(C#N)c(C#N)c(C#N)cc4c=3c2c(C#N)c1C#N ; N#Cc1cc(C#N)c2c3c4c(C#N)c(C#N)cc(C#N)c4c=3c2c1C#N ; N#Cc1c(C#N)c(C#N)c2c3c4c(C#N)c(C#N)c(C#N)c(C#N)c4c=3c2c1C#N]

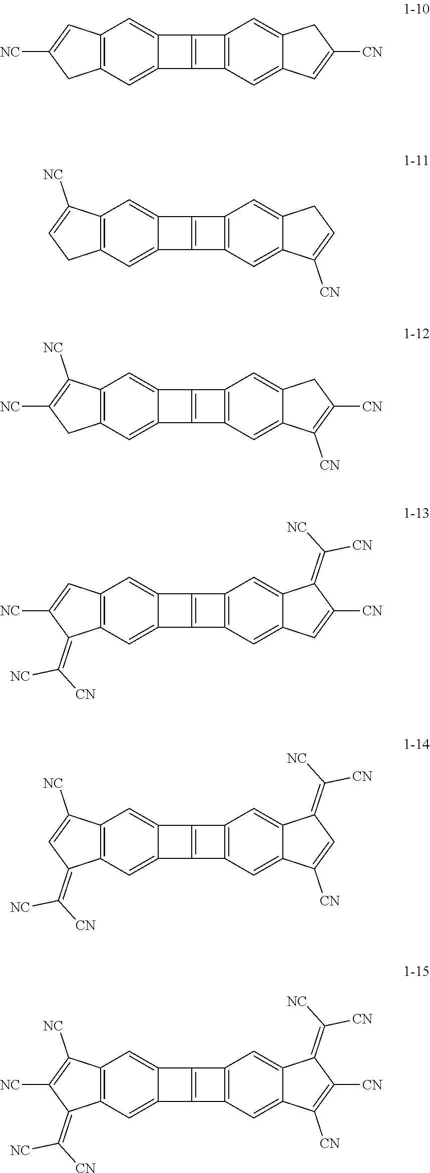 [SMILES: N#CC1=Cc2cc3c4c5cc6c(cc5c=4c3cc2C1)C=C(C#N)C6 ; N#CC1=CCc2cc3c4c5cc6c(cc5c=4c3cc21)CC=C6C#N ; N#CC1=C(C#N)c2cc3c4c5cc6c(cc5c=4c3cc2C1)C(C#N)=C(C#N)C6 ; N#CC1=Cc2cc3c4c5cc6c(cc5c=4c3cc2C1=C(C#N)C#N)C=C(C#N)C6=C(C#N)C#N ; N#CC1=CC(=C(C#N)C#N)c2cc3c4c5cc6c(cc5c=4c3cc21)C(=C(C#N)C#N)C=C6C#N ; N#CC(C#N)=C1C(C#N)=C(C#N)c2cc3c4c5cc6c(cc5c=4c3cc21)C(C#N)=C(C#N)C6=C(C#N)C#N]

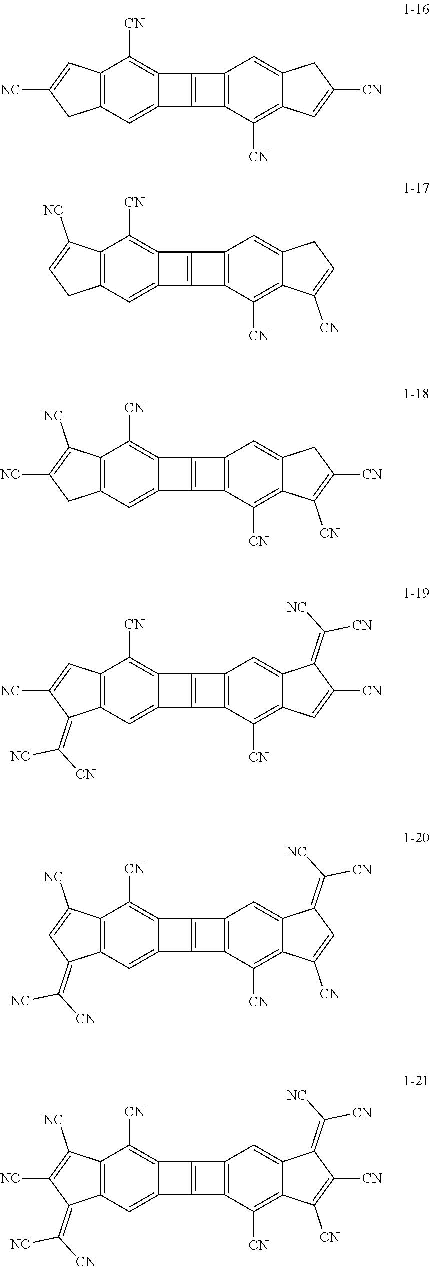 [SMILES: N#CC1=Cc2c(cc3c4c5c(C#N)c6c(cc5c=4c3c2C#N)CC(C#N)=C6)C1 ; N#CC1=CCc2cc3c4c5c(C#N)c6c(cc5c=4c3c(C#N)c21)CC=C6C#N ; N#CC1=C(C#N)c2c(cc3c4c5c(C#N)c6c(cc5c=4c3c2C#N)CC(C#N)=C6C#N)C1 ; N#CC1=Cc2c(cc3c4c5c(C#N)c6c(cc5c=4c3c2C#N)C(=C(C#N)C#N)C(C#N)=C6)C1=C(C#N)C#N ; N#CC1=CC(=C(C#N)C#N)c2cc3c4c5c(C#N)c6c(cc5c=4c3c(C#N)c21)C(=C(C#N)C#N)C=C6C#N ; N#CC(C#N)=C1C(C#N)=C(C#N)c2c1cc1c3c4c(C#N)c5c(cc4c=3c1c2C#N)C(=C(C#N)C#N)C(C#N)=C5C#N]

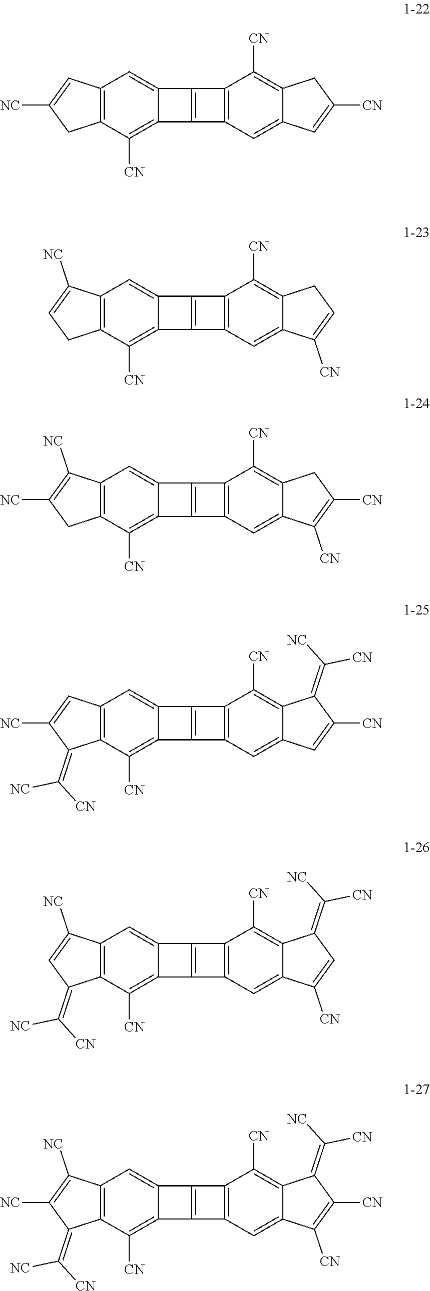 [SMILES: N#CC1=Cc2cc3c4c5c(C#N)c6c(cc5c=4c3c(C#N)c2C1)C=C(C#N)C6 ; N#CC1=CCc2c1cc1c3c4c(C#N)c5c(cc4c=3c1c2C#N)C(C#N)=CC5 ; N#CC1=C(C#N)c2cc3c4c5c(C#N)c6c(cc5c=4c3c(C#N)c2C1)C(C#N)=C(C#N)C6 ; N#CC1=Cc2cc3c4c5c(C#N)c6c(cc5c=4c3c(C#N)c2C1=C(C#N)C#N)C=C(C#N)C6=C(C#N)C#N ; N#CC1=CC(=C(C#N)C#N)c2c1cc1c3c4c(C#N)c5c(cc4c=3c1c2C#N)C(C#N)=CC5=C(C#N)C#N ; N#CC(C#N)=C1C(C#N)=C(C#N)c2cc3c4c5c(C#N)c6c(cc5c=4c3c(C#N)c21)C(C#N)=C(C#N)C6=C(C#N)C#N]

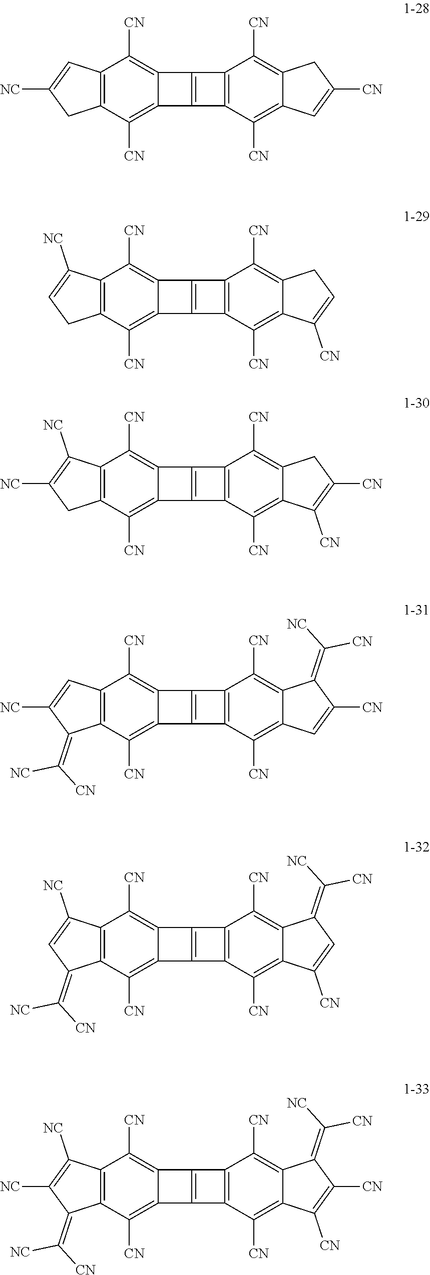 [SMILES: N#CC1=Cc2c(c(C#N)c3c4c5c(C#N)c6c(c(C#N)c5c=4c3c2C#N)CC(C#N)=C6)C1 ; N#CC1=CCc2c1c(C#N)c1c3c4c(C#N)c5c(c(C#N)c4c=3c1c2C#N)C(C#N)=CC5 ; N#CC1=C(C#N)c2c(c(C#N)c3c4c5c(C#N)c6c(c(C#N)c5c=4c3c2C#N)CC(C#N)=C6C#N)C1 ; N#CC1=Cc2c(c(C#N)c3c4c5c(C#N)c6c(c(C#N)c5c=4c3c2C#N)C(=C(C#N)C#N)C(C#N)=C6)C1=C(C#N)C#N ; N#CC1=CC(=C(C#N)C#N)c2c1c(C#N)c1c3c4c(C#N)c5c(c(C#N)c4c=3c1c2C#N)C(C#N)=CC5=C(C#N)C#N ; N#CC(C#N)=C1C(C#N)=C(C#N)c2c1c(C#N)c1c3c4c(C#N)c5c(c(C#N)c4c=3c1c2C#N)C(=C(C#N)C#N)C(C#N)=C5C#N]

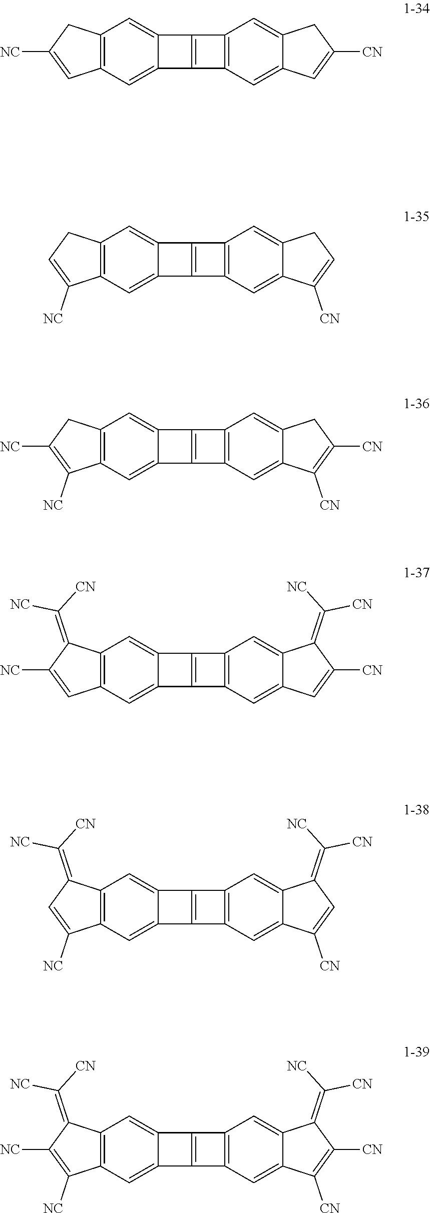 [SMILES: N#CC1=Cc2cc3c4c5cc6c(cc5c=4c3cc2C1)CC(C#N)=C6 ; N#CC1=CCc2cc3c4c5cc6c(cc5c=4c3cc21)C(C#N)=CC6 ; N#CC1=C(C#N)c2cc3c4c5cc6c(cc5c=4c3cc2C1)CC(C#N)=C6C#N ; N#CC1=Cc2cc3c4c5cc6c(cc5c=4c3cc2C1=C(C#N)C#N)C(=C(C#N)C#N)C(C#N)=C6 ; N#CC1=CC(=C(C#N)C#N)c2cc3c4c5cc6c(cc5c=4c3cc21)C(C#N)=CC6=C(C#N)C#N ; N#CC(C#N)=C1C(C#N)=C(C#N)c2cc3c4c5cc6c(cc5c=4c3cc21)C(=C(C#N)C#N)C(C#N)=C6C#N]

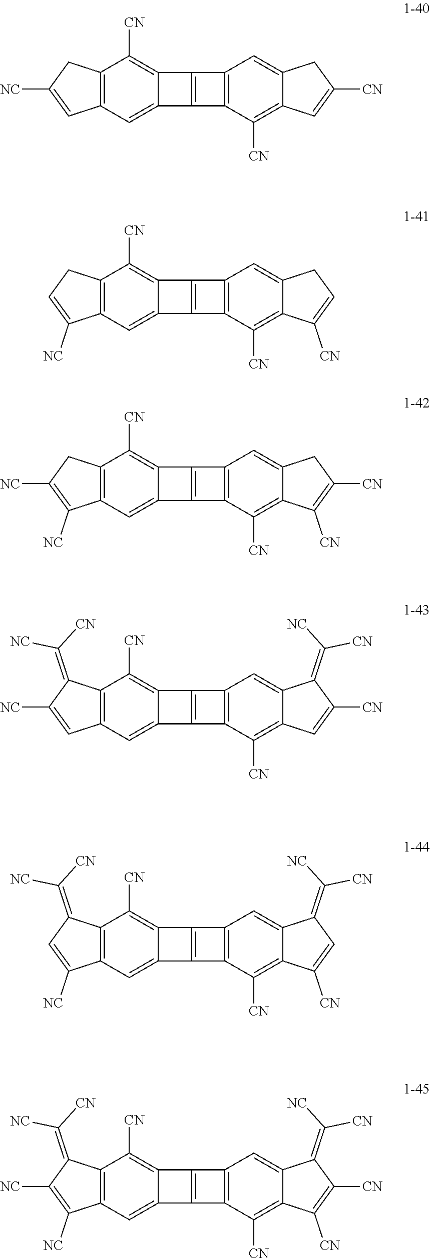 [SMILES: N#CC1=Cc2c(cc3c4c5c(C#N)c6c(cc5c=4c3c2C#N)C=C(C#N)C6)C1 ; N#CC1=CCc2c1cc1c3c4c(C#N)c5c(cc4c=3c1c2C#N)CC=C5C#N ; N#CC1=C(C#N)c2cc3c4c5c(C#N)c6c(cc5c=4c3c(C#N)c2C1)CC(C#N)=C6C#N ; N#CC1=Cc2c(cc3c4c5c(C#N)c6c(cc5c=4c3c2C#N)C=C(C#N)C6=C(C#N)C#N)C1=C(C#N)C#N ; N#CC1=CC(=C(C#N)C#N)c2c1cc1c3c4c(C#N)c5c(cc4c=3c1c2C#N)C(=C(C#N)C#N)C=C5C#N ; N#CC(C#N)=C1C(C#N)=C(C#N)c2c1cc1c3c4c(C#N)c5c(cc4c=3c1c2C#N)C(C#N)=C(C#N)C5=C(C#N)C#N]

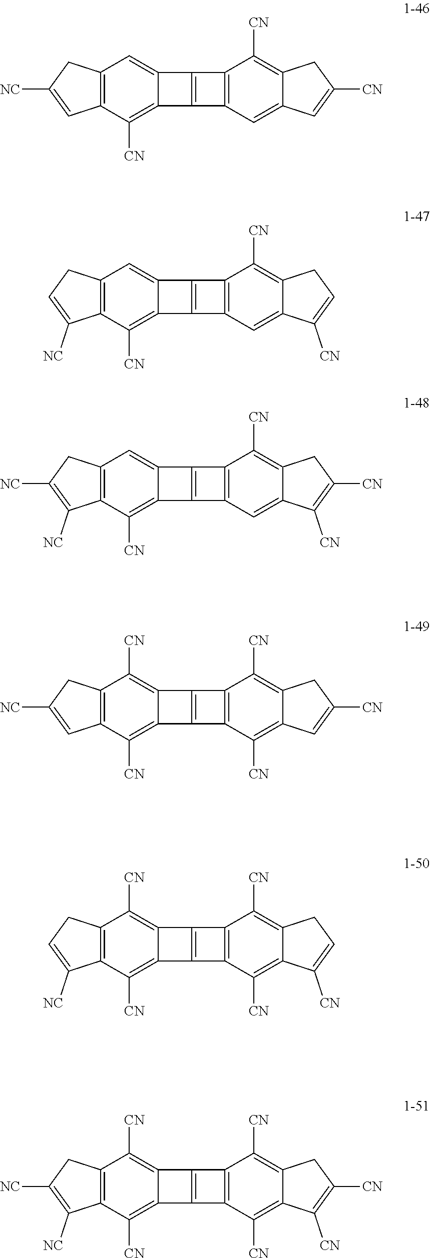 [SMILES: N#CC1=Cc2c(cc3c4c5c(C#N)c6c(cc5c=4c3c2C#N)C=C(C#N)C6)C1 ; N#CC1=CCc2c1cc1c3c4c(C#N)c5c(cc4c=3c1c2C#N)CC=C5C#N ; N#CC1=C(C#N)c2cc3c4c5c(C#N)c6c(cc5c=4c3c(C#N)c2C1)CC(C#N)=C6C#N ; N#CC1=Cc2c(c(C#N)c3c4c5c(C#N)c6c(c(C#N)c5c=4c3c2C#N)C=C(C#N)C6)C1 ; N#CC1=CCc2c1c(C#N)c1c3c4c(C#N)c5c(c(C#N)c4c=3c1c2C#N)CC=C5C#N ; N#CC1=C(C#N)c2c(c(C#N)c3c4c5c(C#N)c6c(c(C#N)c5c=4c3c2C#N)C(C#N)=C(C#N)C6)C1]

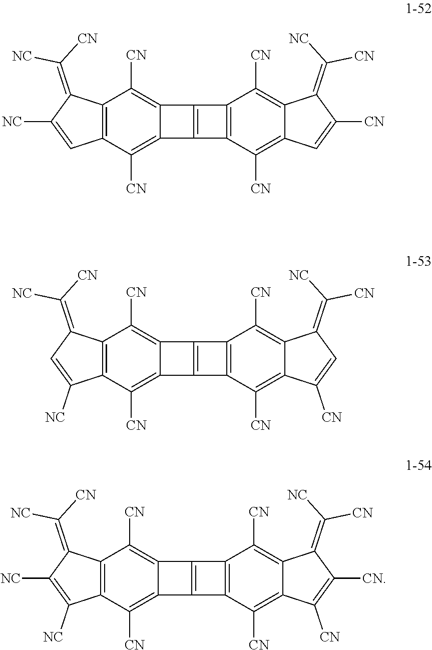 [SMILES: N#CC1=Cc2c(c(C#N)c3c4c5c(C#N)c6c(c(C#N)c5c=4c3c2C#N)C=C(C#N)C6=C(C#N)C#N)C1=C(C#N)C#N ; N#CC1=CC(=C(C#N)C#N)c2c1c(C#N)c1c3c4c(C#N)c5c(c(C#N)c4c=3c1c2C#N)C(=C(C#N)C#N)C=C5C#N ; N#CC(C#N)=C1C(C#N)=C(C#N)c2c1c(C#N)c1c3c4c(C#N)c5c(c(C#N)c4c=3c1c2C#N)C(C#N)=C(C#N)C5=C(C#N)C#N]

According to one or more embodiments of the present disclosure, a light-emitting device includes:
a first electrode;
a second electrode opposite to (e.g., facing) the first electrode; and
an interlayer between (e.g., arranged between) the first electrode and the second electrode and including an emission layer,
wherein the light-emitting device includes the compound of Formula 1, thereby having excellent or suitable driving voltage, efficiency, and lifespan.

According to one or more embodiments, the first electrode may be an anode, the second electrode may be a cathode, and the interlayer may further a hole transport region between (e.g., arranged between) the first electrode and the emission layer and including a hole injection layer; a hole transport layer, an electron blocking layer, a emission auxiliary layer, or any combination thereof; and/or
an electron transport region between (e.g., arranged between) the second electrode and the emission layer and including a hole blocking layer, an electron transport layer, an electron injection layer, or any combination thereof.

According to one or more embodiments, the hole transport region may include a compound represented by Formula 1. For example, in one or more embodiments, the hole injection layer may include a compound represented by Formula 1. For example, in one or more embodiments, the hole transport layer may include a compound represented by Formula 1. For example, in one or more embodiments, the electron blocking layer may include a compound represented by Formula 1. For example, in one or more embodiments, the emission auxiliary layer may include a compound represented by Formula 1.

According to one or more embodiments, the emission layer may include a first host, a second host, and a dopant, wherein the dopant may be a compound including a metal and a ligand including an imidazole moiety.

According to one or more embodiments, the first host may be a hole-transporting host.

According to one or more embodiments, the second host may be an electron-transporting host.

The hole-transporting host may be a compound having strong hole properties. The expression “a compound having strong hole properties” refers to a compound that is easy to accept holes, and such properties may be obtained by including a hole-receiving moiety (also, referred to as a HT moiety).

Such a hole-receiving moiety may include, for example, an electron-rich group or an amine group, and the hole-transporting host may include, for example, a π electron-rich heteroaromatic compound (e.g., a carbazole derivative or an indole derivative) or an aromatic amine compound.

The electron-transporting host may be a compound having strong electron properties. The expression “a compound having strong electron properties” refers to a compound that is easy to accept electrons, and such properties may be obtained by including an electron-receiving moiety (also, referred to as an ET moiety).

Such an electron-receiving moiety may include, for example, an electron-deficient group. For example, the electron-transporting host may include a nitrogen-containing heteroaromatic compound.

When a compound includes only a HT moiety or only an ET moiety, it is clear whether the nature of the compound has HT properties or ET properties.

A compound may include both (e.g., simultaneously) a HT moiety and an ET moiety. In this regard, a simple comparison between the total number of the HT moieties and the total number of the ET moieties in the compound may be a criterion for predicting whether the compound is a hole-transporting (HT) compound or an electron-transporting (ET) compound, but cannot be an absolute criterion. One of the reasons why such a simple comparison cannot be an absolute criterion is that one HT moiety and one ET moiety do not respectively have exactly the same ability to attract holes and electrons.

Therefore, a relatively reliable way to determine whether a compound having a certain structure is HT compound or ET compound is to directly implement the compound in a device.

According to one or more embodiments, a weight ratio of the first host and the second host may be in a range of 9:1 to 1:9. For example, in one or more embodiments, a weight ratio of the first host and the second host may may be in a range of 6:4 to 4:6. When the weight ratio of the first host and the second host is within the ranges above, a balance of injected charges may be appropriate or suitably achieved.

According to one or more embodiments, the metal of the dopant may include a transition metal.

For example, in one or more embodiments, the dopant may include a compound represented by Formula 401:

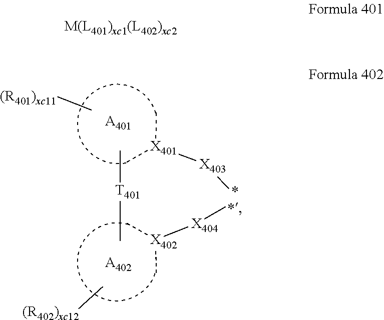

wherein, in Formulae 401 and 402,
M may be titanium (Ti), cobalt (Co), copper (Cu), zinc (Zn), zirconium (Zr), ruthenium (Ru), rhodium (Rh), palladium (Pd), rhenium (Re), platinum (Pt), gold (Au), osmium (Os), iridium (Ir), or rhenium (Re),
L₄₀₁ may be a ligand represented by Formula 402, and xc1 may be 1, 2, or 3, wherein, if (e.g., when) xc1 is 2 or greater, two or more of L₄₀₁ may be substantially identical to or different from each other,
L₄₀₂ may be an organic ligand, and xc2 may be 0, 1, 2, 3, or 4, wherein, if (e.g., when) xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently a C₃-C₆₀ carbocyclic group or a C₁-C₆₀ heterocyclic group,
T₄₀₁ may be a single bond, —O—, —S—, —C(═O)—, —N(Q₄₁₁)-, —C(Q₄₁₁)(Q₄₁₂)-, —C(Q₄₁₁)=C(Q₄₁₂)-, —C(Q₄₁₁)=, or ═C═,
X₄₀₃ and X₄₀₄ may each independently be a chemical bond, O, S, N(Q₄₁₃), B(Q₄₁₃), P(Q₄₁₃), C(Q₄₁₃)(Q₄₁₄), or Si(Q₄₁₃)(Q₄₁₄),
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
optionally, R₄₀₁ and R₄₀₂ may be independently linked to each other to form a ring,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, Q₃₁ to Q₃₃, Q₄₁₁ to Q₄₁₄, and Q₄₀₁ to Q₄₀₃ may each independently be hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof,
xc11 and xc12 may each independently be an integer from 0 to 10, and
* and *′ in Formula 402 are each a binding site to M in Formula 401,
any one selected from among ring A₄₀₁ and ring A₄₀₂ may include an imidazole moiety.

In one or more embodiments, if (e.g., when) xc1 in Formula 401 is 2 or greater, two of ring A₄₀₁ among two or more of L₄₀₁(s) may be optionally linked to each other via T₄₀₂, which is a linking group, and/or two of ring A₄₀₂ among two or more of L₄₀₁ (s) may be optionally linked to each other via T₄₀₃, which is a linking group. T₄₀₂ and T₄₀₃ are each the same as described with respect to T₄₀₁.

According to one or more embodiments, the emission layer may be a phosphorescent emission layer.

In one or more embodiments, the emission layer may be a blue emission layer.

According to one or more embodiments, the interlayer may include a red emission layer, a blue emission layer, and a green emission layer. For example, in one or more embodiments, the interlayer may include the red emission layer, the blue emission layer, and the green emission layer in series; or in parallel on a same plane. For example, in one or more embodiments, the interlayer may include a red emission 1 layer, a blue emission layer, a green emission layer, and a hole injection layer, and the hole injection layer may include a compound represented by the Formula 1.

According to one or more embodiments, the emission layer may include m emission layers,
the interlayer further may include m−1 charge generation layers respectively arranged between two adjacent emission layers among the m emission layers,
m may be an integer greater than or equal to 2, and
any one selected from among m−1 charge generation layers may include the compound represented by Formula 1.

For example, in one or more embodiments, the charge generation layer may include an n-charge generation layer and a p-charge generation layer. For example, the p-charge generation layer may include a compound represented by Formula 1.

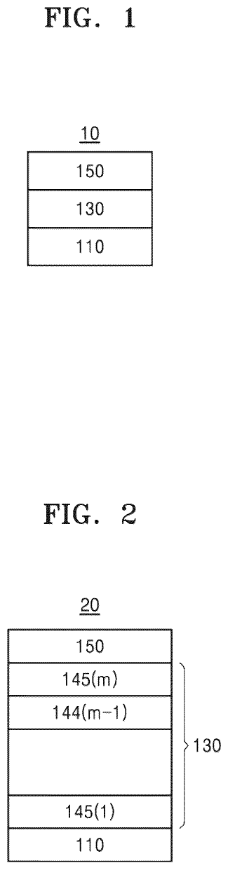

Referring to FIG. 2, which schematically illustrates a light-emitting device 20 according to one or more embodiments of the present disclosure, the light-emitting device 20 may include a first electrode 110, an interlayer 130, and a second electrode 150. The detailed descriptions of the first electrode 110 and the second electrode 150 may refer to the description of FIG. 1 provided herein. The interlayer (130) may include m emission layers 145(1), 145(m−1), 145(m) and (m−1) charge generating layers 144(m−1) respectively arranged between the adjacent emission layers.

For example, if (e.g., when) m is 2, a first electrode, a first emission layer, a first charge generation layer, and a second emission layer may be sequentially arranged. In this case, the first emission layer may be to emit a first color light, the second emission layer may be to emit a second color light, and the maximum emission wavelength (i.e., the wavelength of the maximum emission intensity) of the first color light and the maximum emission wavelength of the second color light may be substantially identical to or different from each other.

As another example, if (e.g., when) m is 3, a first electrode, a first emission layer, a first charge generation layer, a second emission layer, a second charge generation layer, and a third emission layer may be sequentially arranged. In this case, the first emission layer is to emit a first color light, the second emission layer is to emit a second color light, the third emission layer is to emit a third color light, and the maximum emission wavelength of the first color light, the maximum emission wavelength of the second color light, and the maximum emission wavelength of the third color light may be substantially identical to or different from each other.

As another example, if (e.g., when) m is 4, a first electrode, a first emission layer, a first charge generation layer, a second emission layer, a second charge generation layer, a third emission layer, a third charge generation layer, and a fourth emission layer may be sequentially arranged. In this case, the first emission layer is to emit a first color light, the second emission layer is to emit a second color light, the third emission layer is to emit a third color light, the fourth emission layer is to emit a fourth color light, and the maximum emission wavelength of the first color light, the maximum emission wavelength of the second color light, the maximum emission wavelength of the third color light, and the maximum emission wavelength of the fourth color light may be substantially identical to or different from each other.

The same may apply if (e.g., when) m is 5 to 7.

According to one or more embodiments of the present disclosure, an electronic apparatus may include the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode.

According to one or more embodiments, the electronic apparatus may further include a color filter, a color conversion layer, a touch screen layer, a polarizing layer, or any combination thereof. For more details on the electronic apparatus, related descriptions provided herein may be referred to.

The term “interlayer” as used herein refers to a single layer and/or all layers between the first electrode and the second electrode of the light-emitting device.

### Description of FIG. 1

FIG. 1 is a schematic cross-sectional view of a light-emitting device 10 according to one or more embodiments of the present disclosure. The light-emitting device 10 includes a first electrode 110, an interlayer 130, and a second electrode 150.

Hereinafter, a structure of the light-emitting device 10 according to one or more embodiments and a method of manufacturing the light-emitting device 10 will be described in more detail with reference to FIG. 1.

### First Electrode 110

In FIG. 1, in one or more embodiments, a substrate may be additionally provided and arranged under the first electrode 110 and/or on the second electrode 150. As the substrate, a glass substrate or a plastic substrate may be used. In one or more embodiments, the substrate may be a flexible substrate and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a transflective electrode, or a transmissive electrode. In one or more embodiments, if (e.g., when) the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, if (e.g., when) the first electrode 110 is a transflective electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layer structure including (e.g., consisting of) a single layer or a multilayer structure including a plurality of layers. In one or more embodiments, the first electrode 110 may have a three-layer structure of ITO/Ag/ITO.

### Interlayer 130

The interlayer 130 may be on (e.g., arranged on) the first electrode 110. The interlayer 130 may include an emission layer.

The interlayer 130 may further include a hole transport region between (e.g., arranged between) the first electrode 110 and the emission layer, and an electron transport region between (e.g., arranged between) the emission layer and the second electrode 150.

In one or more embodiments, the interlayer 130 may further include, in addition to one or more suitable organic materials, a metal-containing compound such as an organometallic compound, an inorganic material such as quantum dots, and/or the like.

In one or more embodiments, the interlayer 130 may include i) two or more emitting units sequentially stacked between the first electrode 110 and the second electrode 150, and ii) a charge generation layer located between adjacent two of the two or more emitting units. When the interlayer 130 includes the two or more emitting units and the charge generation layer as described herein, the light-emitting device 10 may be a tandem light-emitting device.

### Hole Transport Region in Interlayer 130

The hole transport region may have: i) a single-layer structure including (e.g., consisting of) a single layer including (e.g., consisting of) a single material, ii) a single-layer structure including (e.g., consisting of) a single layer including (e.g., consisting of) a plurality of materials that are different from one another, or iii) a multilayer structure including a plurality of layers including a plurality of materials that are different from one another.

The hole transport region may include a hole injection layer, a hole transport layer, an emission auxiliary layer, an electron blocking layer, or any combination thereof.

In one or more embodiments, the hole transport region may have a multilayer structure including a hole injection layer/hole transport layer structure, a hole injection layer/hole transport layer/emission auxiliary layer structure, a hole injection layer/emission auxiliary layer structure, or a hole injection layer/hole transport layer/electron blocking layer structure, the constituent layers of each structure being stacked sequentially stacked from the first electrode 110 in the stated order.

For example, in one or more embodiments, the hole transport region may have a multilayer structure of a hole injection layer/hole transport layer/emission auxiliary layer, or a hole injection layer/hole transport layer/electron blocking layer, the constituent layers of the multilayer structure being sequentially stacked from the first electrode 110 in the stated order.

In one or more embodiments, the hole transport region may include a compound represented by Formula 201, a compound represented by Formula 202, or any combination thereof:

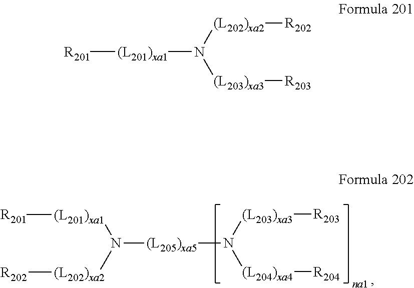

wherein, in Formulae 201 and 202,
L₂₀₁ to L₂₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
L₂₀₅ may be *—O—*′, *—S—*′, *—N(Q₂₀₁)* a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
R₂₀₁ and R₂₀₂ may be optionally linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group (for example, a carbazole group) that is unsubstituted or substituted with at least one R₁₀ₐ (for example, see Compound HT16),
R₂₀₃ and R₂₀₄ may be optionally linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group that is unsubstituted or substituted with at least one R₁₀ₐ, and
na1 may be an integer from 1 to 4.

In one or more embodiments, each of Formulae 201 and 202 may include at least one selected from among groups represented by Formulae CY201 to CY217:

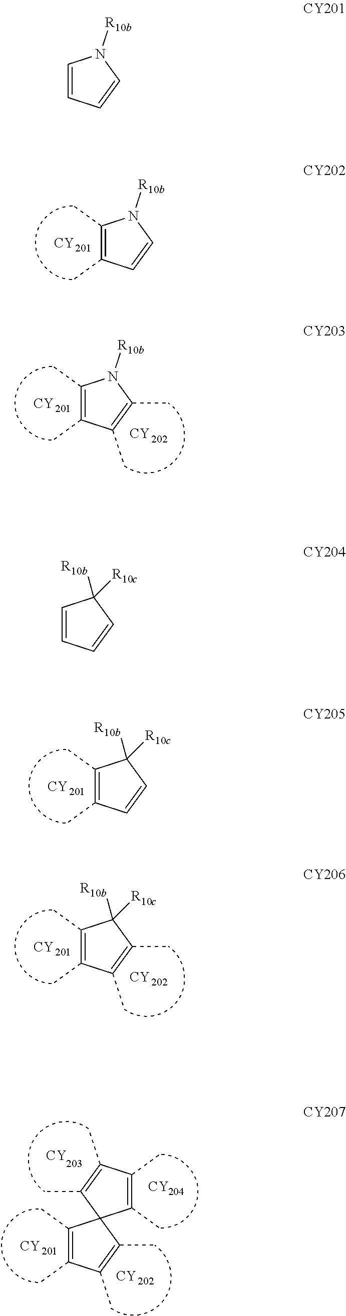

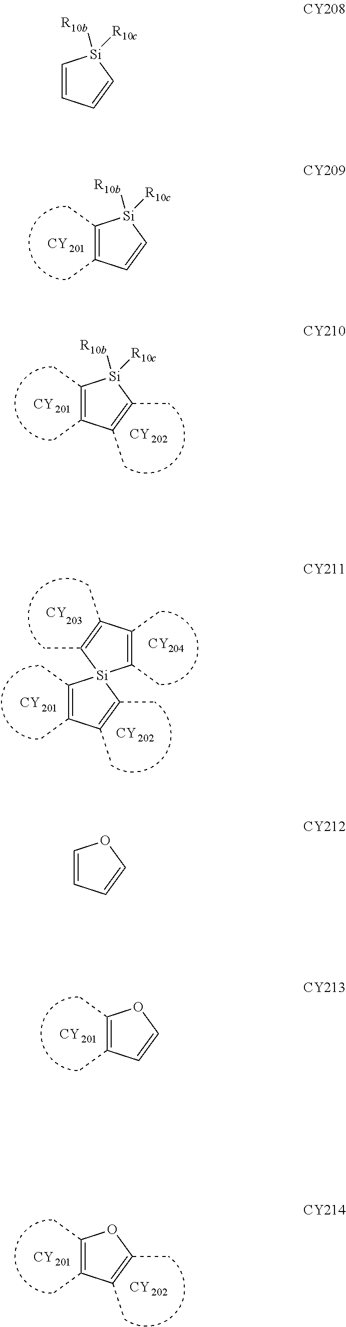

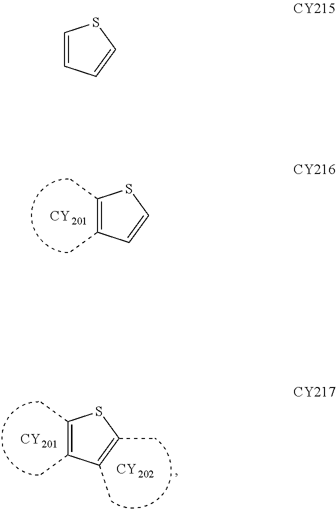

wherein, in Formulae CY201 to CY217, R_{10b}, and R_{10c} are each the same as described with respect to R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one selected from among the groups represented by Formulae CY201 to CY203.

In one or more embodiments, Formula 201 may include at least one selected from among the groups represented by Formulae CY201 to CY203 and at least one selected from among the groups represented by Formulae CY204 to CY217.

In one or more embodiments, in Formula 201, xa1 may be 1, R₂₀₁ may be a group represented by any one selected from among Formulae CY201 to CY203, xa2 may be 0, and R₂₀₂ may be a group represented by any one selected from among Formulae CY204 to CY207.

In one or more embodiments, each of Formulae 201 and 202 may not include (e.g., may exclude) a (e.g., any) group represented by any one selected from among Formulae CY201 to CY203.

In one or more embodiments, each of Formulae 201 and 202 may not include (e.g., may exclude) a (e.g., any) group represented by any one selected from among Formulae CY201 to CY203, and may include at least one selected from among the groups represented by Formulae CY204 to CY217.

In one or more embodiments, each of Formulae 201 and 202 may not include (e.g., may exclude) a (e.g., any) group represented by any one selected from among Formulae CY201 to CY217.

In one or more embodiments, the hole transport region may include one of (e.g., at least one selected from among) Compounds HT1 to HT46, 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), N,N′-di(naphthalen-1-yl)-N,N′-diphenyl-benzidine (NPB(NPD)), p-NPB, N,N′-bis(3-methylphenyl)-N,N′-diphenyl-[1,1′-biphenyl]-4,4′-diamine (TPD), Spiro-TPD, Spiro-NPB, methylated NPB, 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/OSA), polyaniline/poly(4-styrenesulfonate) (PANI/PSS), or any combination thereof:

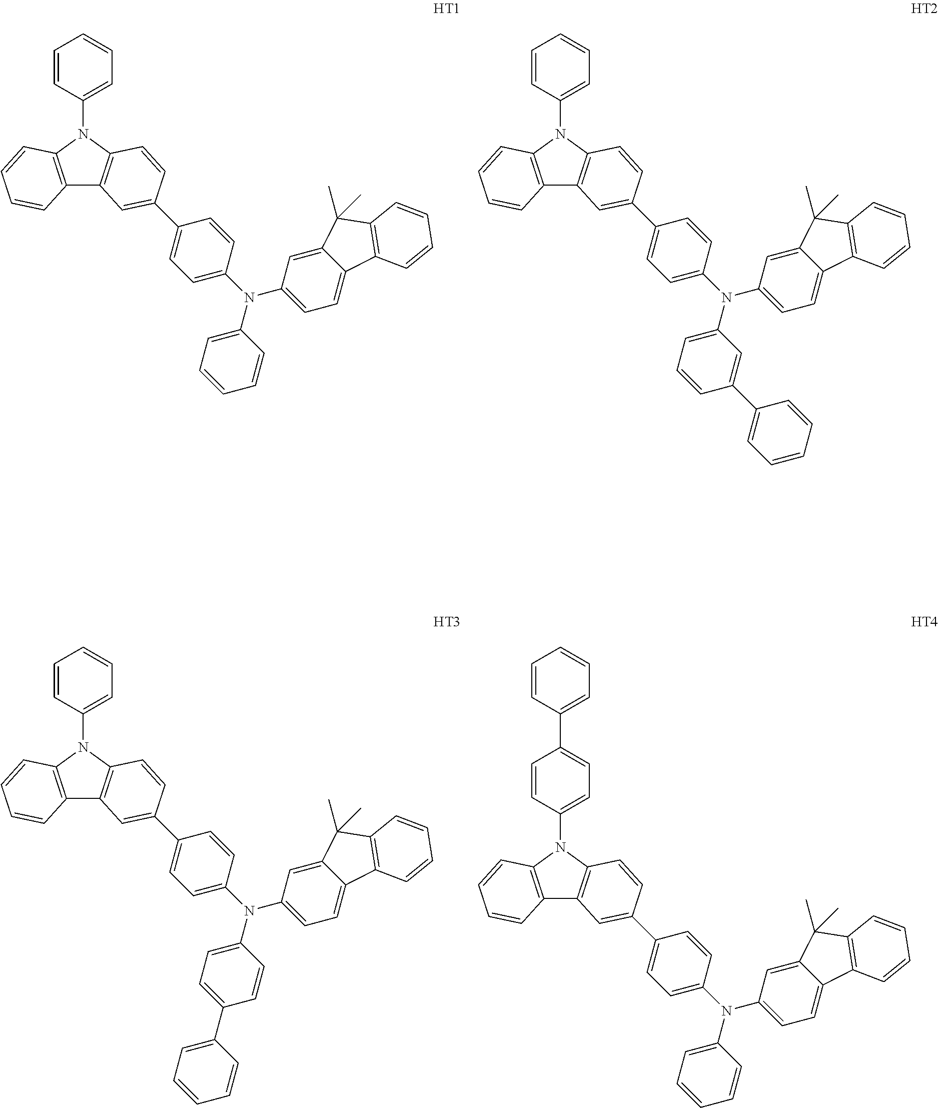 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

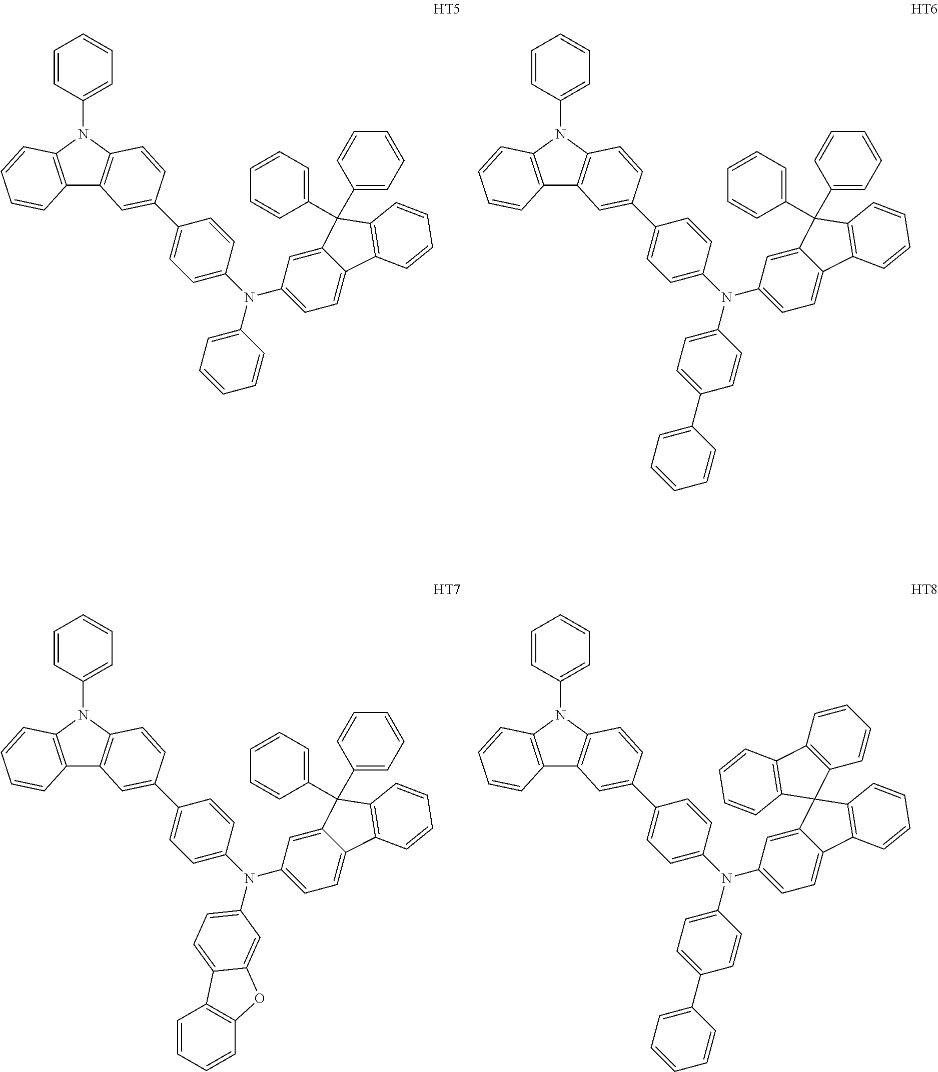 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

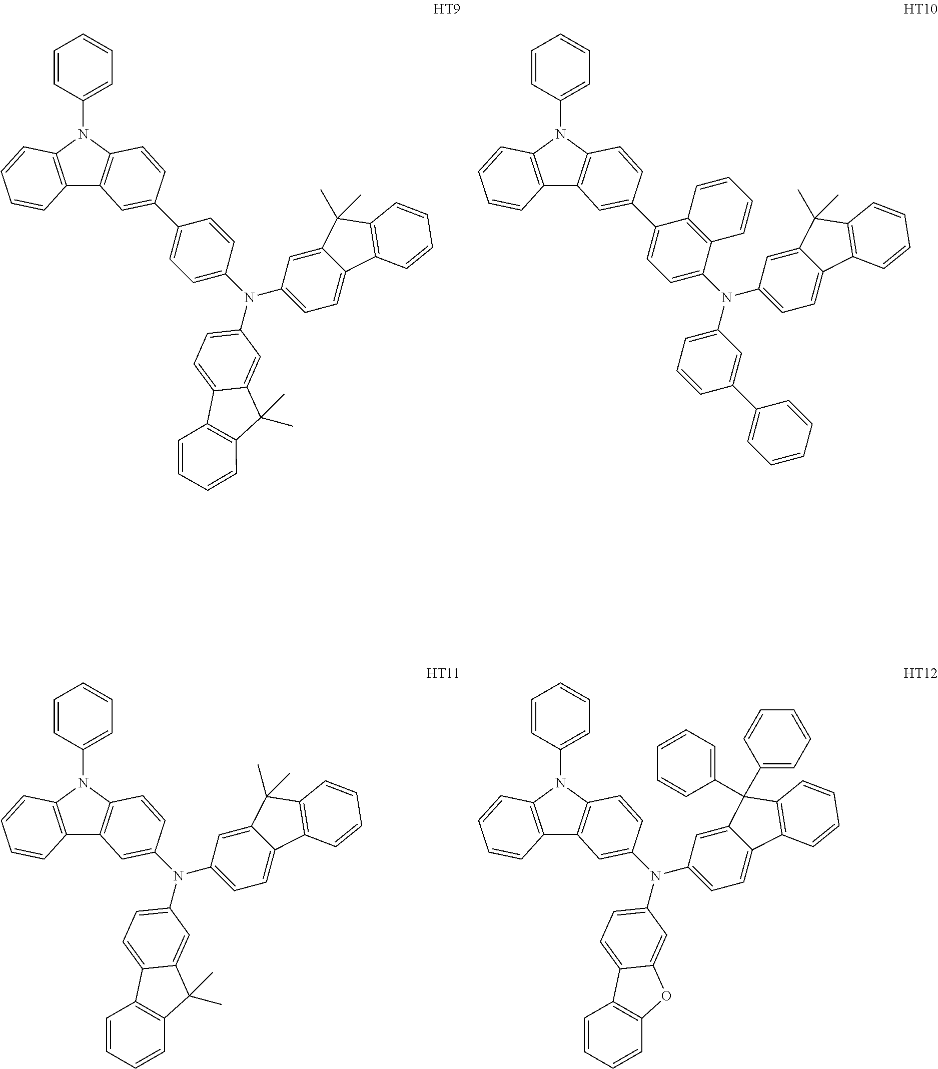 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

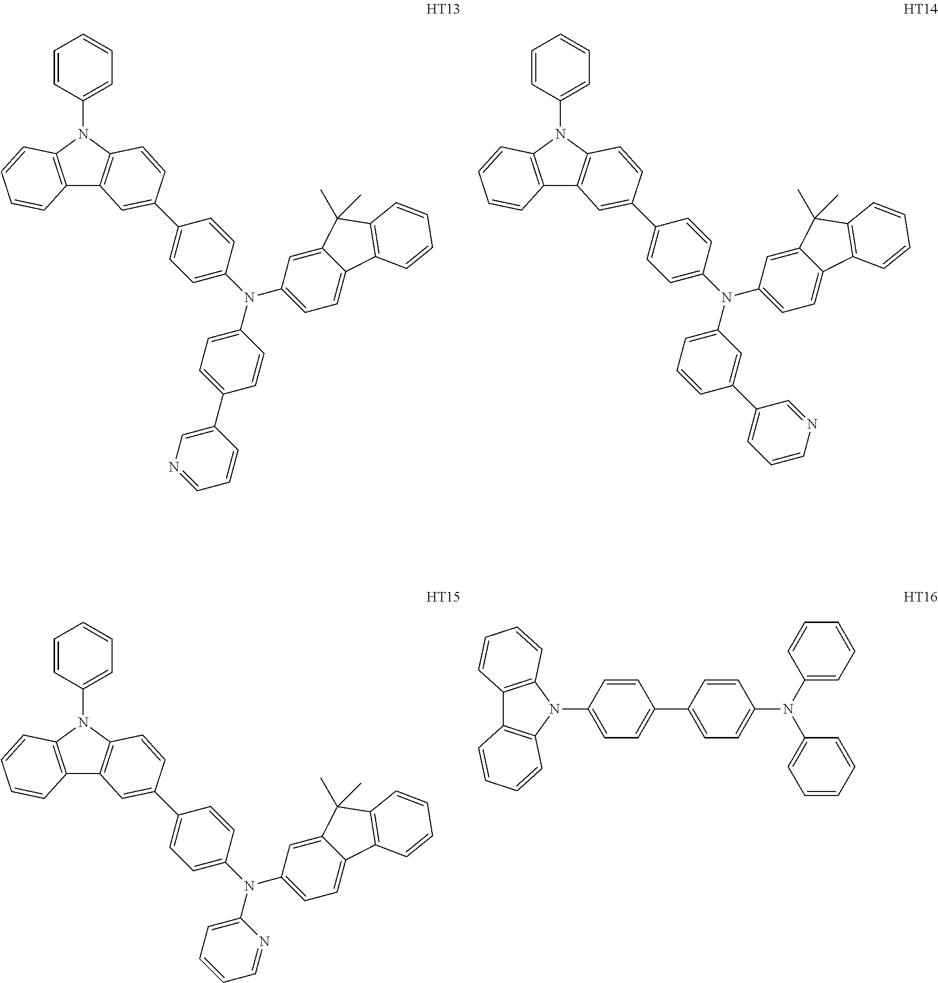 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

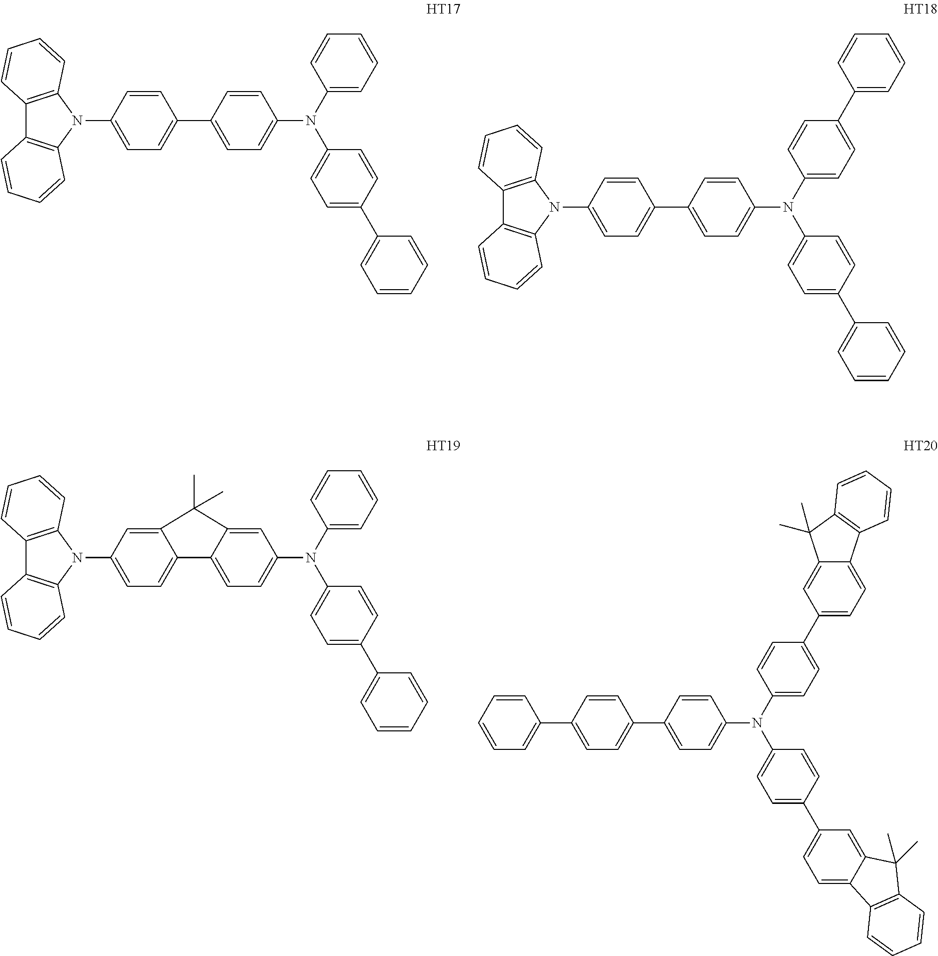 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

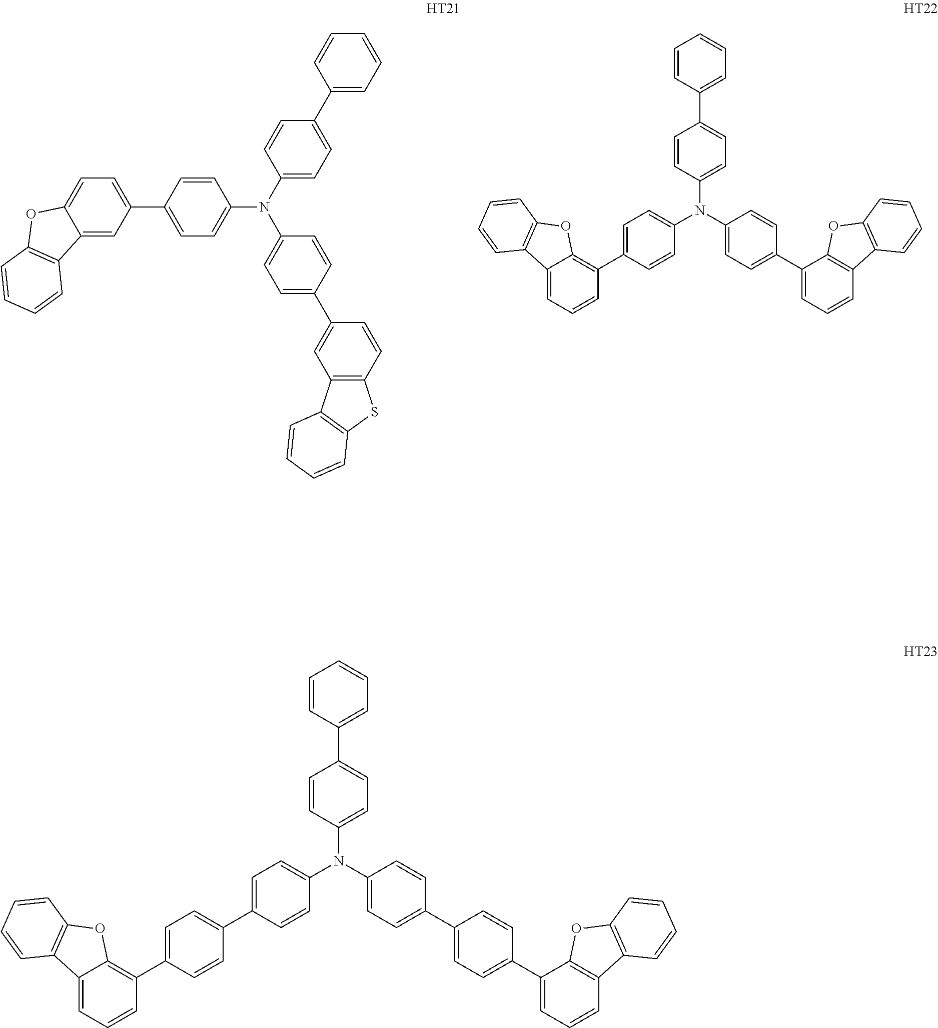 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

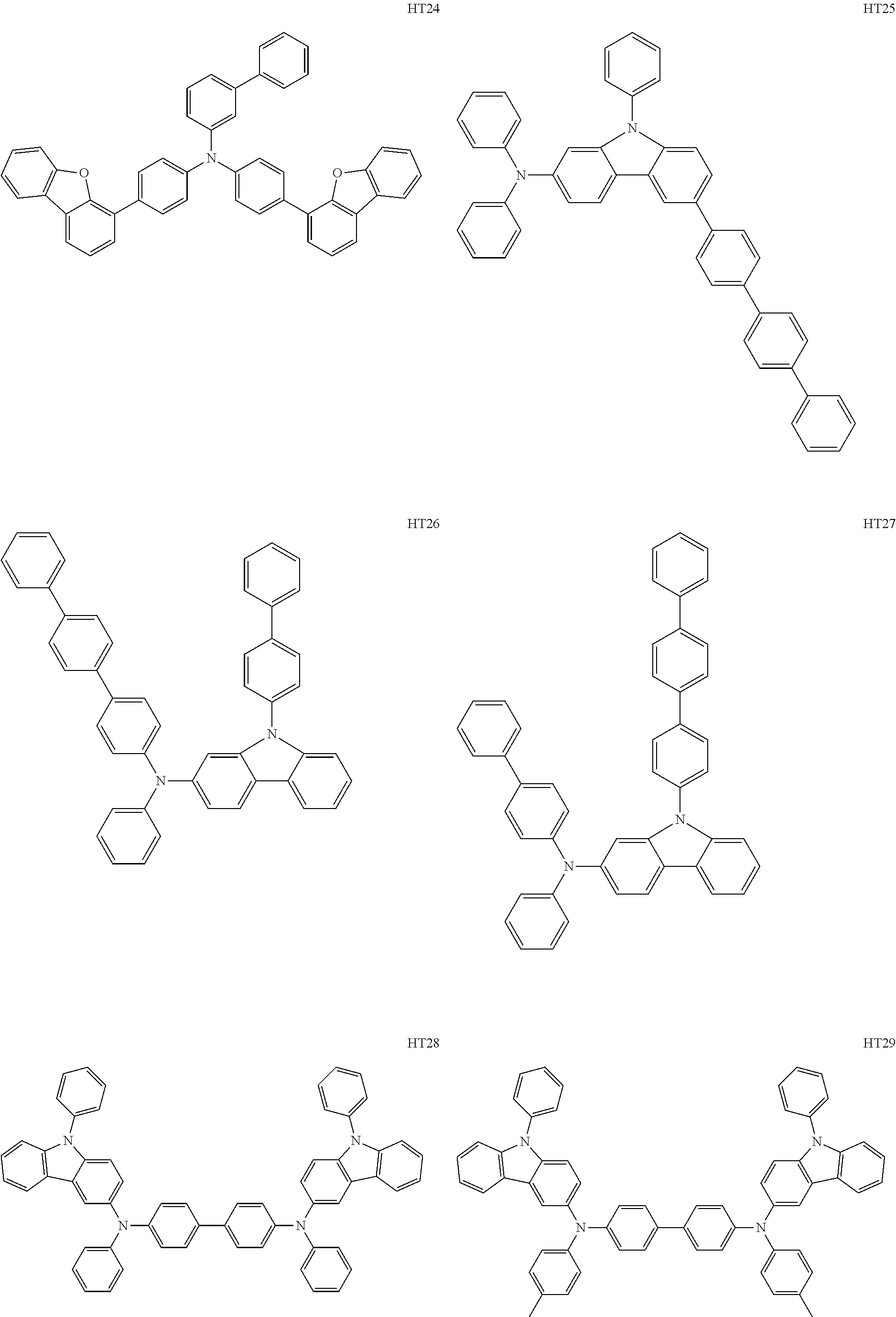

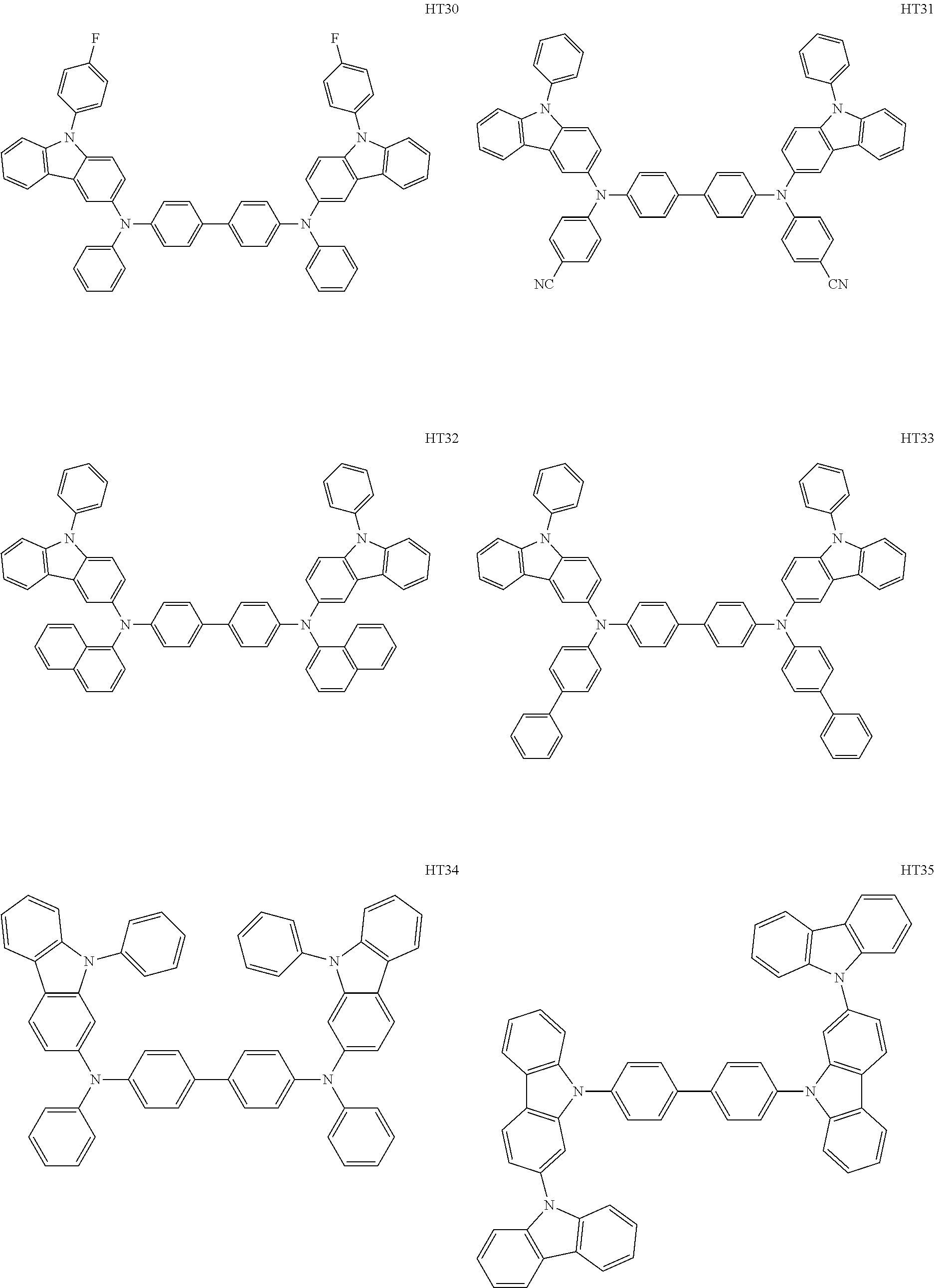

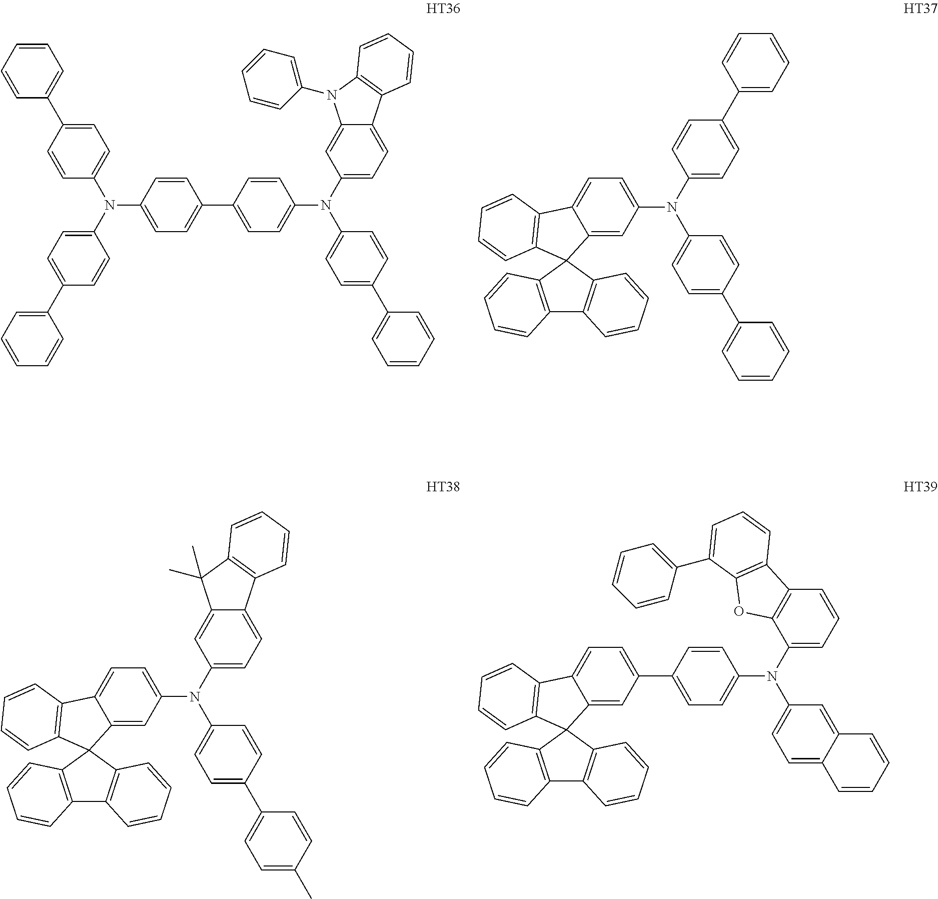 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

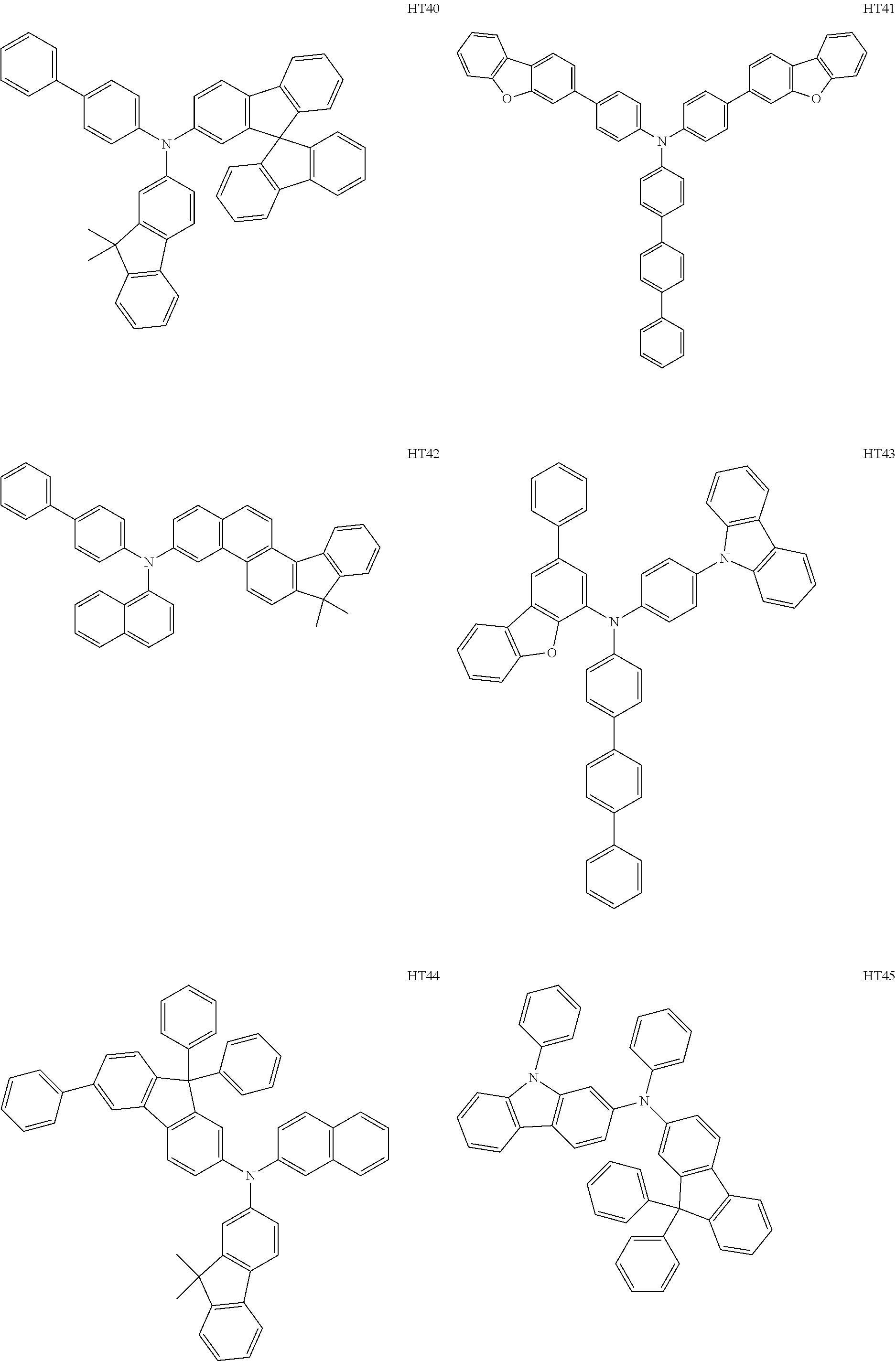

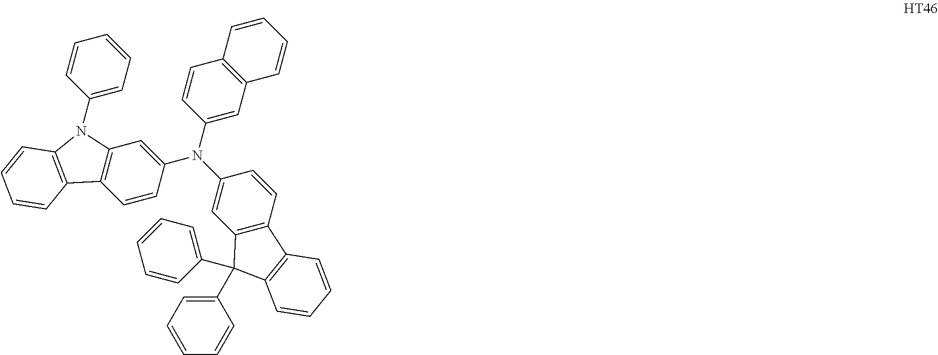 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

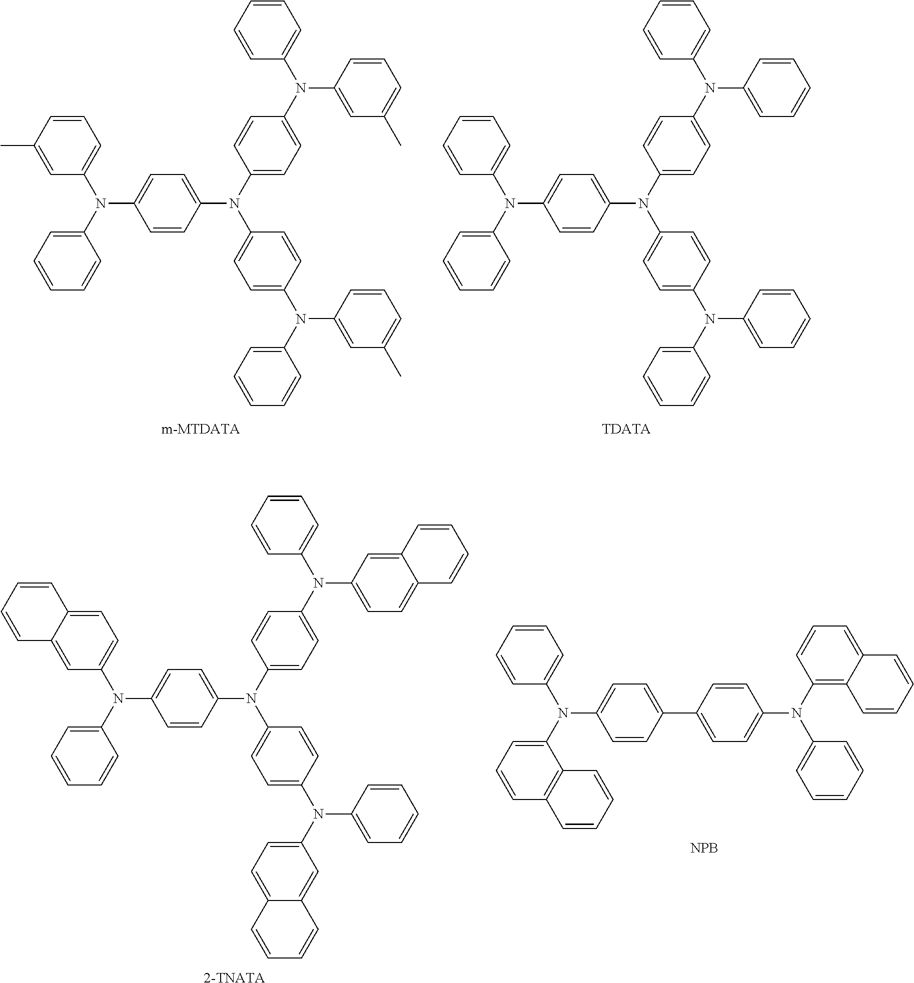

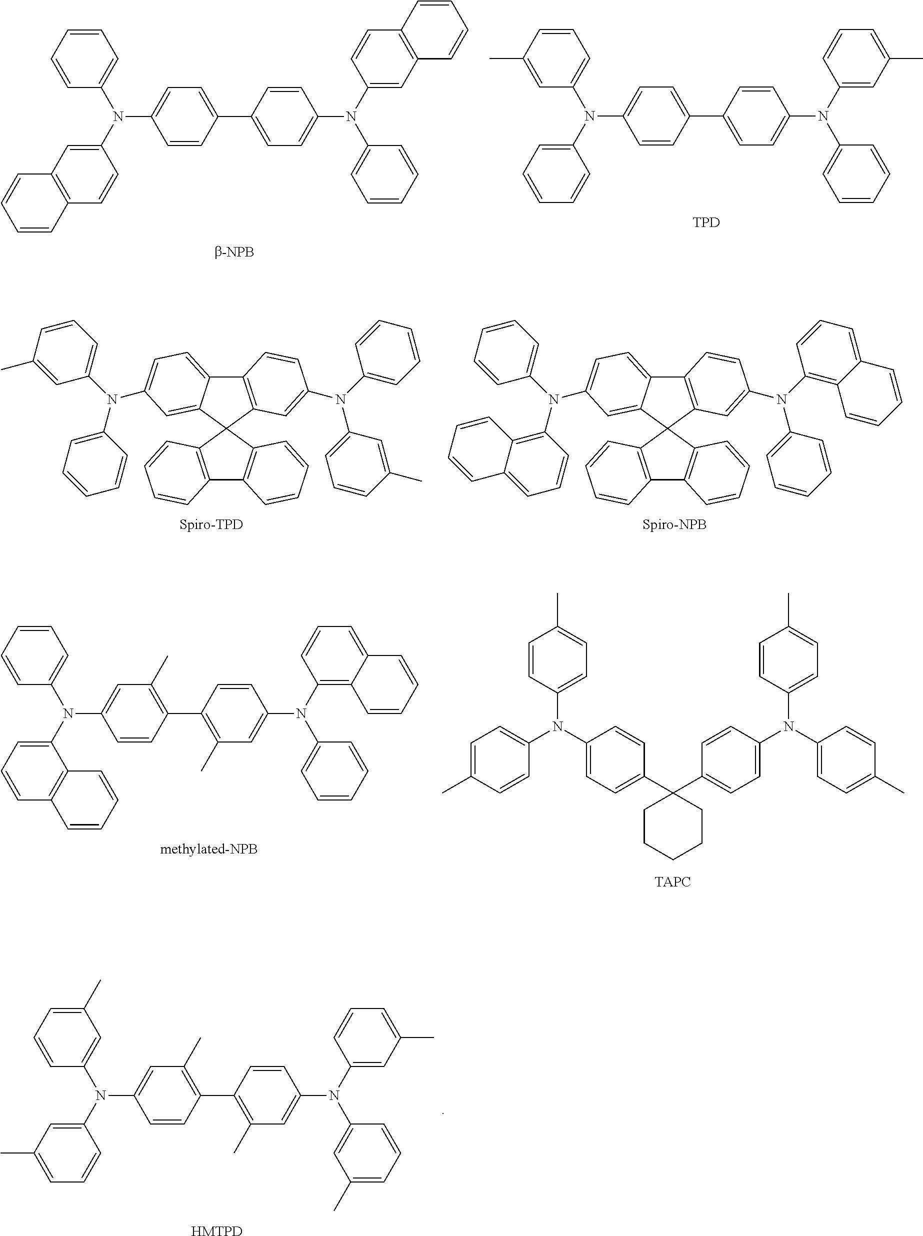

A thickness of the hole transport region may be in the range of about 50 Angstroms (Å) to about 10,000 Å, for example, about 100 Å to about 4,000 Å. A thickness of the hole injection layer may be in the range of about 10 Å to about 1000 Å, for example from about 20 Å to about 500 Å. When the thickness of the hole injection layer is within the ranges described above, satisfactory electron injection characteristics may be obtained without a substantial increase in driving voltage.

According to one or more embodiments, the hole injection layer may include the compound of Formula 1 as a p-dopant.

The emission auxiliary layer may increase light-emission efficiency by compensating for an optical resonance distance according to the wavelength of light emitted from the emission layer, and the electron-blocking layer may block the leakage of electrons from the emission layer to the hole transport region. Materials that may be included in the hole transport region may be included in the emission auxiliary layer and the electron blocking layer.

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers selected from among a red emission layer, a green emission layer, and a blue emission layer, in which the two or more layers contact each other or are separated from each other, to emit white light (e.g., combined white light). In one or more embodiments, the emission layer may include two or more materials selected from among a red light-emitting material, a green light-emitting material, and a blue light-emitting material, in which the two or more materials are mixed with each other in a single layer, to emit white light (e.g., combined white light).

The emission layer may include a host and a dopant. The host may include the first host and the second host. The dopant may include the dopant described above. For example, the dopant may include a phosphorescent dopant.

An amount of the dopant in the emission layer may be in a range of about 0.01 parts by weight to about 15 parts by weight based on 100 parts by weight of the total host.

In one or more embodiments, the emission layer may include a delayed fluorescence material. The delayed fluorescence material may function as a host or a dopant in the emission layer.

A thickness of the emission layer may be in a range of about 100 Å to about 1,000 Å, for example, about 200 Å to about 600 Å. For example, if (e.g., when) the interlayer includes m emission layers, where m is an integer of 2 or greater, the thickness of each emission layer may be in the range of 50 Å to 500 Å. When the thickness of the emission layer is within the range described above, excellent or suitable luminescence characteristics may be obtained without a substantial increase in driving voltage.

### Host in Emission Layer

The host may include the first host and the second host.

In one or more embodiments, the first host and second host may independently include a compound represented by Formula 301.

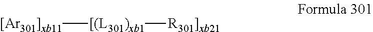

In Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to 0303 may each be the same as described with respect to Q₁.

In one or more embodiments, if (e.g., when) xb11 in Formula 301 is 2 or greater, two or more of Ar₃₀₁(s) may be linked to each other via a single bond.

In one or more embodiments, the first host and the second host may each independently include a compound represented by Formula 301-1, a compound represented by Formula 301-2, or any combination thereof:

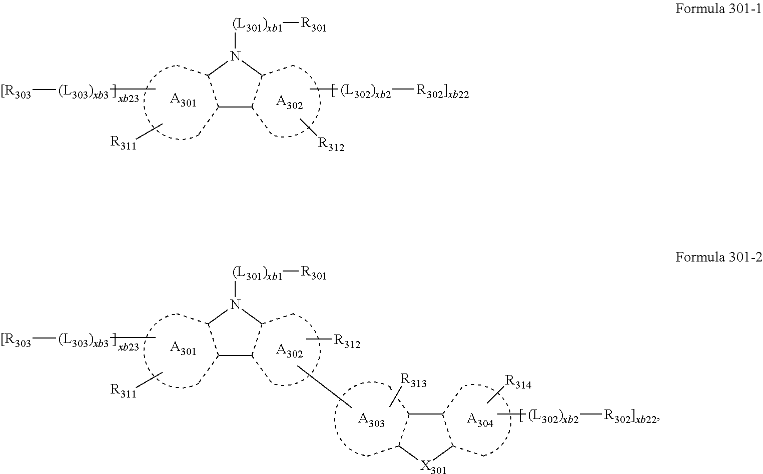

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one 11a,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as described herein,
L₃₀₂ to L₃₀₄ may each independently be the same as described with respect to L₃₀₁,
xb2 to xb4 may each independently be the same as described with respect to xb1, and
R₃₀₂ to R₃₀₅ and R₃₁₁ to R₃₁₄ may each be the same as described herein with respect to R₃₀₁.

In one or more embodiments, the first host and the second host may each independently include an alkaline earth metal complex, a transition metal complex, or any combination thereof. In one or more embodiments, the host may include a Be complex (for example, Compound H55), an Mg complex, a Zn complex, or any combination thereof.

In one or more embodiments, the first host and the second host may each independently include one (e.g., at least one) selected from among the following compounds H1 to H130, ADN (9,10-Di(2-naphthyl)anthracene), MADN (2-Methyl-9,10-bis(naphthalen-2-yl)anthracene), TBADN (9,10-di-(2-naphthyl)-2-t-butyl-anthracene), CBP (4,4′-bis(N-carbazolyl)-1,1′-biphenyl), mCP (1,3-di(carbazol-9-yl)benzene), TCP (1,3,5-tri(carbazol-9-yl)benzene), or any combination thereof:

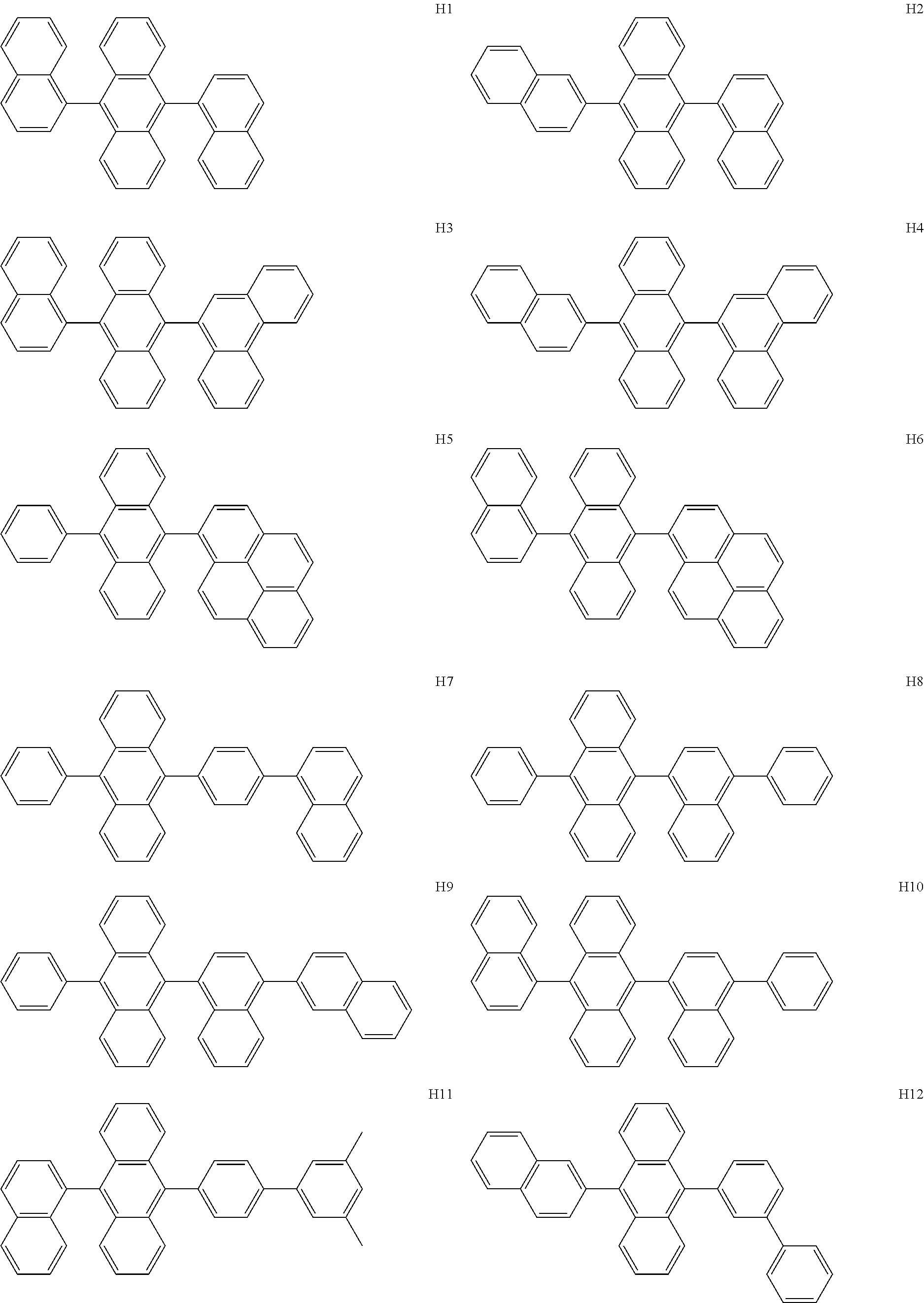

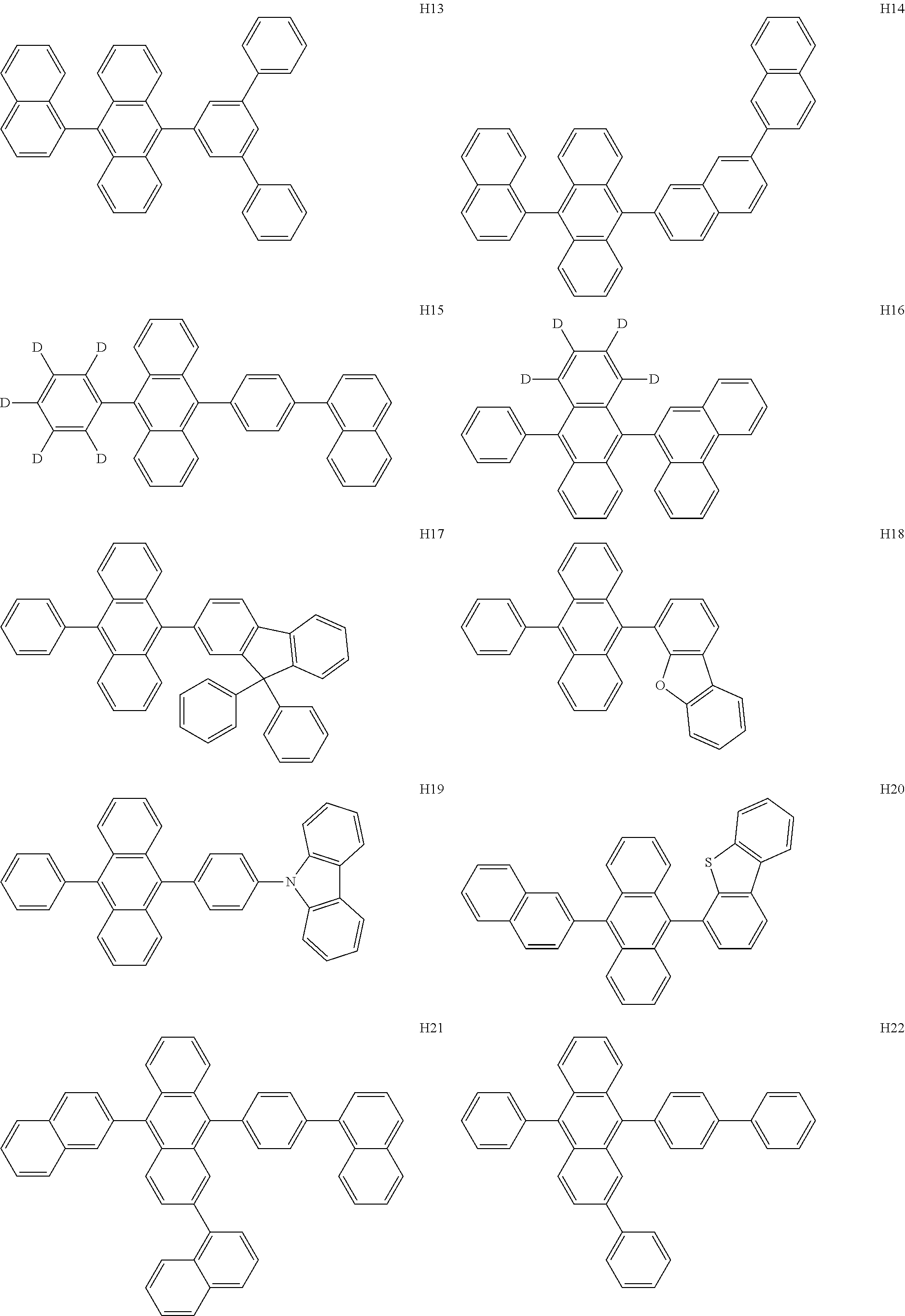

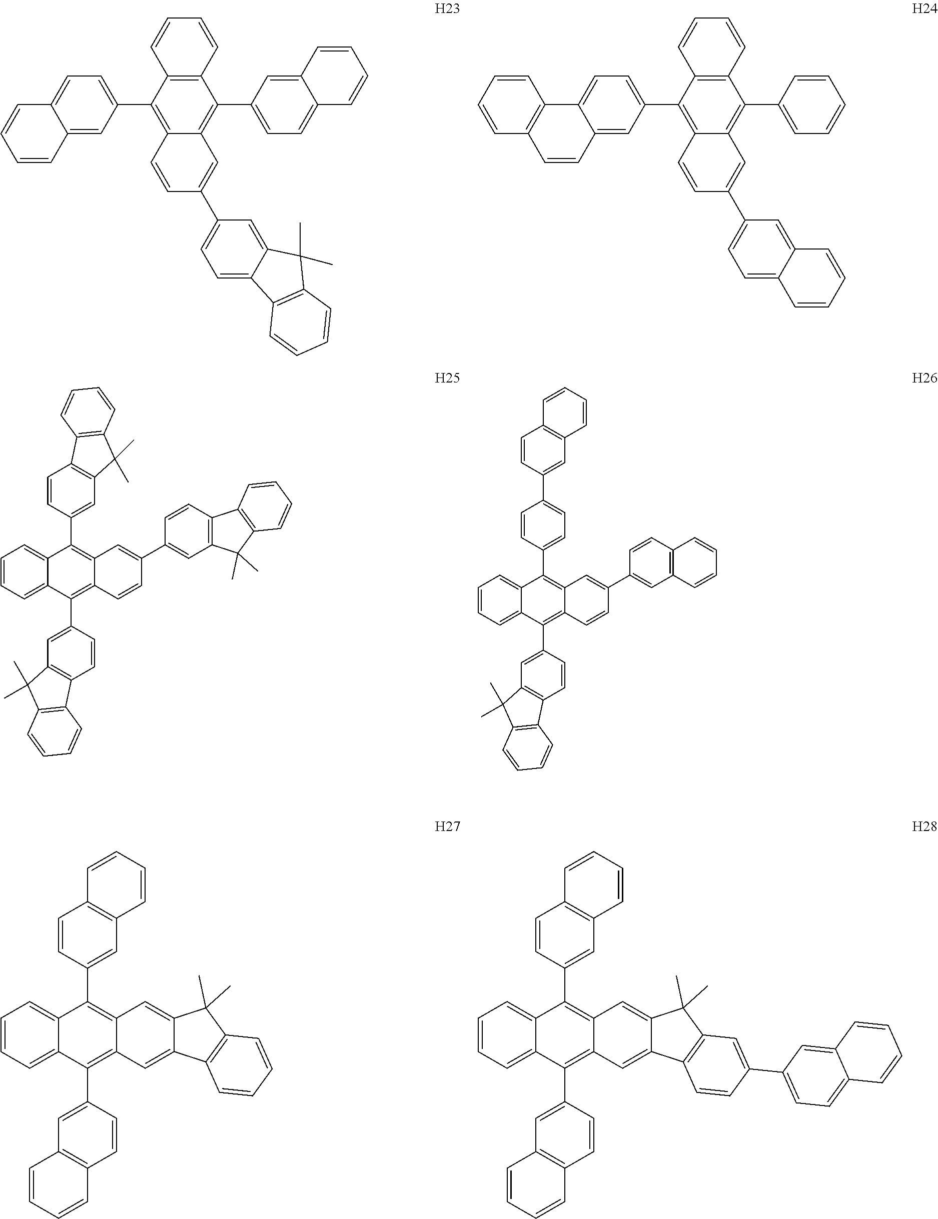

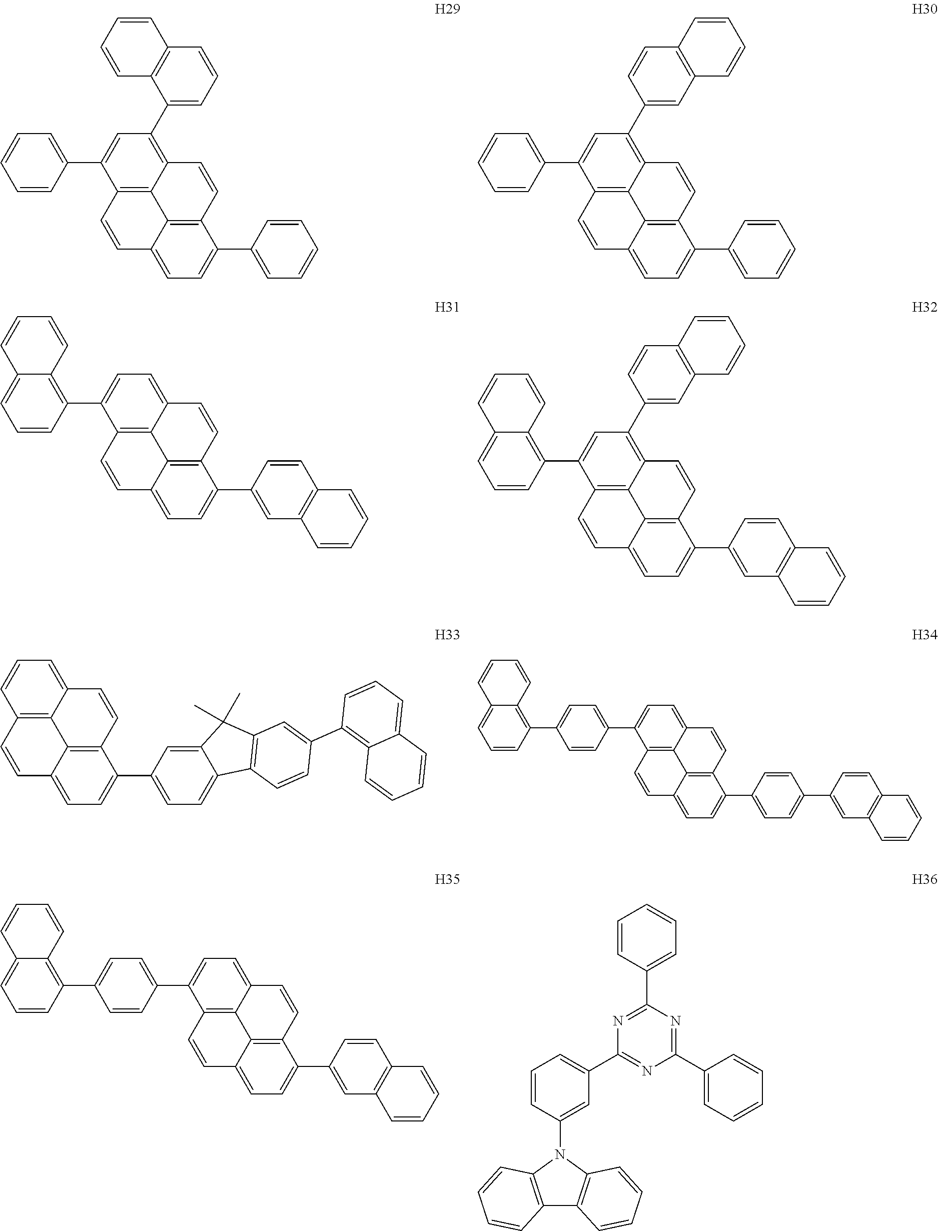

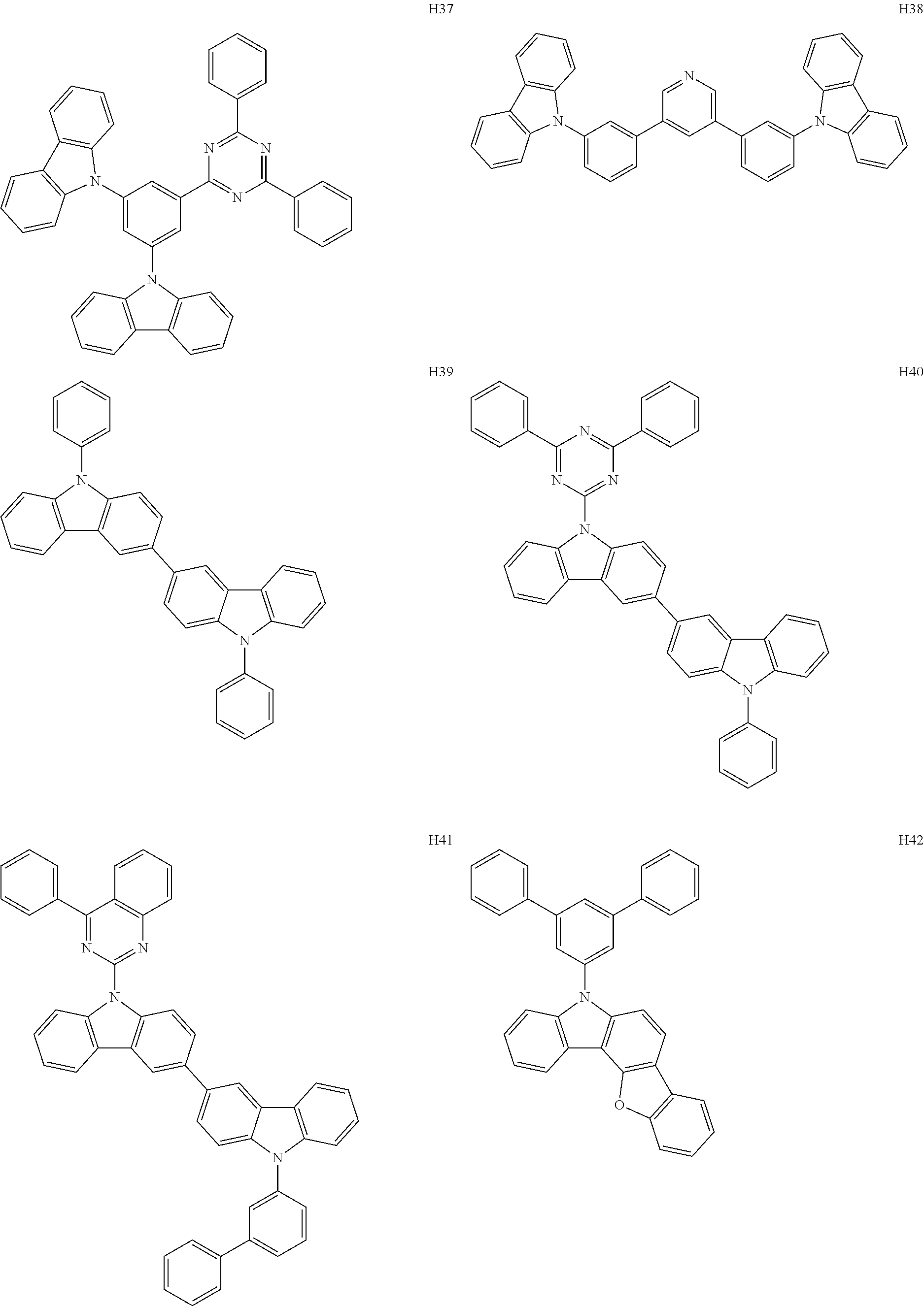

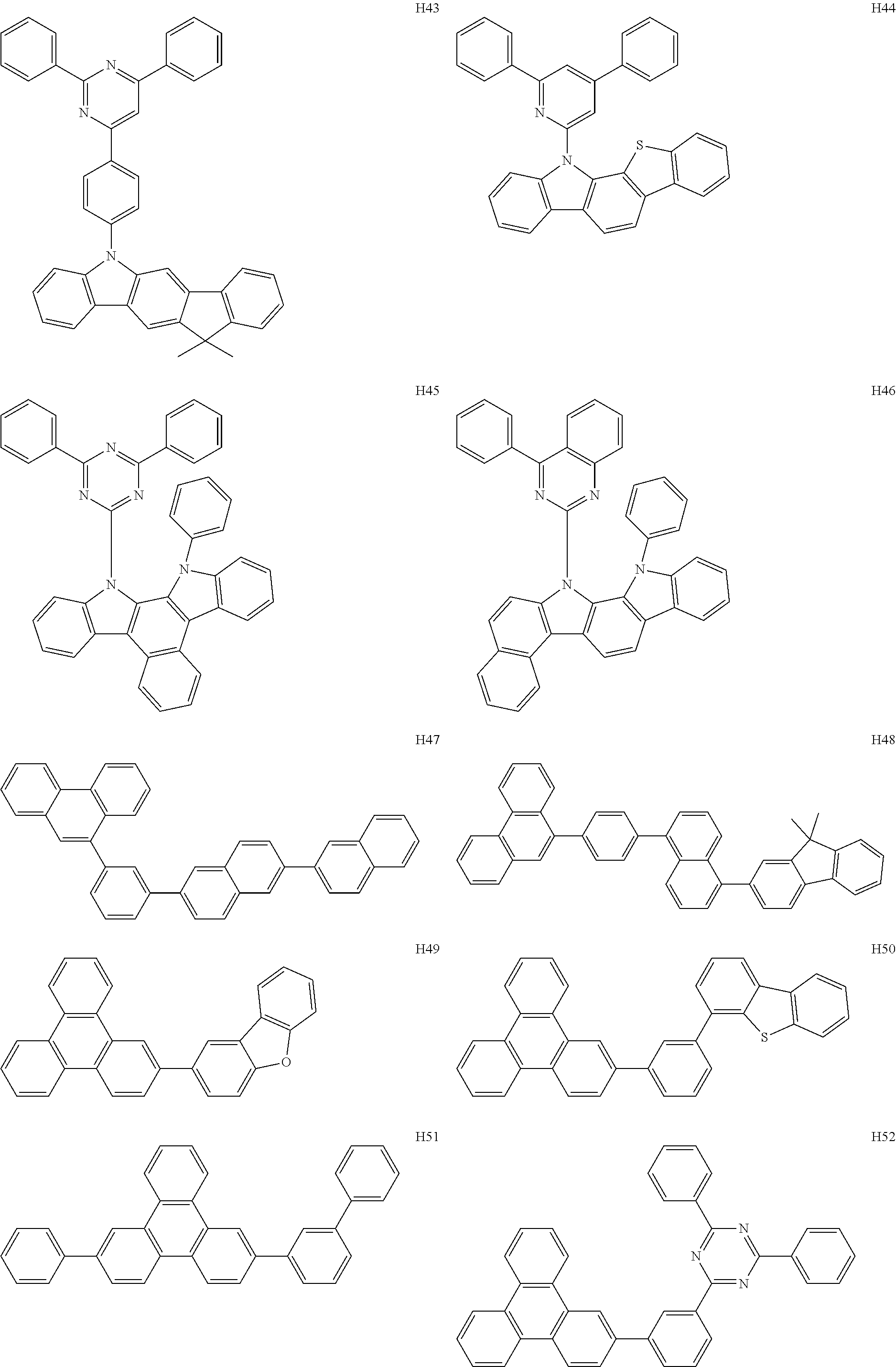

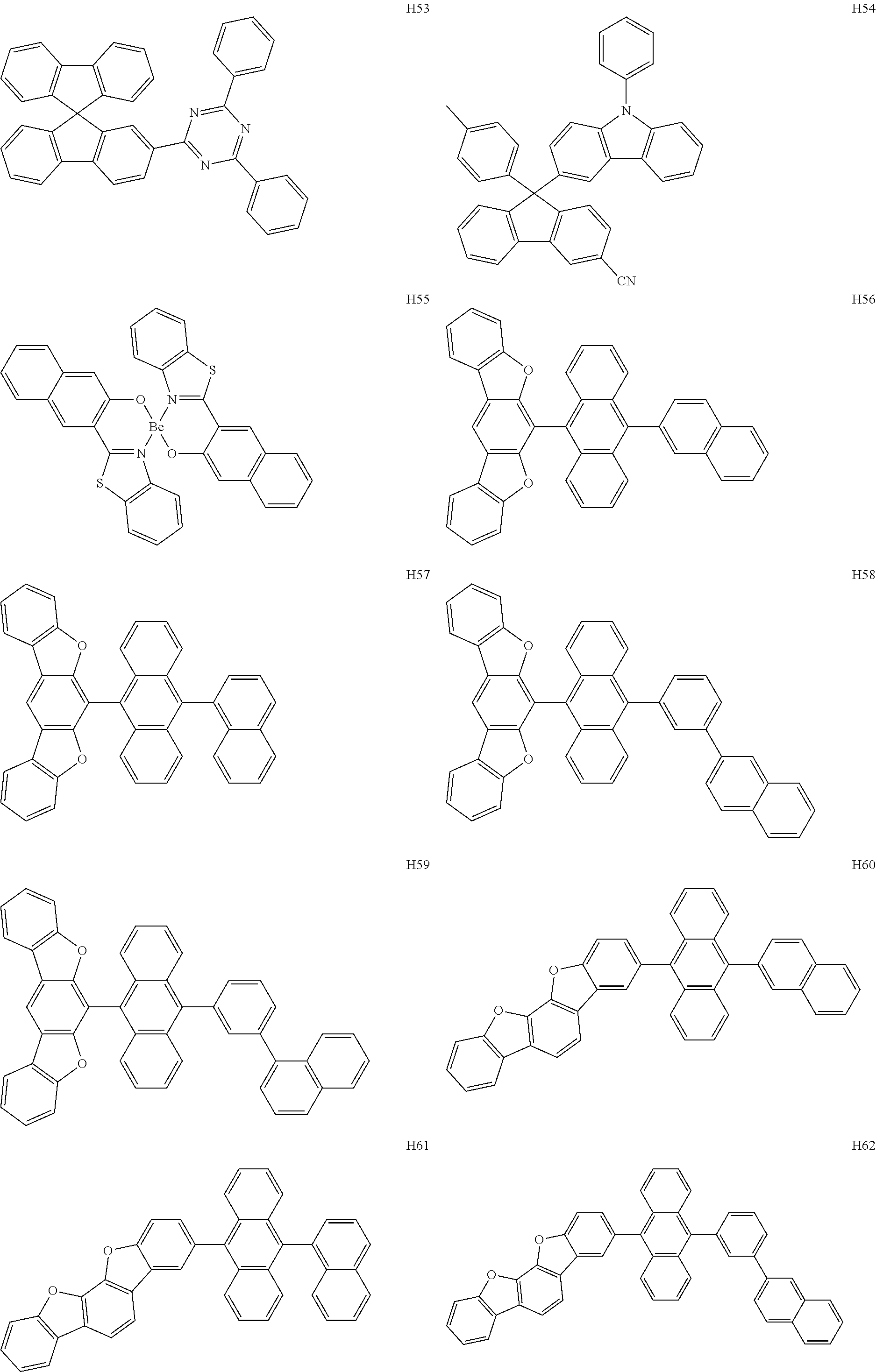

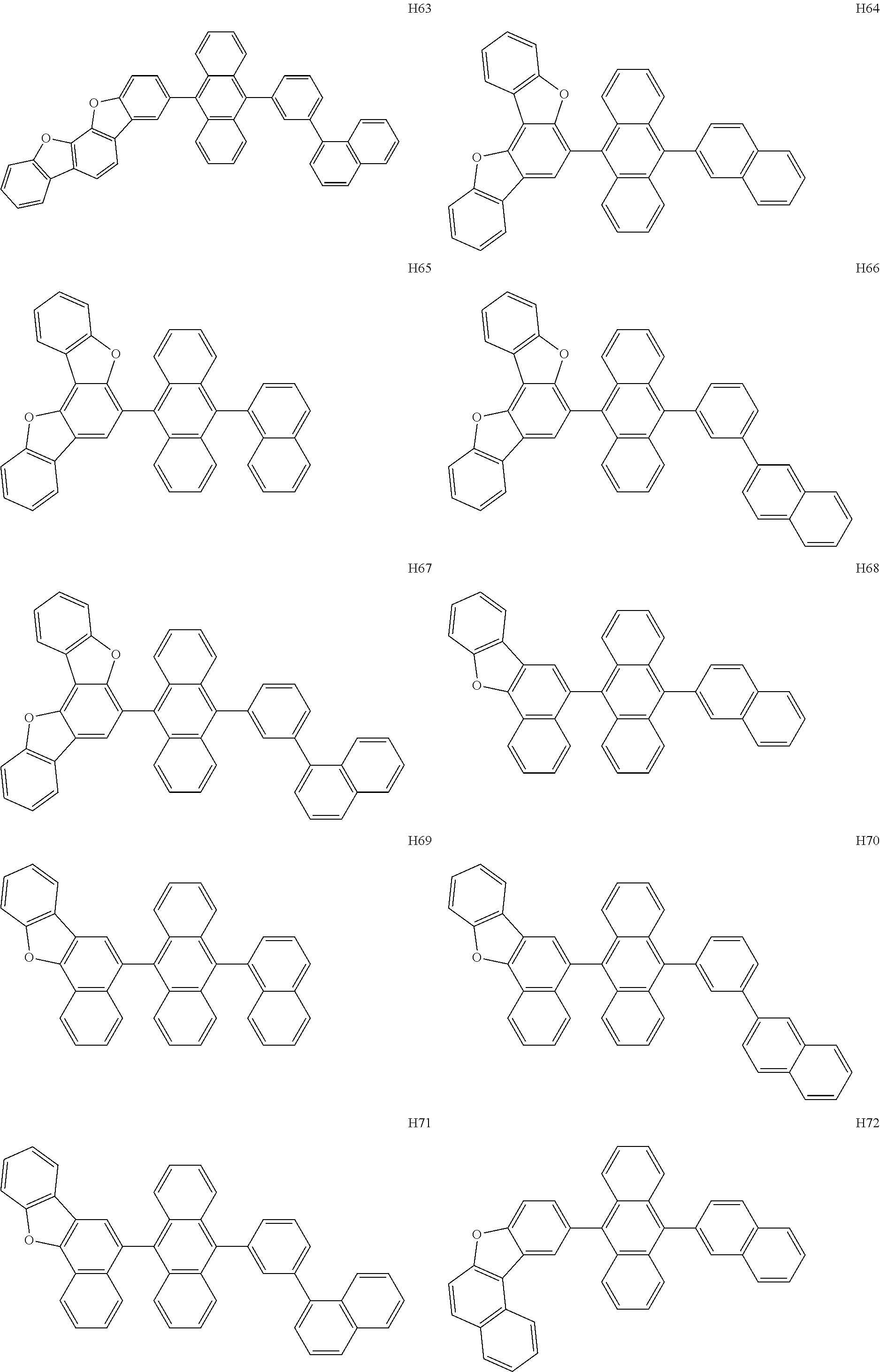

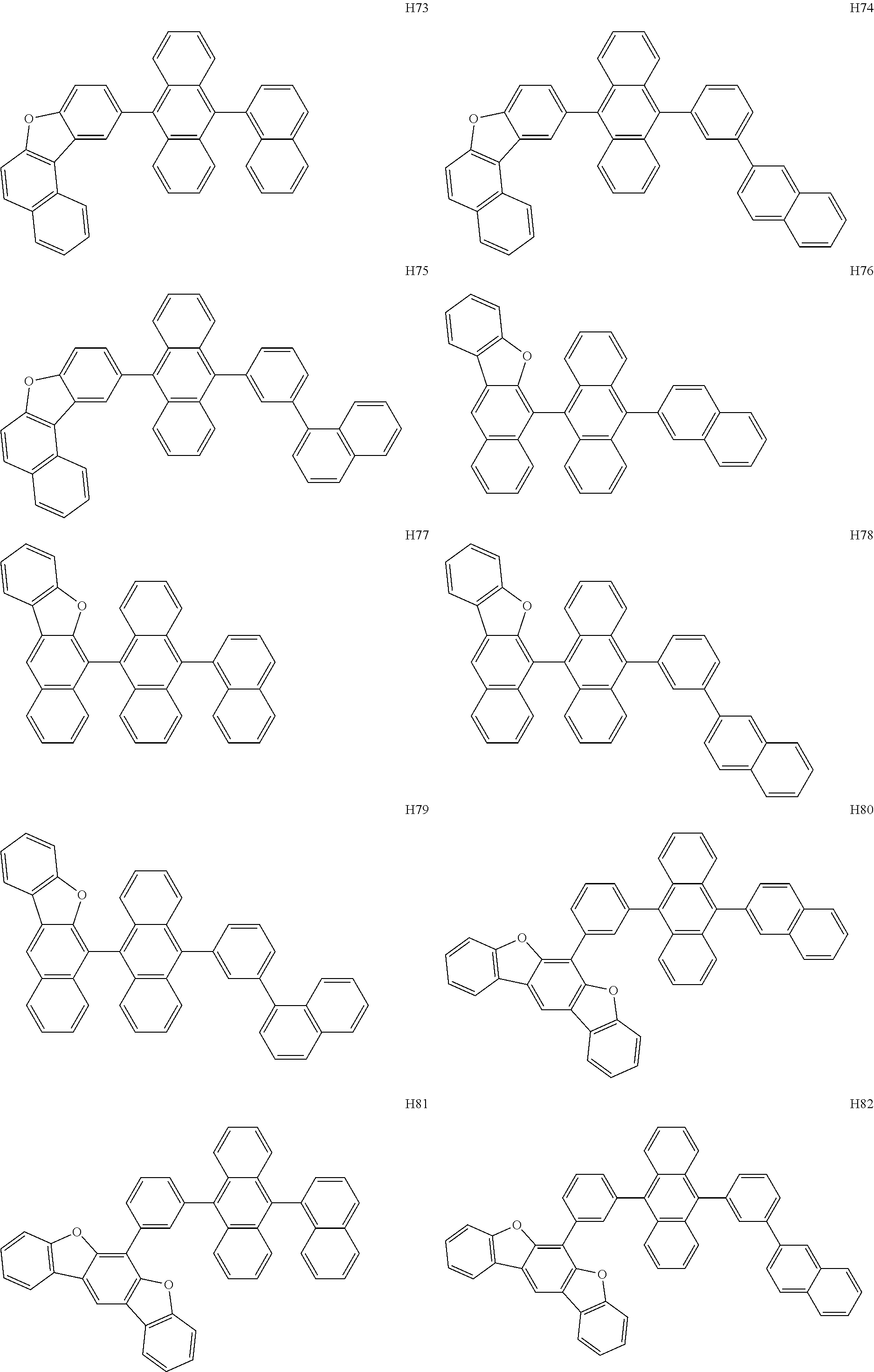

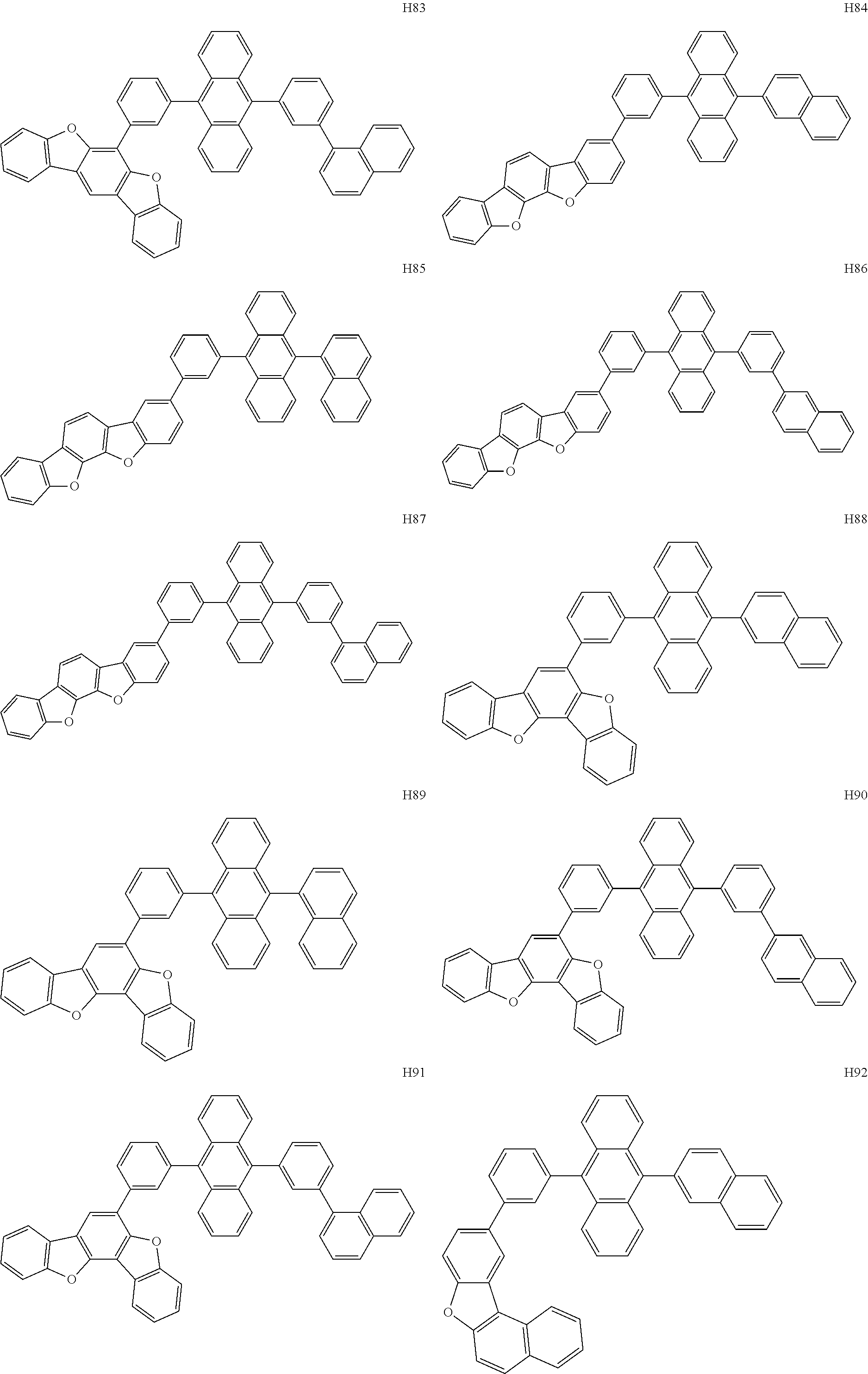

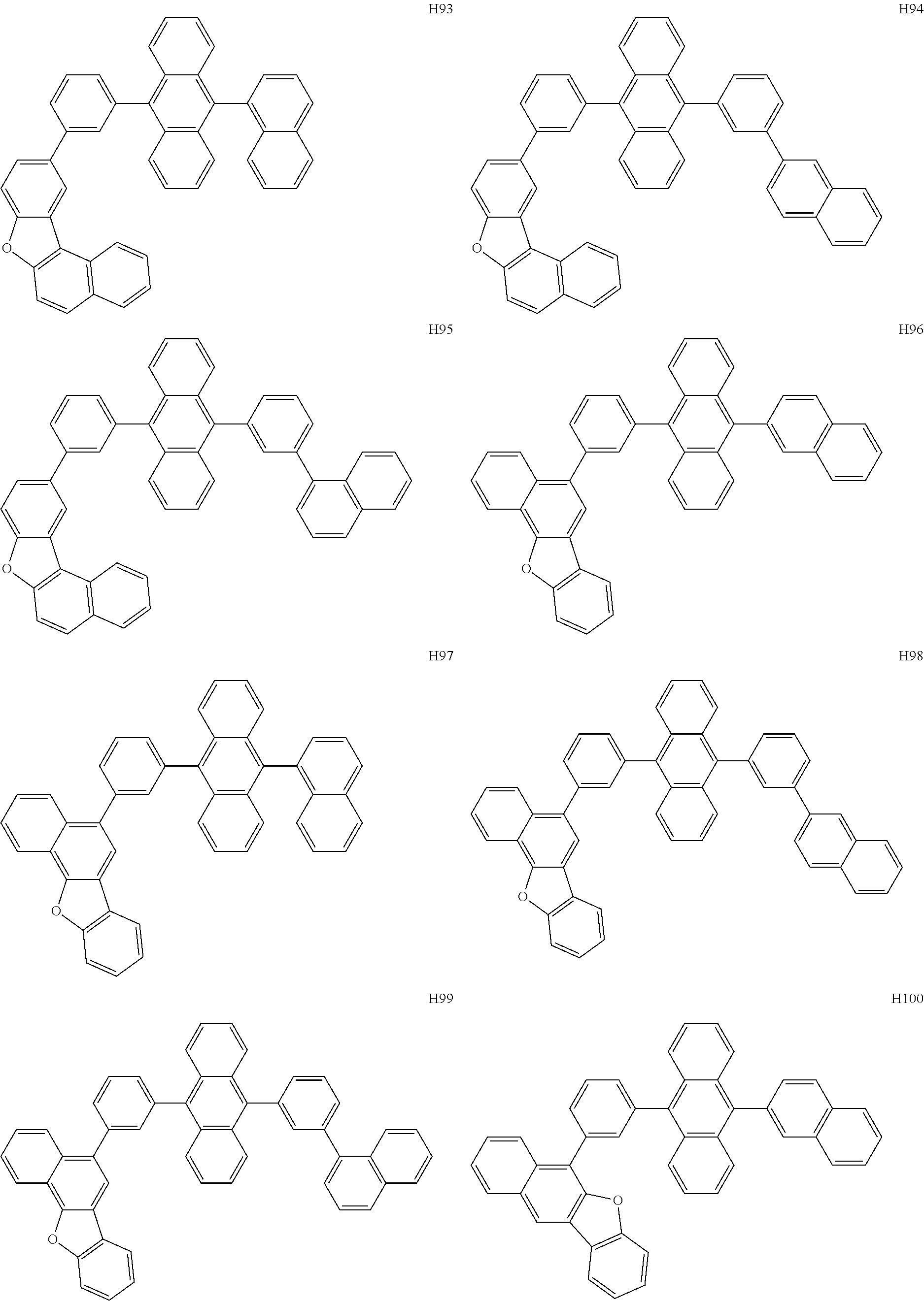

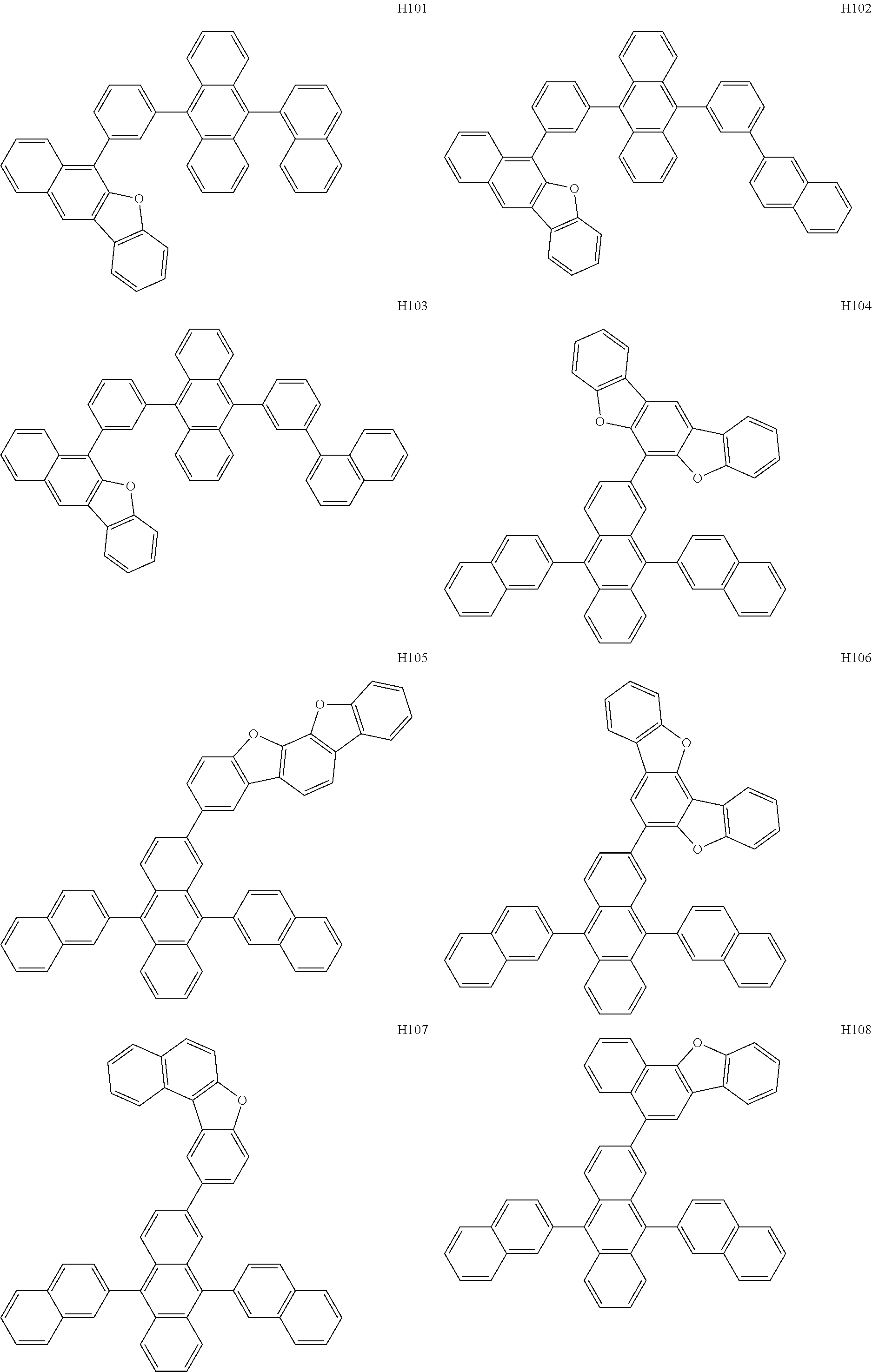

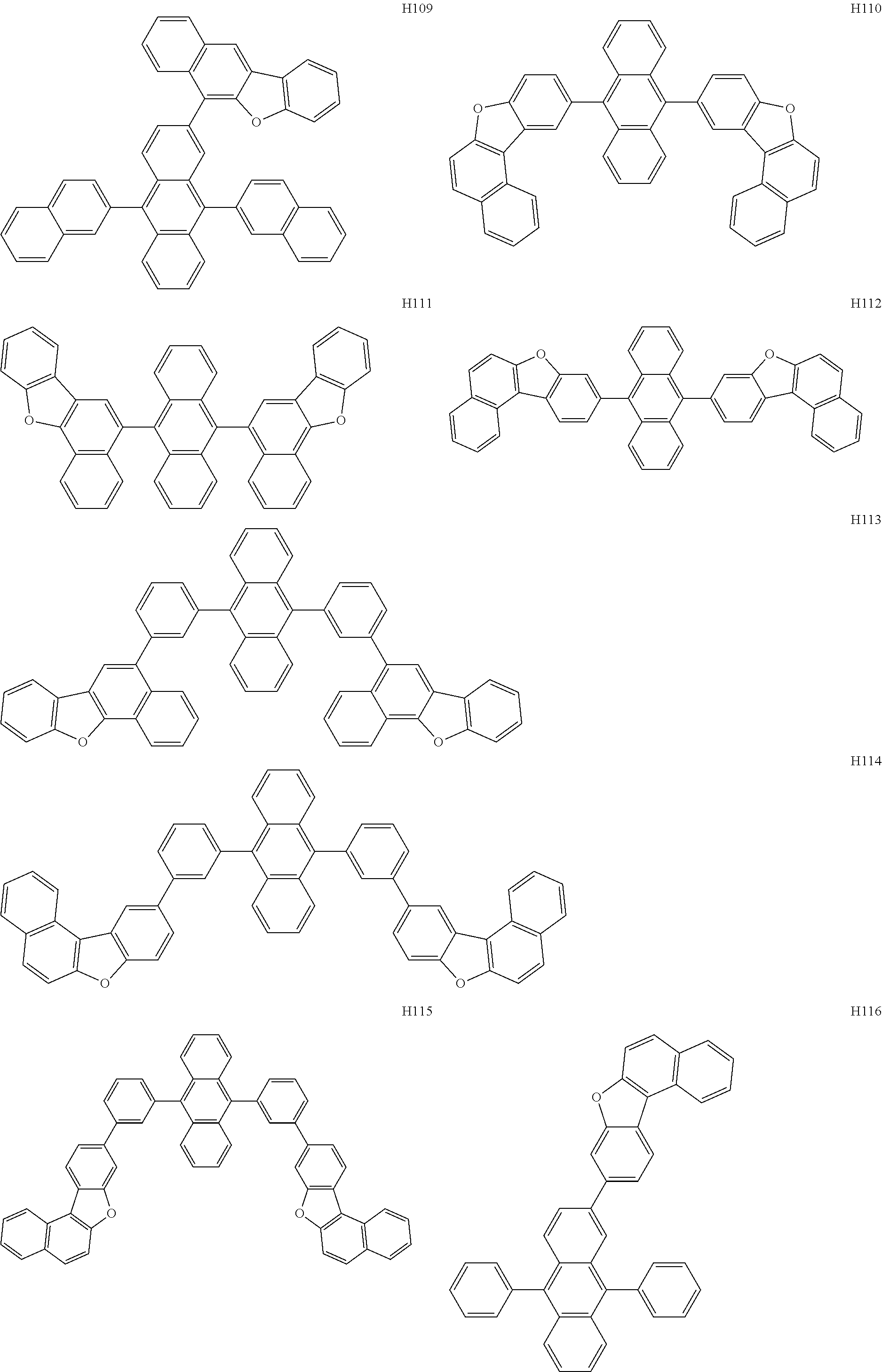

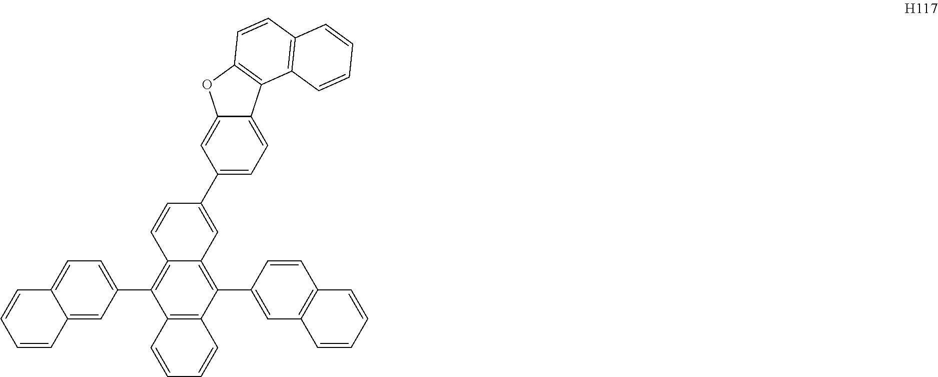 [SMILES: c1ccc2cc(-c3c4ccccc4c(-c4ccc5ccccc5c4)c4cc(-c5ccc6c(c5)oc5ccc7ccccc7c56)ccc34)ccc2c1]

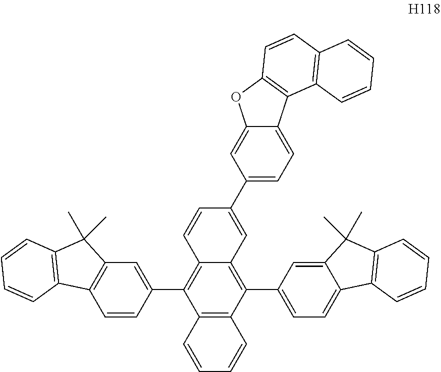 [SMILES: CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

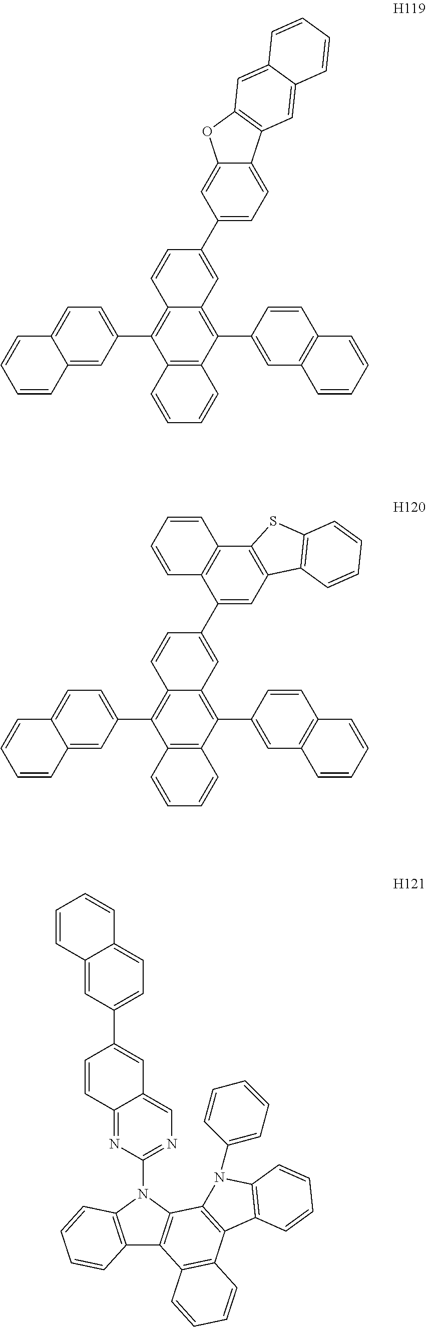 [SMILES: c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1]

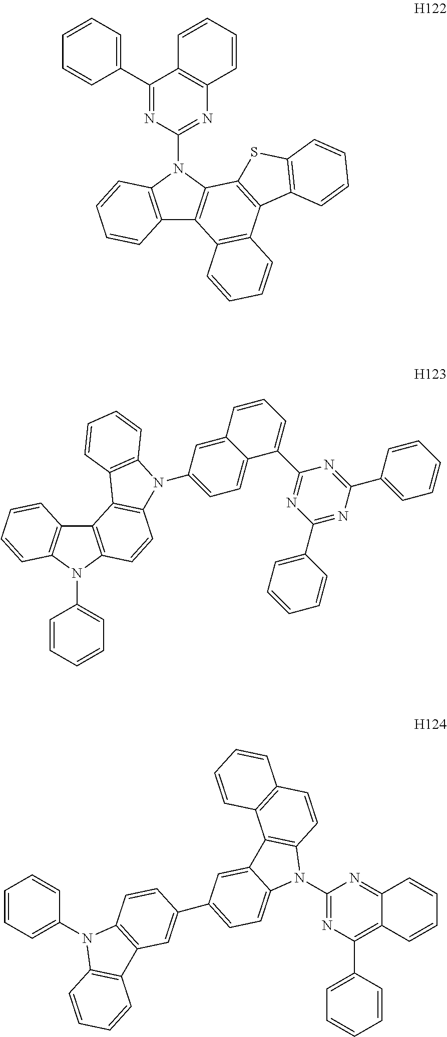 [SMILES: c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

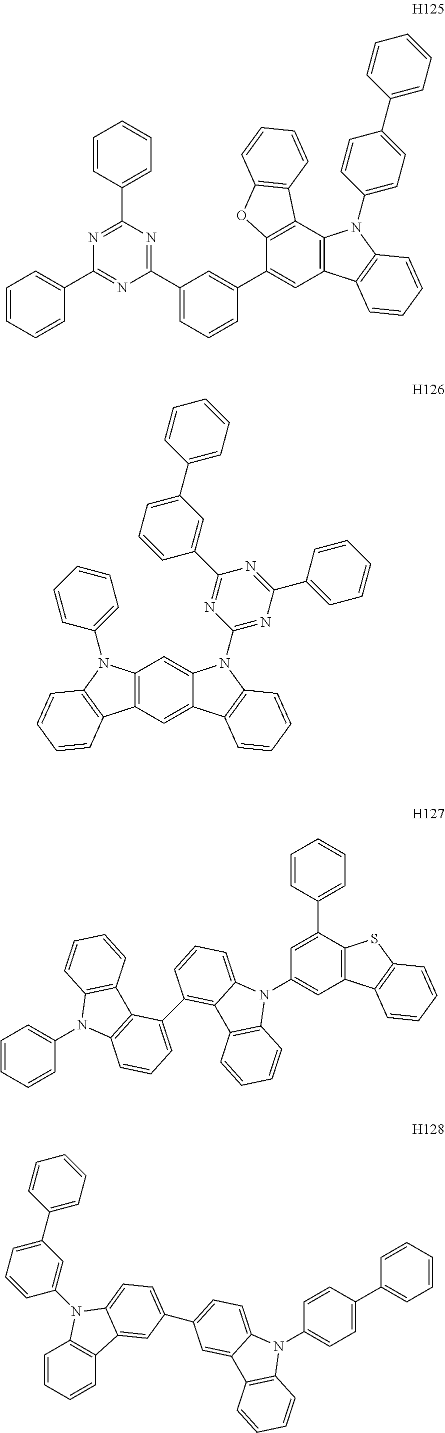 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

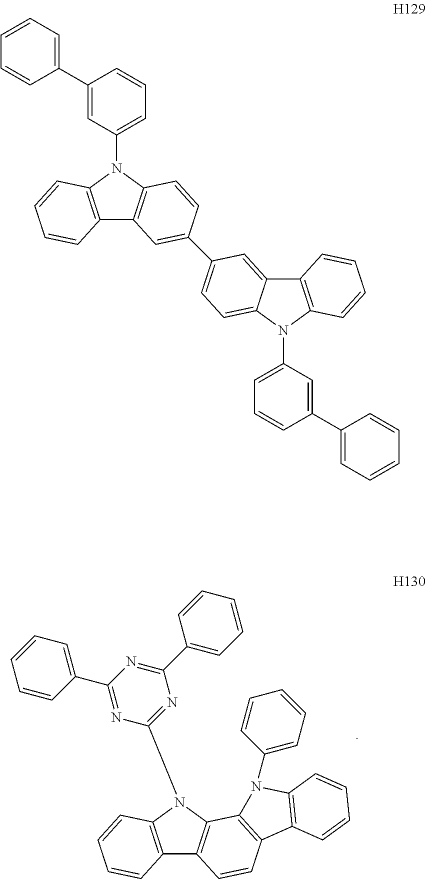 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2nc(-c3ccccc3)nc(-n3c4ccccc4c4ccc5c6ccccc6n(-c6ccccc6)c5c43)n2)cc1]

### Phosphorescent Dopant

The phosphorescent dopant may refer to the dopant described above.

According to one or more embodiments, the amount of the dopant may be in the range of 1.0 to 30 wt % (based on 100 wt % of the total weight of the emission layer). When the amount of the dopant is within the range above, the light-emitting device may have excellent or suitable efficiency and lifespan.

In one or more embodiments, the dopant may include, for example, one or more selected from among the following compounds PD26 to PD40:

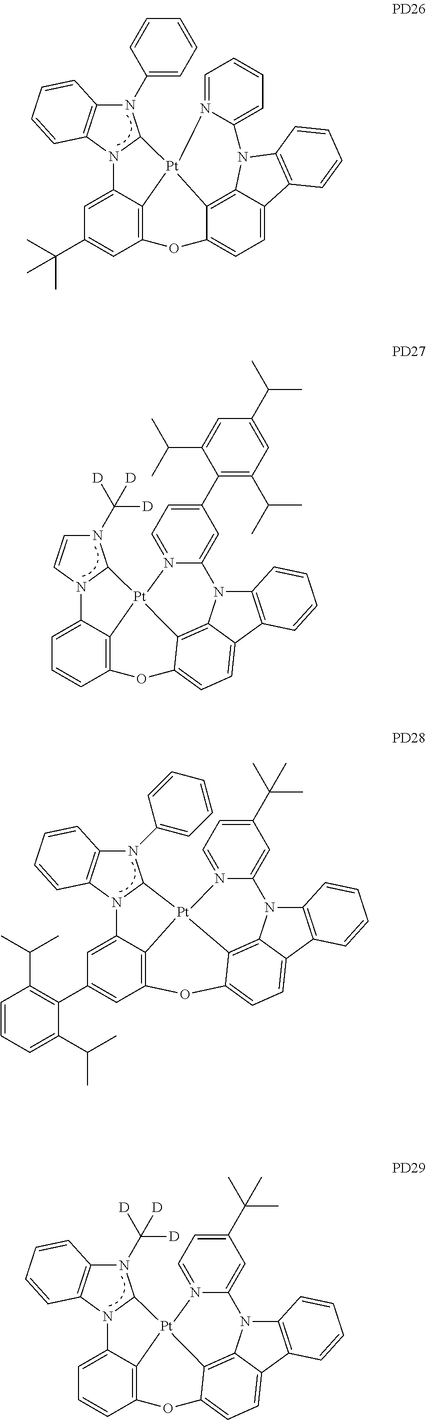

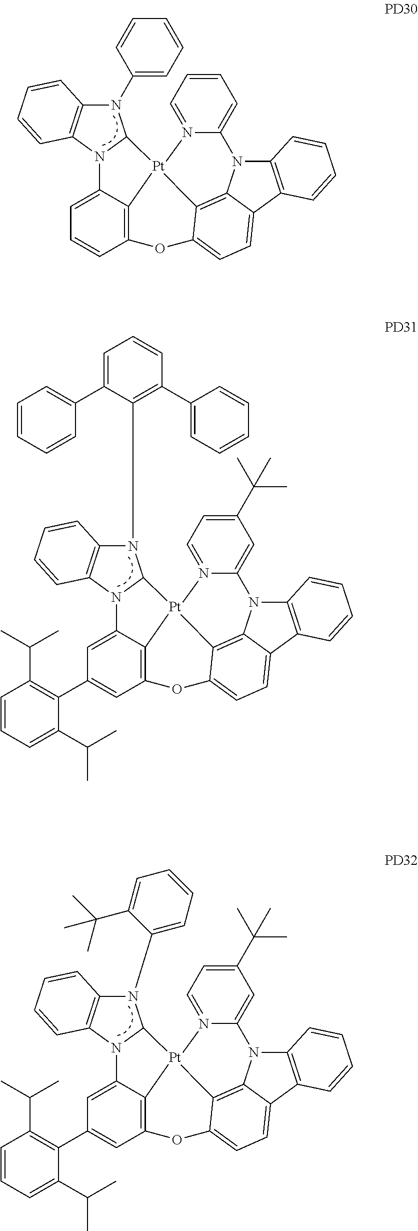

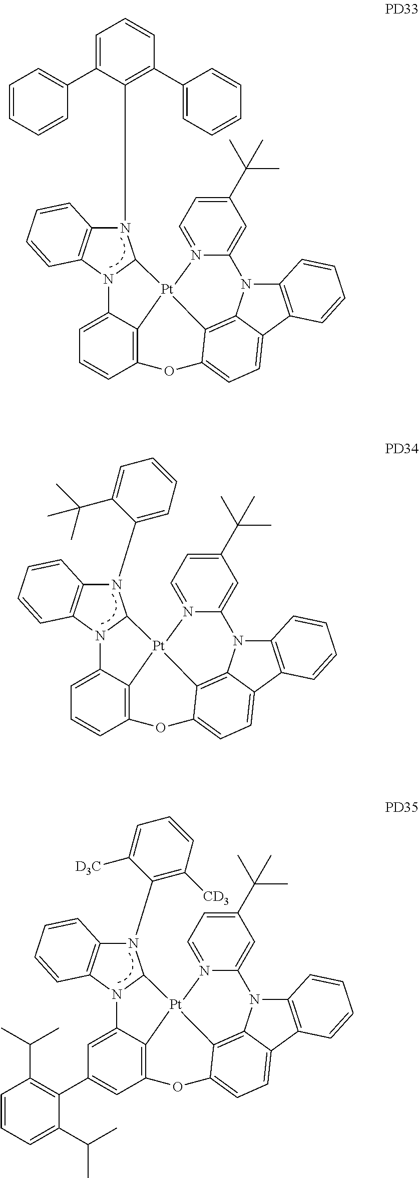

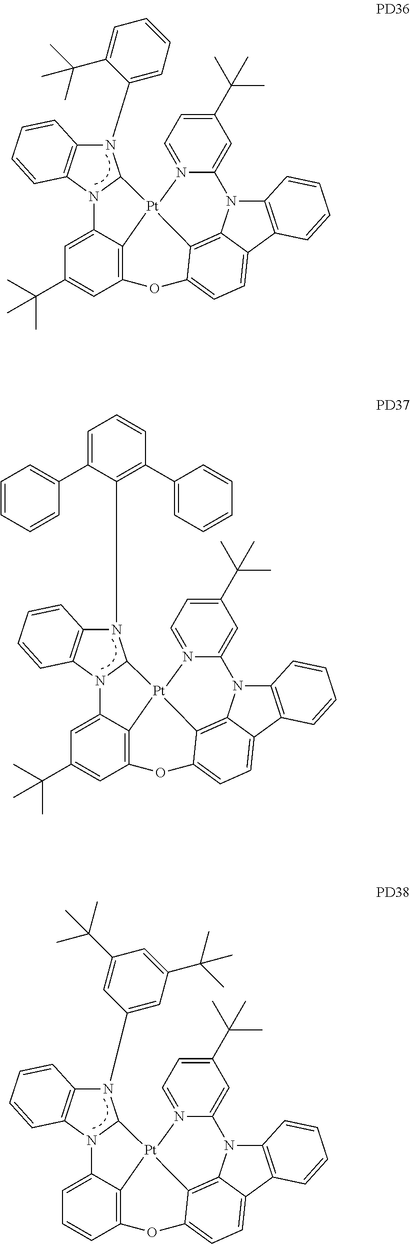

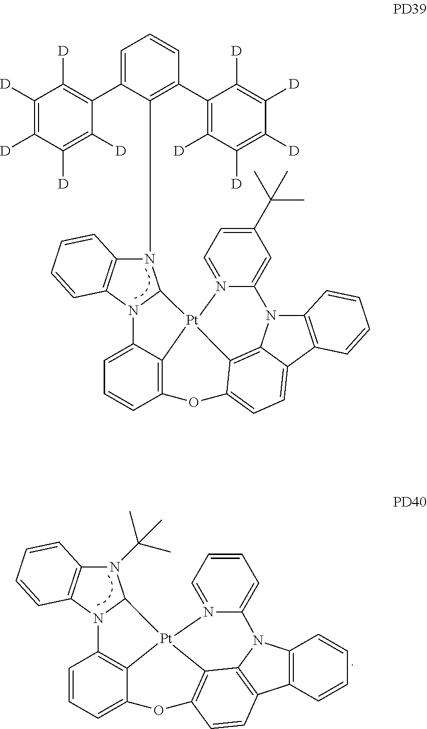

### Electron Transport Region in Interlayer 130

The electron transport region may have: i) a single-layered structure including (e.g., consisting of) a single layer including (e.g., consisting of) a single material, ii) a single-layered structure including (e.g., consisting of) a single layer including multiple different materials, or iii) a multilayer structure including multiple layers including multiple different materials.

The electron transport region may include an electron injection layer, a hole blocking layer, an electron transport layer, or any combination thereof.

In one or more embodiments, an electron transport region may be arranged between the emission layer and the charge generation layer, and the electron transport region may include a hole blocking layer, an electron transport layer, an electron injection layer, or any combination thereof. For example, in one or more embodiments, the electron transport region may have a structure including an electron transport layer/electron injection layer structure or a hole blocking layer/electron transport layer/electron injection layer structure, wherein in each structure, constituting layers are sequentially stacked from the emission layer in the stated order.

In one or more embodiments, the electron transport region (for example, a hole blocking layer or an electron transport layer in the electron transport region) may include a metal-free compound including at least one π electron-deficient nitrogen-containing C₁-C₆₀ cyclic group.

In one or more embodiments, the electron transport region may include a compound represented by Formula 601.

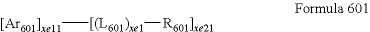

In Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be the same as described herein with respect to Q₁,
xe21 may be 1, 2, 3, 4, or 5, and
at least one selected from among Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, if (e.g., when) xe11 in Formula 601 is 2 or greater, two or more of Ar₆₀₁(s) may be linked to each other via a single bond.

In one or more embodiments, Ar₆₀₁ in Formula 601 may be a substituted or unsubstituted anthracene group.

In one or more embodiments, the electron transport region may include a compound represented by Formula 601-1:

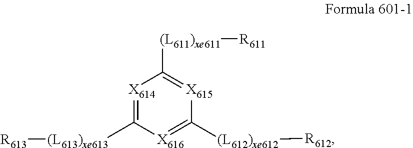

wherein, in Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each be the same as described herein with respect to L₆₀₁,
xe611 to xe613 may each be the same as described herein with respect to xe1,
R₆₁₁ to R₆₁₃ may each be the same as described herein with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

In one or more embodiments, xe1 and xe611 to xe613 in Formulae 601 and 601-1 may each independently be 0, 1, or 2.

In one or more embodiments, the electron transport domain (region) may include one of (e.g., at least one selected from among) Compounds ET1 to ET46, BCP (2,9-Dimethyl-4,7-diphenyl-1,10-phenanthroline), Bphen (4,7-Diphenyl-1,10-phenanthroline), Alq₃ (tris(8-hydroxyquinolinato)aluminum), BAlq (bis(2-methyl-8-quinolinolato-N1,O8)-(1,1′-biphenyl-4-olato)aluminum), TAZ (3-(4-biphenyl)-4-phenyl-5-tert-butylphenyl-1,2,4-triazole), NTAZ (4-(naphthalen-1-yl)-3,5-diphenyl-4H-1,2,4-triazole), or any combination thereof:

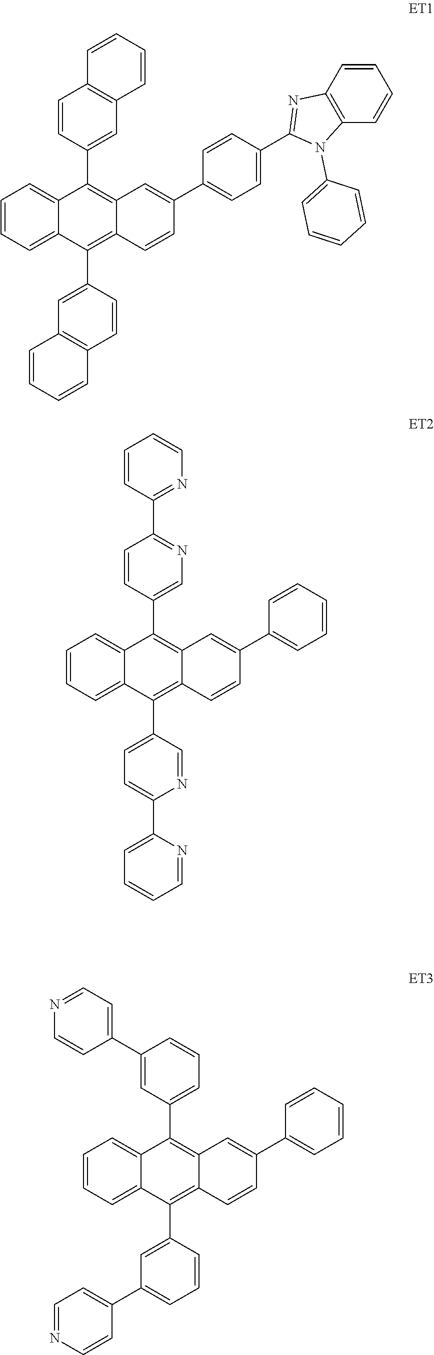 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

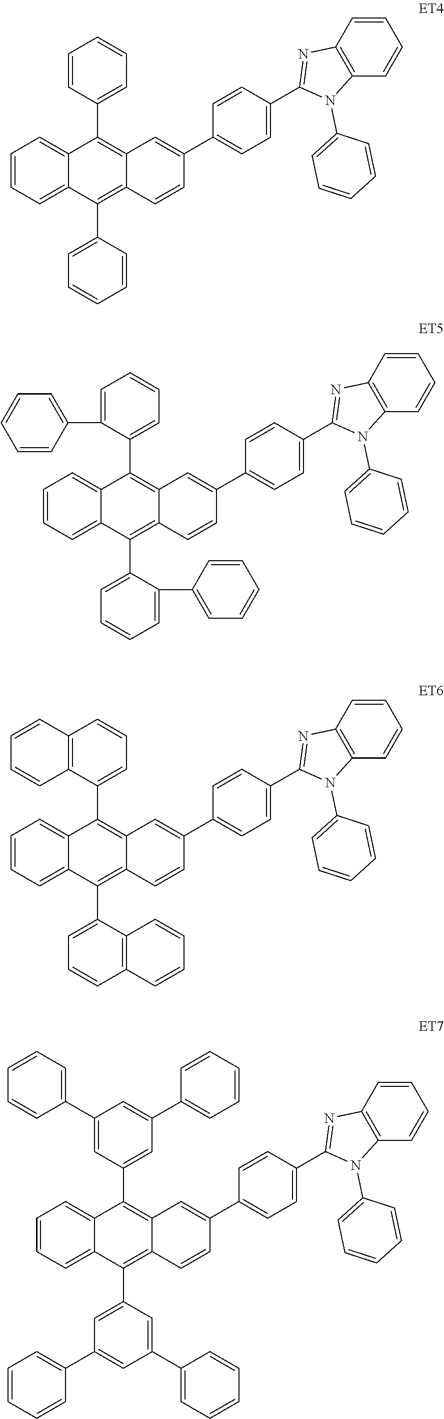 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

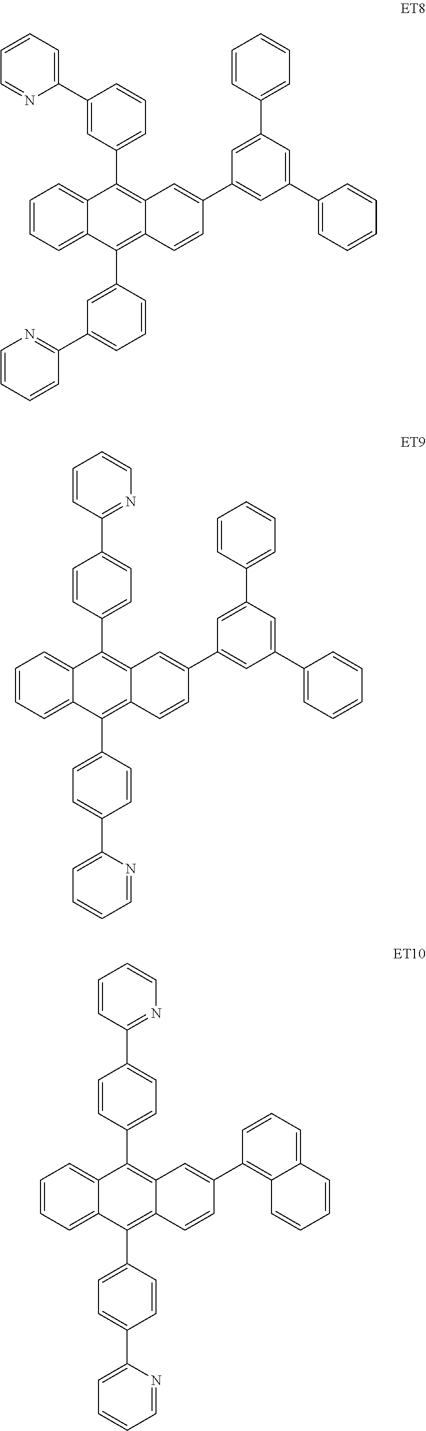 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

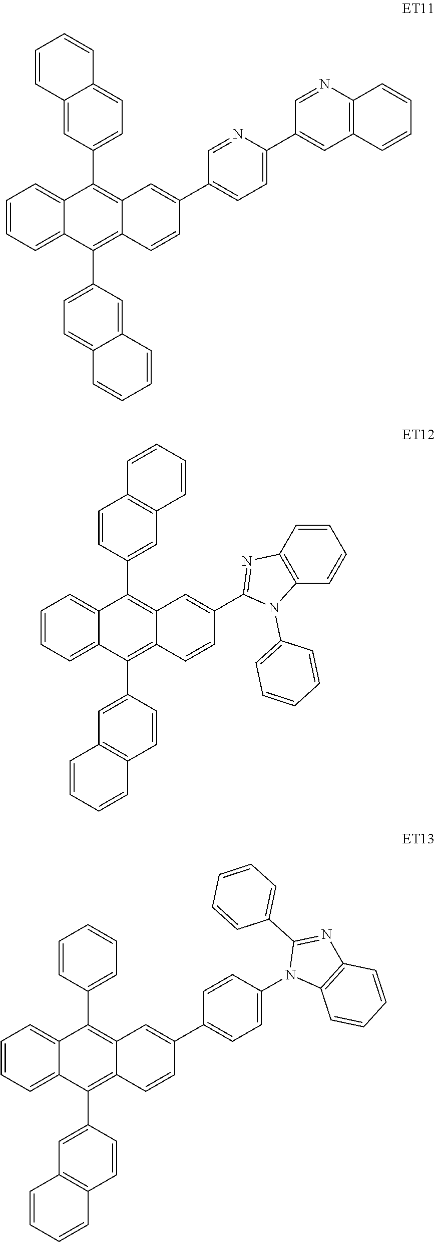 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

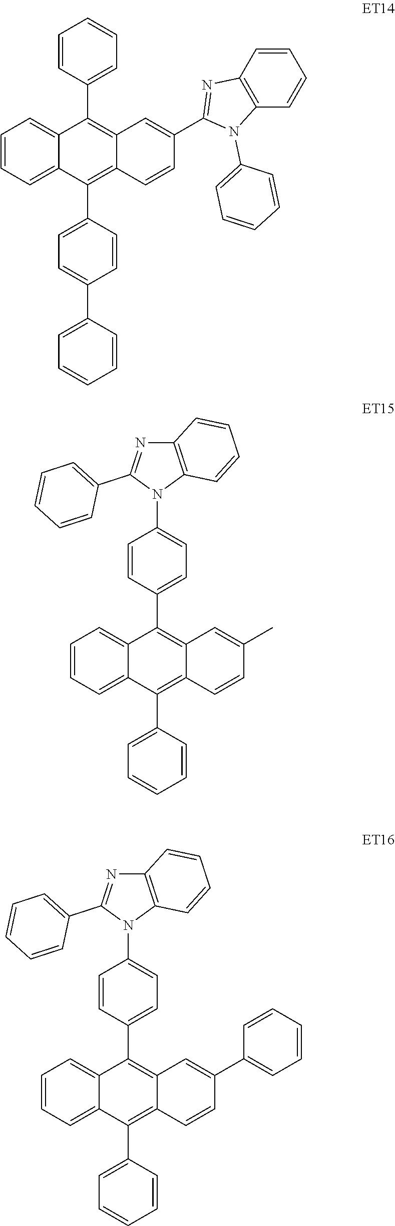 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

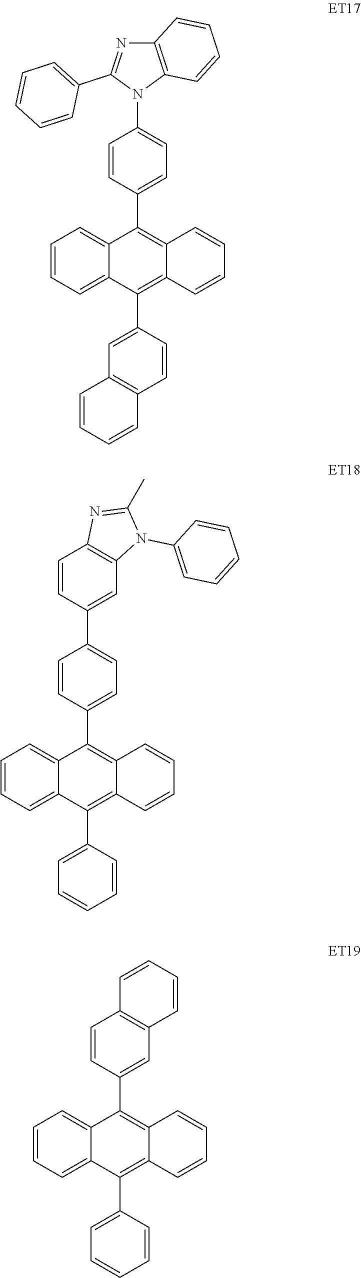

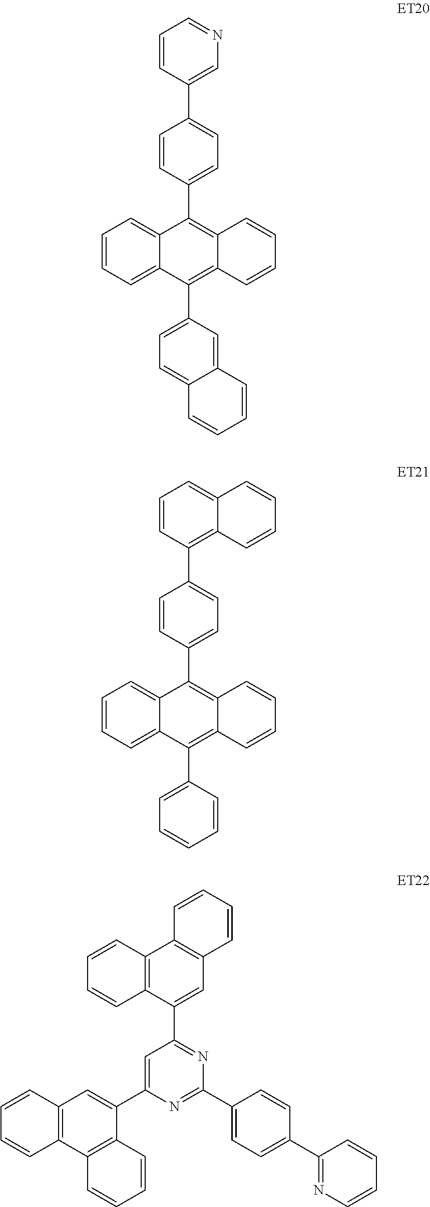

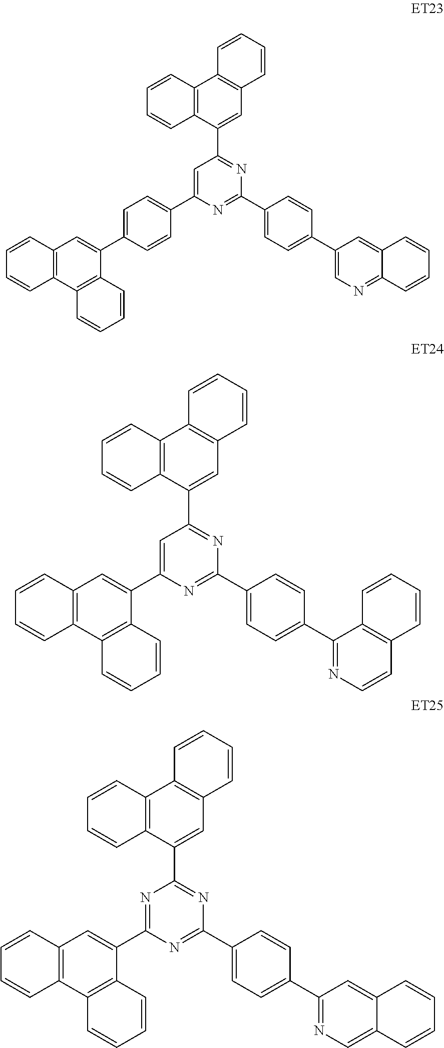 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2cc(-c3ccc(-c4nc(-c5cc6ccccc6c6ccccc56)nc(-c5cc6ccccc6c6ccccc56)n4)cc3)ncc2c1]

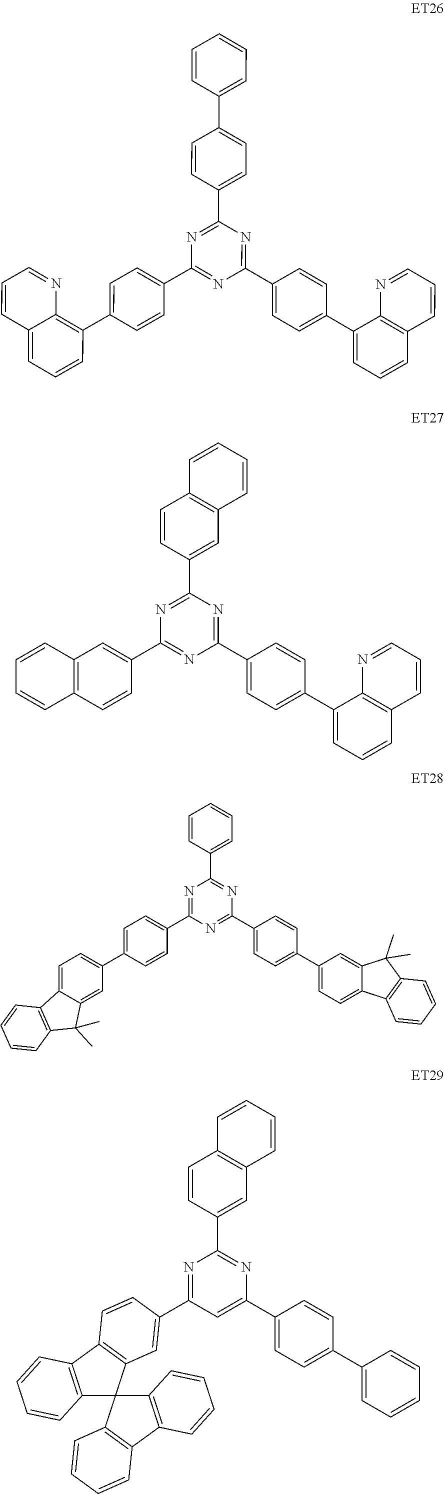 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

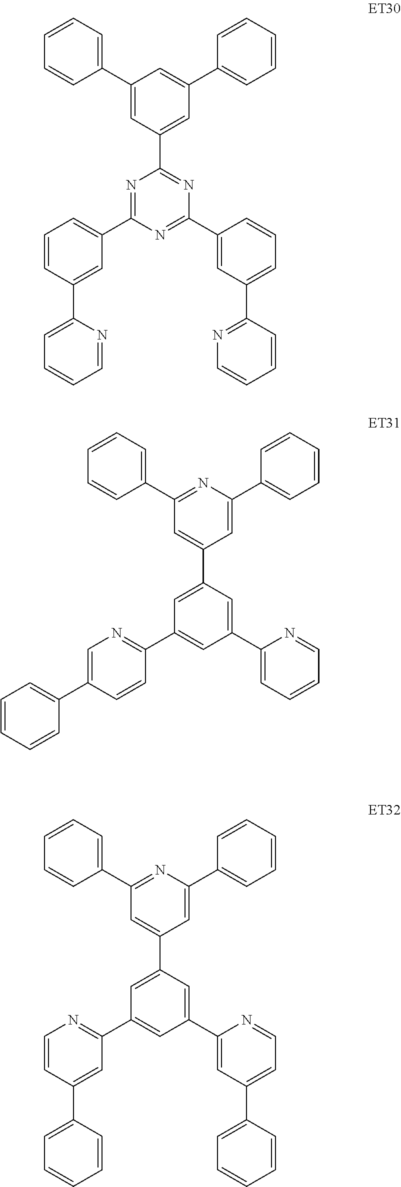 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

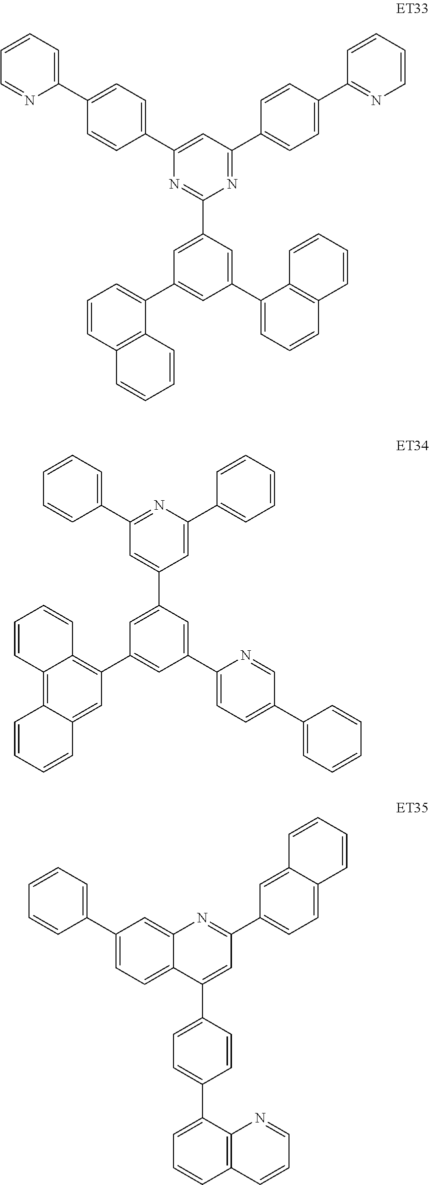 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

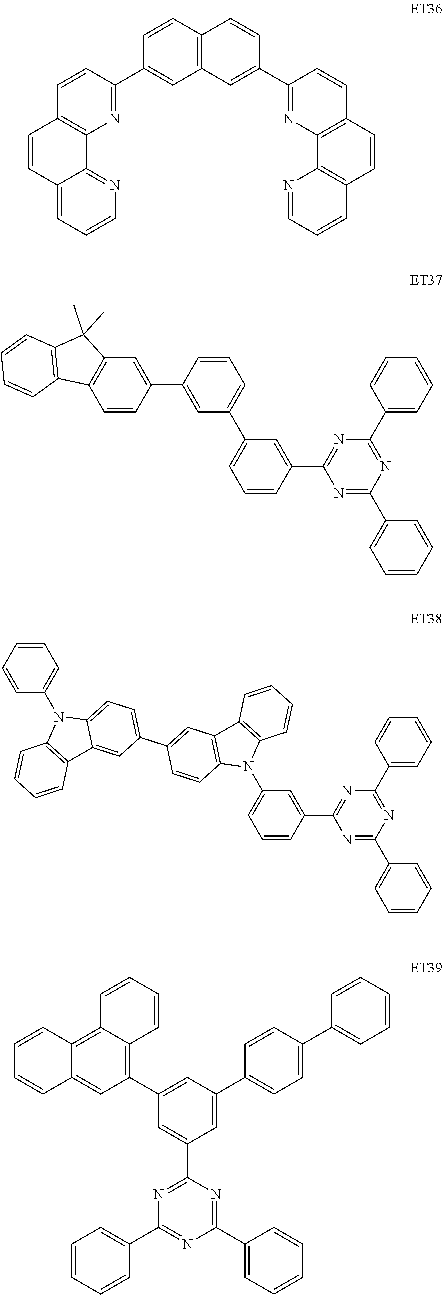 [SMILES: c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

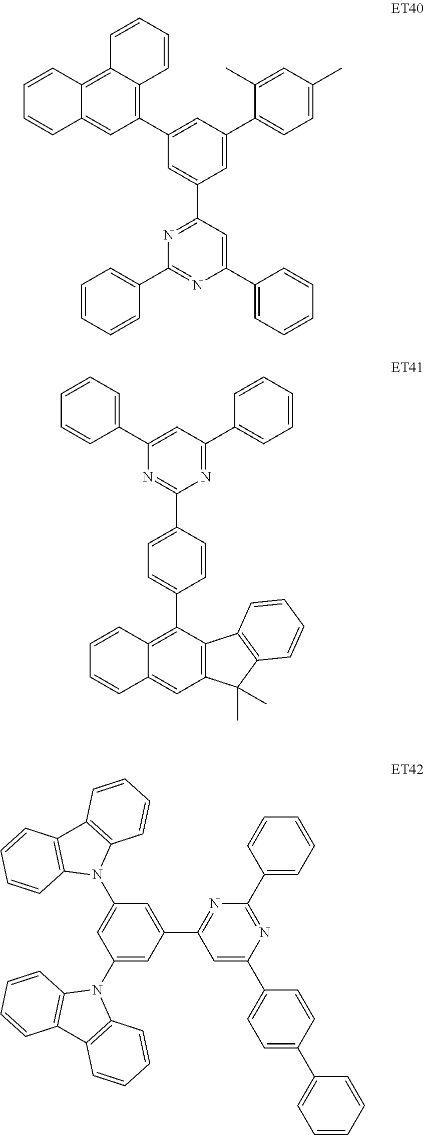 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

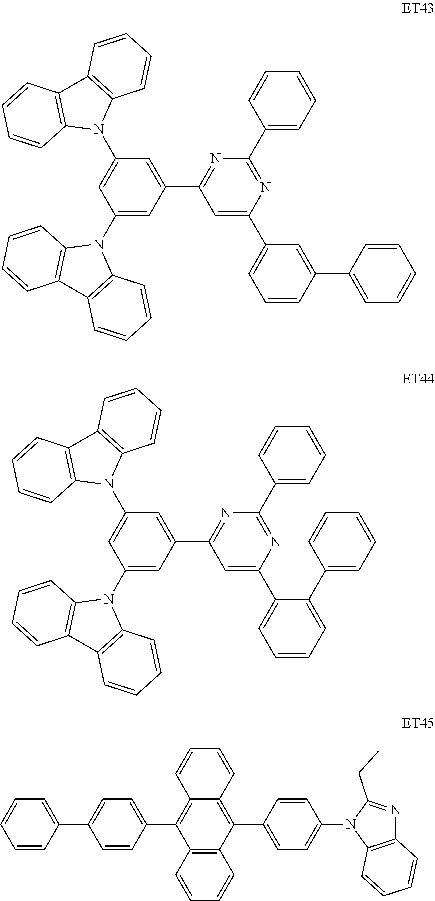 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

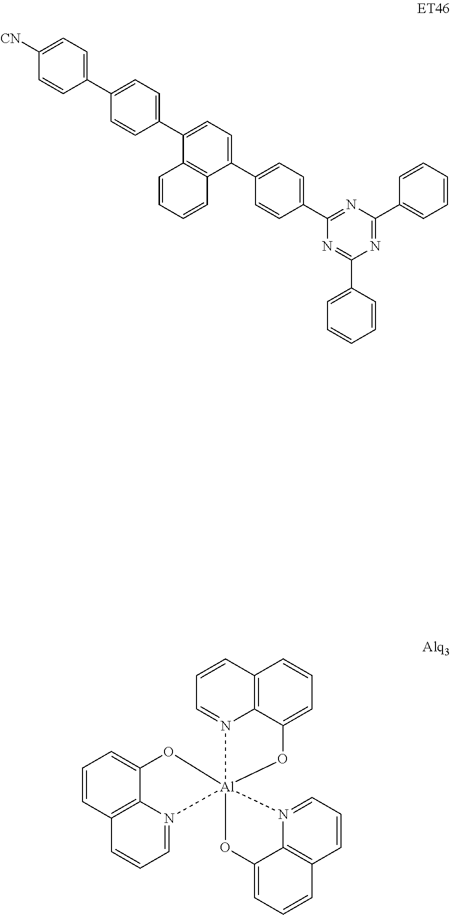

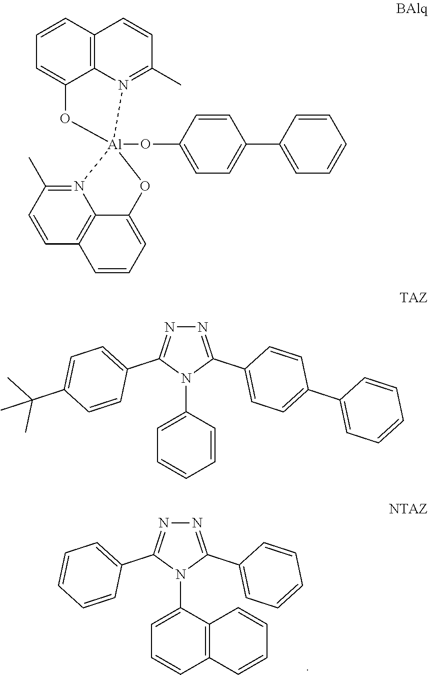

A thickness of the electron transport region may be about 100 Å to about 5,000 Å, for example, about 160 Å to about 4,000 Å. When the electron transport region includes a hole blocking layer, an electron transport layer, or any combination thereof, a thickness of the hole blocking layer may be from about 20 Å to about 1,000 Å, for example, about 30 Å to about 300 Å, and a thickness of the electron transport layer may be from about 100 Å to about 1,000 Å, for example, about 150 Å to about 500 Å. When the thicknesses of the hole-blocking layer and/or the electron transport layer are within these ranges, satisfactory electron transporting characteristics may be obtained without a substantial increase in driving voltage.

In one or more embodiments, the electron transport region (e.g., the electron transport layer in the electron transport region) may further include, in addition to one or more of the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the metal ion of the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In one or more embodiments, the metal-containing material may include a Li complex. The Li complex may include, for example, Compound ET-D1 (LiQ) or ET-D2:

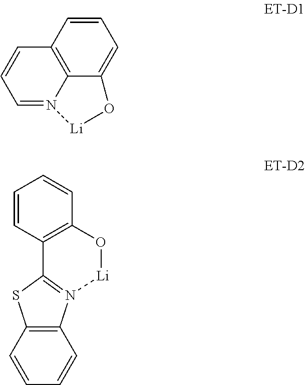

In one or more embodiments, the electron transport region may include an electron injection layer that facilitates the injection of electrons from the second electrode 150. The electron injection layer may directly contact the second electrode 150.

The electron injection layer may have: i) a single-layered structure including (e.g., consisting of) a single layer including (e.g., consisting of) a single material, ii) a single-layered structure including (e.g., consisting of) a single layer including (e.g., consisting of) multiple materials that are different from each other, or iii) a multilayer structure including multiple layers including multiple materials that are different from each other.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (for example, fluorides, chlorides, bromides, iodides, and/or the like), or tellurides of the alkali metal, the alkaline earth metal, and the rare earth metal, respectively, or any combination thereof.

The alkali metal-containing compound may include: alkali metal oxides, such as Li₂O, Cs₂O, and/or K₂O; alkali metal halides, such as LiF, NaF, CsF, KF, LiI, NaI, CsI, and/or KI; or any combination thereof. The alkaline earth metal-including compound may include an alkaline earth metal oxide, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include a lanthanide metal telluride. Non-limiting examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of metal ions of the alkali metal, one of metal ions of the alkaline earth metal, and one of metal ions of the rare earth metal, respectively, and ii) a ligand bonded to the metal ion (e.g., the respective metal ion), for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or a (e.g., any suitable) combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may include (e.g., consist of) i) an alkali metal-containing compound (e.g., alkali metal halide), or ii) a) an alkali metal-containing compound (e.g., alkali metal halide); and b) an alkali metal, an alkaline earth metal, a rare earth metal, or any combination thereof. In one or more embodiments, the electron injection layer may be a KI:Yb co-deposited layer, an RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly (e.g., substantially uniformly) or non-uniformly dispersed in a matrix including the organic material.

A thickness of the electron injection layer may be in a range of about 1 Å to about 100 Å, for example, about 3 Å to about 90 Å. When the thickness of the electron injection layer is within the range as described above, satisfactory electron injection characteristics may be obtained without a substantial increase in driving voltage.

### Second Electrode 150

The second electrode 150 may be on (e.g., arranged on) the interlayer 130 having a structure as described above. The second electrode 150 may be a cathode, which is an electron injection electrode, and as a material for forming the second electrode 150, a metal, an alloy, an electrically conductive compound, or any combination thereof, each having a low-work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a transflective electrode, or a reflective electrode.

The second electrode 150 may have a single-layer structure or a multi-layer structure including a plurality of layers.

### Capping Layer

In one or more embodiments, a first capping layer may be arranged outside (e.g., on) the first electrode 110, and/or a second capping layer may be arranged outside (e.g., on) the second electrode 150. In one or more embodiments, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

In one or more embodiments, light generated in the emission layer of the interlayer 130 of the light-emitting device 10 may be extracted toward the outside through the first electrode 110 which is a transflective electrode or a transmissive electrode, and the first capping layer. In one or more embodiments, light generated in the emission layer of the interlayer 130 of the light-emitting device 10 may be extracted toward the outside through the second electrode 150 which is a transflective electrode or a transmissive electrode, and the second capping layer.

The first capping layer and the second capping layer may increase external emission efficiency according to the principle of constructive interference. Accordingly, the light extraction efficiency of the light-emitting device 10 may be increased, and as a result, the luminescence efficiency of the light-emitting device 10 may be improved.

Each of the first capping layer and the second capping layer may include a material having a refractive index of 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and/or the second capping layer may (e.g., the first capping layer and the second capping layer may each independently) include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may each optionally be substituted with a substituent including O, N, S, Se, Si, F, Cl, Br, I, or any combination thereof. In one or more embodiments, at least one of the first capping layer and/or the second capping layer may (e.g., the first capping layer and the second capping layer may each independently) include an amine group-containing compound.

In one or more embodiments, at least one of the first capping layer and/or the second capping layer may (e.g., the first capping layer and the second capping layer may each independently) include a compound represented by Formula 201, a compound represented by Formula 202, or any combination thereof.

In one or more embodiments, at least one of the first capping layer and/or the second capping layer may (e.g., the first capping layer and the second capping layer may each independently) include one of (e.g., at least one selected from among) Compounds HT28 to HT33, one of (e.g., at least one selected from among) Compounds CP1 to CP6, β-NPB, or any combination thereof:

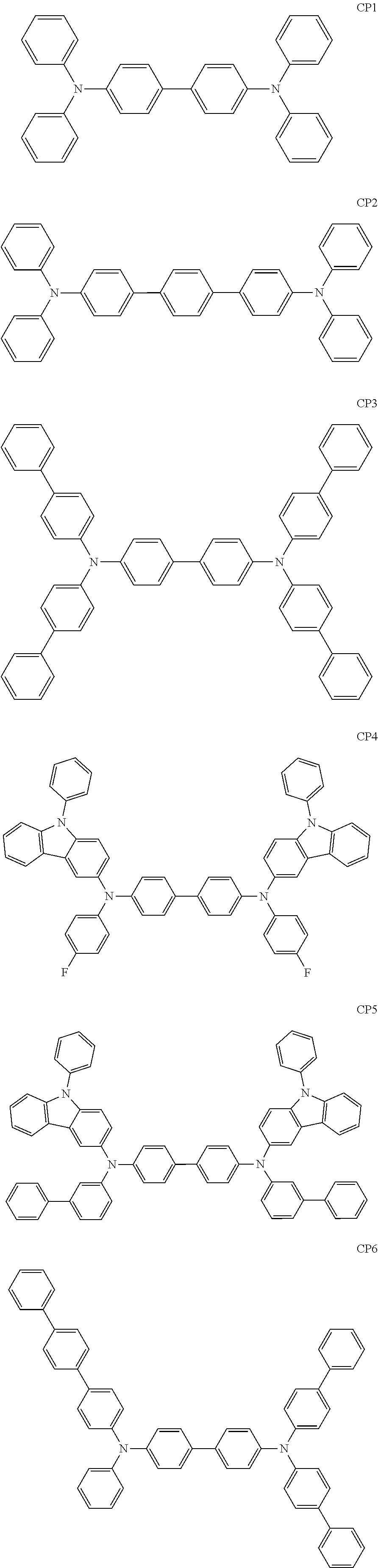

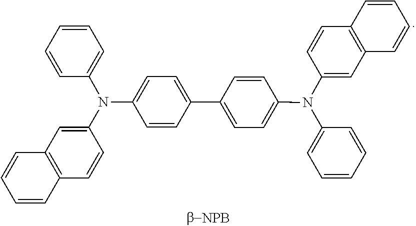 [SMILES: c1ccc(N(c2ccc(-c3ccc(N(c4ccccc4)c4ccc5ccccc5c4)cc3)cc2)c2ccc3ccccc3c2)cc1]

### Electronic Apparatus

The light-emitting device may be included in one or more suitable electronic apparatuses. For example, in one or more embodiments, the electronic apparatus including the light-emitting device may be a light-emitting apparatus, an authentication apparatus, and/or the like.

In one or more embodiments, the electronic apparatus (for example, a light-emitting apparatus) may further include, in addition to the light-emitting device, i) a color filter, ii) a color conversion layer, or iii) a color filter and a color conversion layer. The color filter and/or the color conversion layer may be arranged in at least one travel direction of light emitted from the light-emitting device. For example, in one or more embodiments, the light emitted from the light-emitting device may be blue light. Details on the light-emitting device may be referred to the descriptions provided herein. In one or more embodiments, the color conversion layer may include a quantum dot. The quantum dot may be, for example, an emissive quantum dot.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include a first area configured to emit first color light, a second area configured to emit second color light, and/or a third area configured to emit third color light, wherein the first color light, the second color light, and/or the third color light may have different maximum emission wavelengths from one another. In one or more embodiments, the first color light may be red light, the second color light may be green light, and the third color light may be blue light. In one or more embodiments, the plurality of color filter areas (or the plurality of color conversion areas) may include quantum dots. In one or more embodiments, the first area may include a red quantum dot to emit red light, the second area may include a green quantum dot to emit green light, and the third area may not include (e.g., may exclude) a (e.g., any) quantum dot. The first area, the second area, and/or the third area may each further include a scatterer.

For example, the light-emitting device may be to emit first light, the first area may be to absorb the first light to emit first-first color light, the second area may be to absorb the first light to emit second-first color light, and the third area may be to absorb the first light to emit third-first color light. In this regard, the first-first color light, the second-first color light, and the third-first color light may have different maximum emission wavelengths. For example, the first light may be blue light, the first-first color light may be red light, the second-first color light may be green light, and the third-first color light may be blue light.

In one or more embodiments, the electronic apparatus may further include a thin-film transistor, in addition to the light-emitting device as described above. The thin-film transistor may include a source electrode, a drain electrode, and an active layer, wherein one selected from among the source electrode and the drain electrode may be electrically connected to the first electrode or the second electrode of the light-emitting device.

The thin-film transistor may further include a gate electrode, a gate insulating film, and/or the like.

The active layer may include crystalline silicon, amorphous silicon, an organic semiconductor, an oxide semiconductor, and/or the like.

In one or more embodiments, the electronic apparatus may further include a sealing portion for sealing the light-emitting device. The sealing portion may be arranged between the color filter and/or the color conversion layer and the light-emitting device. The sealing portion allows light from the light-emitting device to be extracted to the outside, and concurrently (e.g., simultaneously) prevents ambient air and moisture from penetrating into the light-emitting device. The sealing portion may be a sealing substrate including a transparent glass substrate and/or a plastic substrate. The sealing portion may be a thin-film encapsulation layer including at least one layer of an organic layer and/or an inorganic layer. When the sealing portion is a thin film encapsulation layer, the electronic apparatus may be flexible.

In one or more embodiments, various functional layers may be additionally arranged on the sealing portion, in addition to the color filter and/or the color conversion layer, according to the use of the electronic apparatus. Non-limiting examples of the functional layers may include a touch screen layer and a polarizing layer. The touch screen layer may be a pressure-sensitive touch screen layer, a capacitive touch screen layer, or an infrared touch screen layer.

The authentication apparatus may further include, in addition to the light-emitting device as described above, a biometric information collector. The authentication apparatus may be, for example, a biometric authentication apparatus that authenticates an individual by using biometric information of a living body (for example, fingertips, pupils, and/or the like).

The electronic apparatus may be applied to one or more of displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

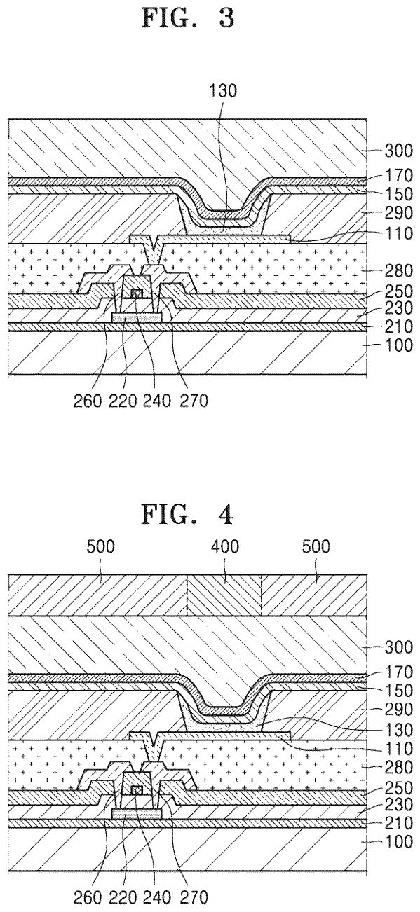

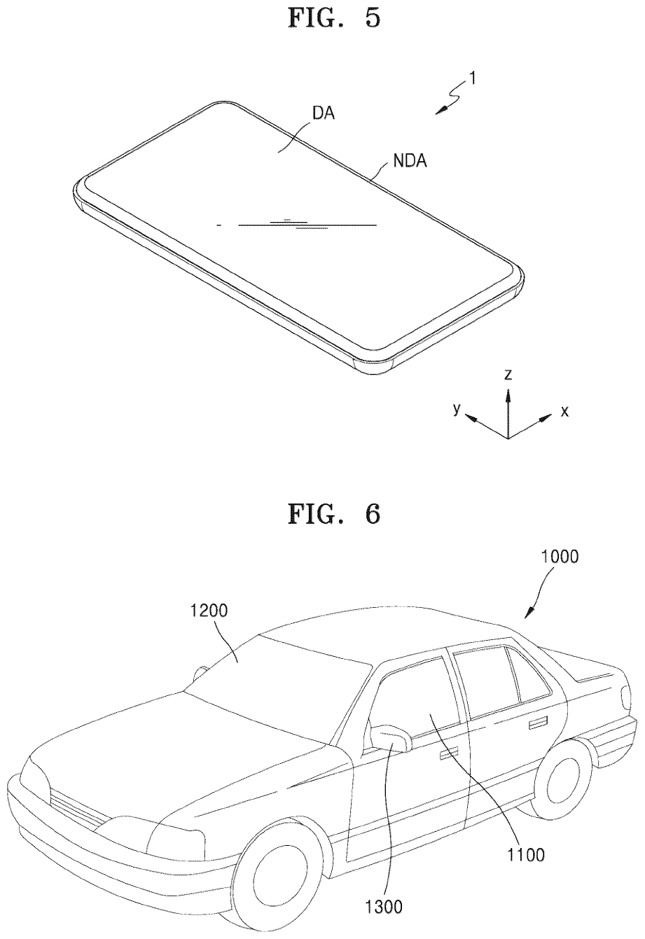

### Description of FIGS. 3 and 4

FIG. 3 is a cross-sectional view of an electronic apparatus according to one or more embodiments of the present disclosure.

The electronic apparatus of FIG. 3 may include a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be on (e.g., arranged on) the substrate 100. The buffer layer 210 may prevent or reduce penetration of impurities through the substrate 100 and may provide a flat surface on the substrate 100.

The TFT may be on (e.g., arranged on) the buffer layer 210. The TFT may include an active layer 220, a gate electrode 240, a source electrode 260, and a drain electrode 270.

The active layer 220 may include an inorganic semiconductor, such as silicon or polysilicon, an organic semiconductor, or an oxide semiconductor, and may include a source region, a drain region, and a channel region.

A gate insulating film 230 for insulating the active layer 220 from the gate electrode 240 may be arranged on the active layer 220, and the gate electrode 240 may be on (e.g., arranged on) the gate insulating film 230.

An interlayer insulating film 250 may be on (e.g., arranged on) the gate electrode 240. The interlayer insulating film 250 may be arranged between the gate electrode 240 and the source electrode 260 and between the gate electrode 240 and the drain electrode 270, to insulate these electrodes from one another.

The source electrode 260 and the drain electrode 270 may be on (e.g., arranged on) the interlayer insulating film 250. The interlayer insulating film 250 and the gate insulating film 230 may be formed to expose the source region and the drain region of the active layer 220, and the source electrode 260 and the drain electrode 270 may be arranged in contact with the exposed portions of the source region and the drain region of the active layer 220, respectively.

The TFT may be electrically connected to the light-emitting device to drive the light-emitting device, and may be covered and protected by a passivation layer 280. The passivation layer 280 may include an inorganic insulating film, an organic insulating film, or any combination thereof. The light-emitting device may be provided on the passivation layer 280. The light-emitting device may include a first electrode 110, an interlayer 130, and a second electrode 150.

The first electrode 110 may be on (e.g., arranged on) the passivation layer 280. The passivation layer 280 may be arranged to expose a portion of the drain electrode 270, not fully covering the drain electrode 270, and the first electrode 110 may be arranged to be connected to the exposed portion of the drain electrode 270.

A pixel-defining film 290 including an insulating material may be on (e.g., arranged on) the first electrode 110. The pixel-defining film 290 may expose a certain region of the first electrode 110, and the interlayer 130 may be formed in the exposed region of the first electrode 110. The pixel-defining film 290 may be a polyimide-based organic film or a polyacrylic-based organic film. In one or more embodiments, at least some layers of the interlayer 130 may extend beyond the upper portion of the pixel defining layer 290 and may thus be located in the form of a common layer.

The second electrode 150 may be on (e.g., arranged on) the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be arranged on the light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic-based resin (for example, polymethyl methacrylate, polyacrylic acid, and/or the like), an epoxy-based resin (for example, aliphatic glycidyl ether (AGE), and/or the like), or any combination thereof; and/or a (e.g., any suitable) combination of the inorganic film and the organic film.

FIG. 4 is a cross-sectional view of an electronic apparatus according to one or more embodiments of the present disclosure.

The electronic apparatus of FIG. 4 is substantially the same as the electronic apparatus of FIG. 3, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the electronic apparatus of FIG. 4 may be a tandem light-emitting device.

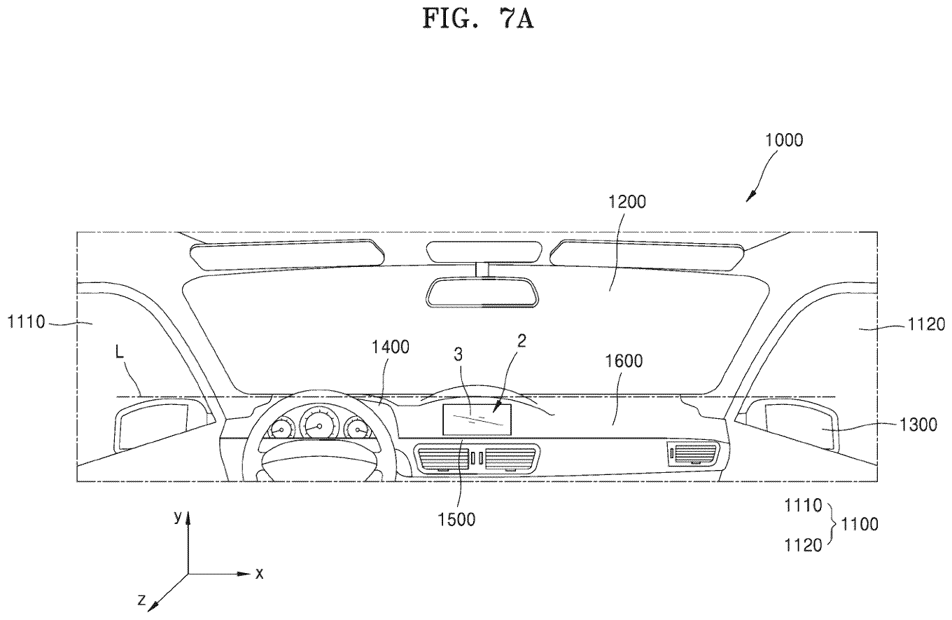

### Description of FIG. 5

FIG. 5 is a schematic perspective view of electronic equipment 1 including a light-emitting device according to one or more embodiments of the present disclosure. The electronic equipment 1 may be, as an apparatus that displays a moving image or a still image, a portable electronic equipment, such as a mobile phone, a smartphone, a tablet personal computer (PC), a mobile communication terminal, an electronic notebook, an electronic book, a portable multimedia player (PMP), a navigation, or an ultra-mobile PC (UMPC), as well as one or more suitable products, such as a television, a laptop, a monitor, a billboard, or an Internet of things (IoT) device. The electronic equipment 1 may be such a product above or a part thereof. In addition, the electronic equipment 1 may be a wearable device, such as a smart watch, a watch phone, a glasses-type (kind) display, or a head mounted display (HMD), or a part of the wearable device. In one or more embodiments, the electronic equipment may be at least one of a flat panel display, a curved display, a computer monitor, a medical monitor, a television, an advertisement board, an indoor light, an outdoor light, a signaling light, a head-up display, a fully transparent display, a partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a 3D display, a virtual reality display, an augmented reality display, a vehicle, a video wall including multiple displays tiled together, a theater screen, a stadium screen, a phototherapy device, or a signboard. However, embodiments of the present disclosure are not limited thereto. In one or more embodiments, the electronic equipment 1 may be a dashboard of a vehicle, a center information display (CID) arranged on a center fascia or dashboard of a vehicle, a room mirror display instead of a side-view mirror of a vehicle, an entertainment for the back seat of a vehicle, or a display arranged on the back of the front seat of a vehicle, a head up display (HUD) installed on the front of a vehicle or projected on a front window glass thereof, or a computer generated hologram augmented reality head up display (CGH AR HUD). FIG. 5 illustrates one or more embodiments in which the electronic equipment 1 is a smartphone for convenience of explanation.

The electronic equipment 1 may include a display area DA and a non-display area NDA outside (e.g., around) the display area DA. A display apparatus of the electronic equipment 1 may implement an image through an array of a plurality of pixels that are two-dimensionally arranged in the display area DA.

The non-display area NDA is an area that does not display an image, and may entirely be around (e.g., surround) the display area DA. On the non-display area NDA, a driver for providing electrical signals or power to display devices arranged on the display area DA may be arranged. On the non-display area NDA, a pad, which is an area to which an electronic element or a printed circuit board may be electrically connected, may be arranged.

In the electronic equipment 1, a length in an x-axis direction and a length (e.g., a width) in a y-axis direction may be different from each other. In one or more embodiments, as shown in FIG. 4, the length in the x-axis direction may be less than the length (e.g., the width) in the y-axis direction. In one or more embodiments, the length in the x-axis direction may be substantially the same as the length (e.g., the width) in the y-axis direction. In one or more embodiments, the length in the x-axis direction may be greater than the length (e.g., the width) in the y-axis direction.

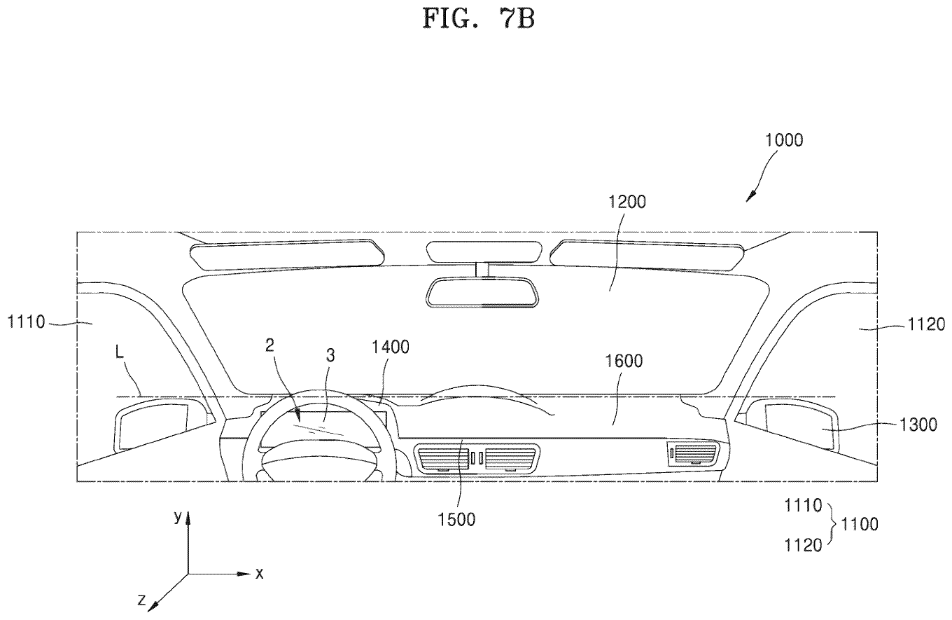

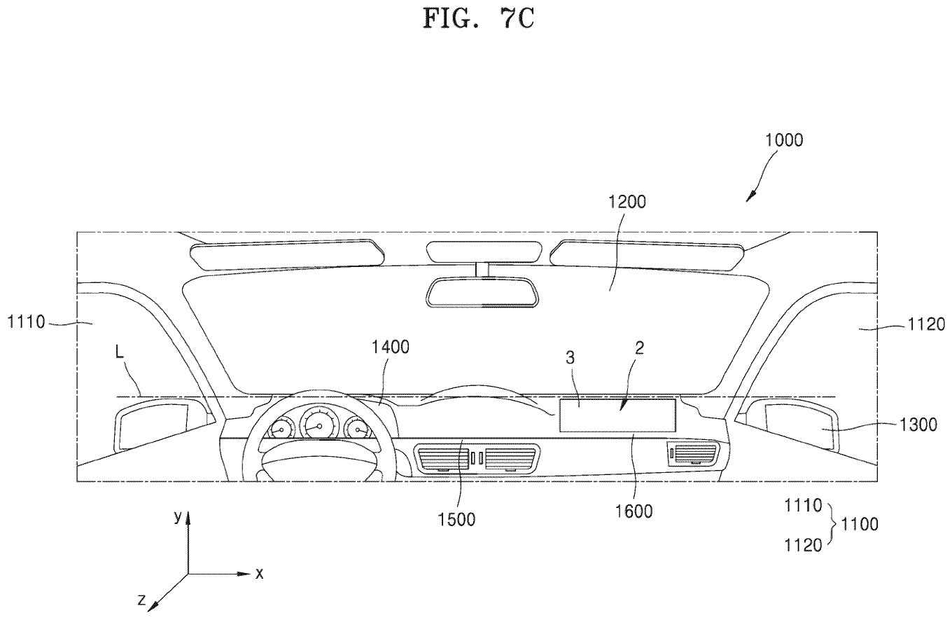

### Descriptions of FIGS. 6 and 7A to 7C

FIG. 6 is a diagram illustrating an exterior of a vehicle 1000 as electronic equipment including a light-emitting device according to one or more embodiments of the present disclosure. FIGS. 7A to 7C are each a schematic view of an interior of the vehicle 1000 according to one or more embodiments.

Referring to FIGS. 6 and 7A to 7C, the vehicle 1000 may refer to one or more suitable apparatuses for moving an object to be transported, such as a human, an object, or an animal, from a departure point to a destination point. The vehicle 1000 may include a vehicle traveling on a road or a track, a vessel moving over the sea or a river, an airplane flying in the sky using the action of air, and/or the like.

In one or more embodiments, the vehicle 1000 may travel on a road or a track. The vehicle 1000 may move in a certain direction according to rotation of at least one wheel thereof. In one or more embodiments, the vehicle 1000 may include a three-wheeled or four-wheeled vehicle, a construction machine, a two-wheeled vehicle, a prime mover device, a bicycle, or a train running on a track.

The vehicle 1000 may include a body having an interior and an exterior, and a chassis in which mechanical apparatuses necessary for driving are installed as other parts except for the body of the vehicle 1000. The exterior of the body of the vehicle 1000 may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side-view mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on a side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced and/or apart (e.g., spaced apart or separated) from each other in an x direction or a −x direction (the direction opposite the x-direction). In one or more embodiments, the first side window glass 1110 and the second side window glass 1120 may be spaced and/or apart (e.g., spaced apart or separated) from each other in the x direction or the −x direction. For example, an imaginary straight line L connecting the side window glasses 1100 may extend in the x direction or the −x direction. In one or more embodiments, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 opposite to (e.g., facing) each other.

The side-view mirror 1300 may provide a rear view of the vehicle 1000. The side-view mirror 1300 may be installed on the exterior of the body of the vehicle. In one or more embodiments, a plurality of side-view mirrors 1300 may be provided. Any one of the plurality of side-view mirrors 1300 may be arranged outside the first side window glass 1110. Another of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge, a turn signal indicator, a high beam indicator, a warning light, a seat belt warning light, an odometer, a tachograph, an automatic shift selector indicator, a door open warning light, an engine oil warning light, and/or a low fuel warning light.

The center fascia 1500 may include a control panel on which a plurality of buttons for adjusting an audio device, an air conditioning device, and/or a seat heater are arranged. The center fascia 1500 may be arranged on one side of the cluster 1400.

The passenger seat dashboard 1600 may be spaced and/or apart (e.g., spaced apart or separated) from the cluster 1400, and the center fascia 1500 may be arranged between the cluster 1400 and the passenger seat dashboard 1600. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be arranged inside the vehicle 1000. In one or more embodiments, the display apparatus 2 may be arranged between the side window glasses 1100 opposite to (e.g., facing) each other. The display apparatus 2 may be arranged on at least one of the cluster 1400, the center fascia 1500, or the passenger seat dashboard 1600.

The display apparatus 2 may include an organic light-emitting display apparatus, an inorganic electroluminescent display apparatus, a quantum dot display apparatus, and/or the like. Hereinafter, as the display apparatus 2 according to one or more embodiments, an organic light-emitting display apparatus including the light-emitting device of the present disclosure will be described as an example, but one or more suitable types (kinds) of display apparatuses as described above may be used in embodiments.

Referring to FIG. 7A, in one or more embodiments, the display device 2 may be arranged on the center fascia 1500. In one or more embodiments, the display apparatus 2 may display navigation information. In one or more embodiments, the display apparatus 2 may display information regarding audio settings, video setting, and/or vehicle settings.

Referring to FIG. 7B, in one or more embodiments, the display device 2 may be arranged on the cluster 1400. In these embodiments, the cluster 1400 may display driving information and/or the like through the display apparatus 2. For example, the cluster 1400 may be digitally implemented. The digital cluster 1400 may display vehicle information and driving information in an image. In one or more embodiments, a needle and a gauge of a tachometer and one or more suitable warning light icons may be displayed by a digital signal.

Referring to FIG. 7C, in one or more embodiments, the display device 2 may be arranged on the passenger seat dashboard 1600. The display apparatus 2 may be embedded in the passenger seat dashboard 1600 or arranged on the passenger seat dashboard 1600. In one or more embodiments, the display apparatus 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information different from information displayed on the cluster 1400 and/or information displayed on the center fascia 1500.

### Manufacturing Method

Respective layers included in the hole transport region, the emission layer, and respective layers included in the electron transport region may be each formed in a certain region by using one or more suitable methods selected from among vacuum deposition, spin coating, casting, Langmuir-Blodgett (LB) deposition, ink-jet printing, laser-printing, laser-induced thermal imaging, and/or the like.

When the layers constituting the hole transport region, the emission layer, and the layers constituting the electron transport region are formed by vacuum deposition, the deposition may be performed at a deposition temperature in a range of about 100° C. to about 500° C., at a vacuum degree in a range of about 10⁻⁸ torr to about 10⁻³ torr, and at a deposition speed in a range of about 0.01 Å/sec to about 100 Å/sec, depending on a material to be included in a layer to be formed and the structure of a layer to be formed.

When layers constituting the hole transport region, the emission layer, and layers constituting the electron transport region are formed by spin coating, the spin coating may be performed at a coating speed of about 2,000 rpm to about 5,000 rpm and at a heat treatment temperature of about 80° C. to about 200° C. by taking into account a material to be included in a layer to be formed and the structure of a layer to be formed.

### Definition of Terms

The term “C₃-C₆₀ carbocyclic group” as used herein refers to a cyclic group including (e.g., consisting of) carbon atoms as the only ring-forming atoms and having three to sixty carbon atoms, and the term “C₁-C₆₀ heterocyclic group” as used herein refers to a cyclic group that has one to sixty carbon atoms and further includes, in addition to carbon atom(s), a heteroatom as a ring-forming atom. The C₃-C₆₀ carbocyclic group and the C₁-C₆₀ heterocyclic group may each be a monocyclic group including (e.g., consisting of) one (exactly one) ring or a polycyclic group in which two or more rings are condensed with each other. In one or more embodiments, the number of ring-forming atoms of the C₁-C₆₀ heterocyclic group may be 3 to 61.

The term “cyclic group” as used herein may include both (e.g., simultaneously) the C₃-C₆₀ carbocyclic group and the C₁-C₆₀ heterocyclic group.

The term “π electron-rich C₃-C₆₀ cyclic group” as used herein refers to a cyclic group that has three to sixty carbon atoms and does not include *—N═*′ as a ring-forming moiety, and the term “π electron-deficient nitrogen-containing C₁-C₆₀ cyclic group” as used herein refers to a heterocyclic group that has one to sixty carbon atoms and includes *—N═*′ as a ring-forming moiety.

In one or more embodiments,
the C₃-C₆₀ carbocyclic group may be i) Group T1 or ii) a condensed cyclic group in which two or more of Group T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more of Group T2 are condensed with each other, or iii) a condensed cyclic group in which at least one Group T2 and at least one Group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like),
the π electron-deficient nitrogen-containing C₁-C₆₀ cyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,
Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
Group T3 may a furan group, a thiophene group, a 1H-pyrrole group, a silole group, or a borole group,
Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ cyclic group” as used herein may refer to a group condensed to any cyclic group, a monovalent group, or a polyvalent group (for example, a divalent group, a trivalent group, a tetravalent group, and/or the like) according to the structure of a formula for which the corresponding term is used. In one or more embodiments, “a benzene group” may be a benzo group, a phenyl group, a phenylene group, and/or the like, which may be easily understand by one of ordinary skill in the art according to the structure of a formula including the “benzene group.”

Non-limiting examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Non-limiting examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has 1 to 60 carbon atoms, and non-limiting examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond in the middle or at the terminus of a C₂-C₆₀ alkyl group, and non-limiting examples thereof include an ethenyl group, a propenyl group, and a butenyl group. The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of a C₂-C₆₀ alkyl group, and non-limiting examples thereof may include an ethynyl group, a propynyl group, and/or the like. The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by —OA₁₀₁ (wherein A₁₀₁ is a C₁-C₆₀ alkyl group), and non-limiting examples thereof include a methoxy group, an ethoxy group, and an isopropyloxy group.

The term “C₃-C₁₀ cycloalkyl group” as used herein refers to a monovalent saturated hydrocarbon monocyclic group including 3 to 10 carbon atoms. Non-limiting examples of the C₃-C₁₀ cycloalkyl group as used herein include a cyclopropyl group, a cyclobutyl group, a cyclopentyl group, a cyclohexyl group, a cycloheptyl group, a cyclooctyl group, an adamantyl group, a norbornyl (bicyclo[2.2.1]heptyl) group, a bicyclo[1.1.1]pentyl group, a bicyclo[2.1.1]hexyl group, and a bicyclo[2.2.2]octyl group. The term “C₃-C₁₀ cycloalkylene group” as used herein refers to a divalent group having substantially the same structure as the C₃-C₁₀ cycloalkyl group.

The term “C₁-C₁₀ heterocycloalkyl group” as used herein refers to a monovalent cyclic group of 1 to 10 carbon atoms, further including, in addition to carbon atom(s), at least one heteroatom, as ring-forming atom(s), and non-limiting examples thereof include a 1,2,3,4-oxatriazolidinyl group, a tetrahydrofuranyl group, and a tetrahydrothiophenyl group. The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent cyclic group that has 3 to 10 carbon atoms, at least one carbon-carbon double bond in the ring thereof, and no aromaticity, and non-limiting examples thereof may include a cyclopentenyl group, a cyclohexenyl group, a cycloheptenyl group, and/or the like. The term “C₃-C₁₀ cycloalkenylene group” as used herein refers to a divalent group having substantially the same structure as the C₃-C₁₀ cycloalkenyl group.

The term “C₁-C₁₀ heterocycloalkenyl group” as used herein refers to a monovalent cyclic group of 1 to 10 carbon atoms, further including, in addition to carbon atom(s), at least one heteroatom, as ring-forming atom(s), and having at least one double bond in the cyclic structure thereof. Non-limiting examples of the C₁-C₁₀ heterocycloalkenyl group may include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having substantially the same structure as the C₁-C₁ heterocycloalkenyl group.

The term “C₆-C₆₀ aryl group” as used herein refers to a monovalent group having a carbocyclic aromatic system of 6 to 60 carbon atoms, and the term “C₆-C₆₀ arylene group” as used herein refers to a divalent group having a carbocyclic aromatic system of 6 to 60 carbon atoms. Non-limiting examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having a heterocyclic aromatic system of 1 to 60 carbon atoms, further including, in addition to carbon atom(s), at least one heteroatom, as ring-forming atom(s). The term “C₁-C₆₀ heteroarylene group” as used herein refers to a divalent group having a heterocyclic aromatic system of 1 to 60 carbon atoms, further including, in addition to carbon atom(s), at least one heteroatom, as ring-forming atom(s). Non-limiting examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure when considered as a whole. Non-limiting examples of the monovalent non-aromatic condensed polycyclic group may include an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein refers to a divalent group having substantially the same structure as the monovalent non-aromatic condensed polycyclic group.

The term “monovalent non-aromatic condensed heteropolycyclic group” as used herein refers to a monovalent group (for example, having 1 to 60 carbon atoms) having two or more rings condensed to each other, further including, in addition to carbon atom(s), at least one heteroatom, as ring-forming atom(s), and having no aromaticity in its entire molecular structure when considered a whole. Non-limiting examples of the monovalent non-aromatic condensed heteropolycyclic group may include a pyrrolyl group, a thiophenyl group, a furanyl group, an indolyl group, a benzoindolyl group, a naphtho indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and/or the like. The term “divalent non-aromatic condensed heteropolycyclic group” as used herein refers to a divalent group having substantially the same structure as the monovalent non-aromatic condensed heteropolycyclic group.

The term “C₆-C₆₀ aryloxy group” as used herein refers to —OA₁₀₂ (wherein A₁₀₂ is a C₆-C₆₀ aryl group), and the term “C₆-C₆₀ arylthio group” as used herein refers to —SA₁₀₃ (wherein A₁₀₃ is a C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, cyano group, a C₁-C₆₀ alkyl group, a C₁-C₆₀ alkoxy group, a phenyl group, a biphenyl group, or any combination thereof.

The term “heteroatom” as used herein refers to any atom other than a carbon atom or a hydrogen atom. Non-limiting examples of the heteroatom may include O, S, N, P, Si, B, Ge, Se, or any combination thereof.

The term “transition metal” as used herein may include Hf, Ta, W, Re, Os, Ir, Pt, Au, and/or the like.

The term “Ph” as used herein refers to a phenyl group, the term “Me” as used herein refers to a methyl group, the term “Et” as used herein refers to an ethyl group, the term “tert-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” For example, the “biphenyl group” is a substituted phenyl group having a C₆-C₆₀ aryl group as a substituent.

The term “terphenyl group” as used herein refers to “a phenyl group substituted with a biphenyl group”. For example, the “terphenyl group” is a substituted phenyl group having, as a substituent, a C₆-C₆₀ aryl group substituted with a C₆-C₆₀ aryl group.

The number of carbon atoms in the substituent definition is example. For example, in the C₁-C₆₀ alkyl group, the number of carbon atoms, 60, is an example, and the definition for the alkyl group is equally applied to the C₁-C₂₀ alkyl group.

For example, the number of carbon atoms in a C₃-C₆₀ carbocyclic group is example, and the definition for a carbocyclic group applies equally to a C₅ carbocyclic group and a C₄-C₆ carbocyclic group. The other cases may each independently be the same.

Any hydrogen in the compound structures described herein may optionally be substituted with deuterium.

* and *′ as used herein, unless defined otherwise, each refer to a bonding site to a neighboring atom in a corresponding formula.

Hereinafter, compounds and light-emitting devices according to one or more embodiments will be described in more detail with reference to the following Examples.

### EXAMPLES

### Synthesis Example 1 Compound 6

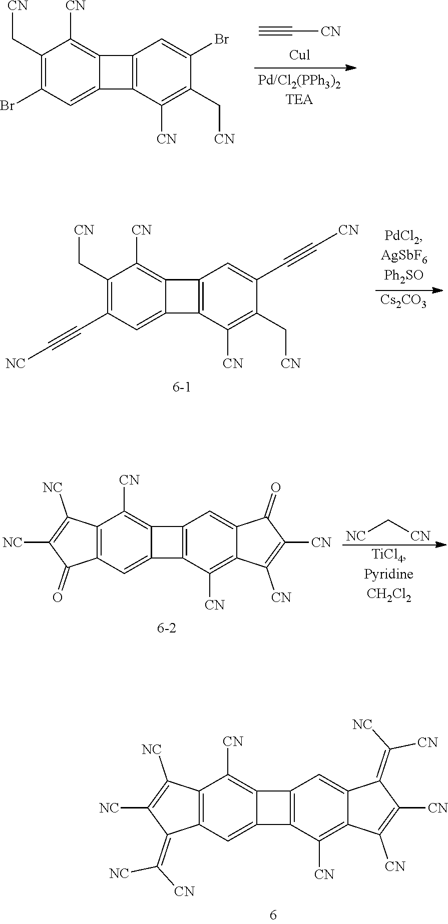

### Synthesis of Intermediate 6-1

2,2′-(3,7-dibromobiphenylene-2,6-diyl)diacetonitrile (10 g, 0.026 mol) and propiolonitrile (2.76 g, 0.054 mol) were dissolved in 100 mL of tetrahydrofuran (THF), and CuI (0.49 g, 0.003 mol) was added and stirred for 1 hour. Trimethylamine (TEA) (0.26 g, 0.003 mol) and PdCl₂(PPh₃)₂ (1.8 g, 0.003 mol) were additionally added and stirred for 3 hours. The solvent was removed under reduced pressure and the residue was purified by column chromatography to obtain 7.02 g of intermediate 6-1. (Yield: 83%)

### Synthesis of Intermediate 6-2

Intermediate 6-1 (7 g, 0.021 mol) and PdCl₂ (0.76 g, 0.004 mol), AgSbF₆ (2.2 g, 0.006 mol), and Ph₂SO (25.87 g, 0.13 mol) were dissolved in 1,2-chloroethane, and the mixture was refluxed at 60° C. for 24 hours, then Cs₂CO₃ (14.6 g, 0.045 mol) was added and refluxed for another 12 hours. After the reaction was completed, extraction was performed with CH₂Cl₂, the solvent was removed under reduced pressure, 15 mL of HCl was added, and the mixture was stirred for 2 hours. After extraction with CH₂Cl₂ and NH₄Cl aqueous solutions, moisture in organic layer was removed with anhydrous MgSO₄. After removing the solvent under reduced pressure, the residue was purified by column chromatography to obtain 2.85 g of intermediate 6-2. (Yield: 37.5%) Synthesis of Compound 6

Intermediate 6-2 (2.5 g, 0.007 mol) and malononitrile (3.71 g, 0.056 mol) were added to 300 mL of anhydrous CH₂Cl₂ and stirred for 1 hour in an inert gas atmosphere. TiCl₄ (10.6 g, 0.056 mol) and pyridine (8.9 g, 0.112 mol) were further added sequentially and stirred for 2 hours. After the reaction was completed by addition of water, extraction was performed with CH₂Cl₂. The extract was washed with brine and moisture was removed with anhydrous MgSO₄, and solvent was removed under reduced pressure to obtain a residue. After dissolving the residue in a small amount of CH₂Cl₂, 2.22 g of compound 6 was obtained by purifying with a silica filter. (Yield: 70%).

### Synthesis Example 2 Compound 11

Compound 11 was synthesized using substantially the same method as that described in Synthesis Example 1 for the synthesis of compound 6, except that 3,7-dibromo-2,6-bis(cyanomethyl)biphenylene-1,5-dicarbonitrile was used instead of 2,2′-(3,7-dibromobiphenylene-2,6-diyl)diacetonitrile in the synthesis process of intermediate 6-1 and that ethyne was used instead of propiolonitrile. (1.96 g, Yield: 61.7%) Synthesis Example 3 Compound 30

Compound 30 was synthesized using substantially the same method as that described in Synthesis Example 1 for the synthesis of compound 6, except that 2,2′-(3,6-dibromobiphenylene-2,7-diyl)diacetonitrile was used instead of 2,2′-(3,7-dibromobiphenylene-2,6-diyl)diacetonitrile in the synthesis process of intermediate 6-1. (1.75 g, Yield: 55%)

### Synthesis Example 4 Compound 35

Compound 35 was synthesized using substantially the same method as that described in Synthesis Example 1 for the synthesis of compound 6, except that 2,7-dibromo-3,6-bis(cyanomethyl)biphenylene-1,5-dicarbonitrile was used instead of 2,2′-(3,7-dibromobiphenylene-2,6-diyl)diacetonitrile in the synthesis process of intermediate 6-1 and that ethyne was used instead of propiolonitrile. (1.88 g, Yield: 59.2%)

The proton nuclear magnetic resonance spectroscopy (¹H NMR) and mass spectroscopy/fast atom bombardment (MS/FAB) data of each of the above compounds are shown in Table 1.

TABLE 1

| | | MS/FAB | |
|---|---|---|---|
| Compound | ¹H NMR (CDCl₃, 500 MHz) | found | calc. |
| 6 | 7.66 (s, 4H) | 452.01 | 452.06 |
| 11 | 7.96 (s, 2H), 7.45 (s, 2H) | 451.98 | 452.06 |
| 30 | 7.60 (s, 4H) | 451.92 | 452.06 |
| 35 | 7.89 (s, 2H), 7.39 (s, 2H) | 452.02 | 452.06 |

The highest occupied molecular orbital (HOMO) energy, lowest unoccupied molecular orbital (LUMO) energy, hole mobility, electron mobility, and glass transition temperature of each of Compounds 6, 11, 30 and 35 were measured, and the results are shown in Table 2.

Evaluation method of HOMO energy level: Using cyclic voltammetry (CV) (electrolyte: 0.1 M Bu₄NPF₆/solvent: DMF (dimethylformamide)/electrode: 3-electrode system (working electrode: glassy carbon (GC), reference electrode: Ag/AgCl, auxiliary electrode: Pt)), a potential (V)-current (A) graph of each compound was obtained, and the HOMO energy level of each compound was calculated from the oxidation onset of the graph.

Evaluation method of LUMO energy level: Using cyclic voltammetry (CV) (electrolyte: 0.1 M Bu₄NPF₆/solvent: DMF (dimethylformamide)/electrode: 3-electrode system (working electrode: GC, reference electrode: Ag/AgCl, auxiliary electrode: Pt)), a potential (V)-current (A) graph of each compound was obtained, and the LUMO energy level of each compound was calculated from the reduction onset of the graph.

Evaluation method of hole mobility and electron mobility: Evaluated using the space-charge-limited current (SCLC) method described in literature “‘Hole mobility of N,N′-bis(naphthanlen-1-yl)-N,N′-bis(phenyl)benzidine investigated by using space-charge-limited currents,’ Appl. Phys. Lett. 90, 203512 (2007)”, the entire content of which is incorporated herein by reference.

TABLE 2

| | | | Hole | | Glass |
|---|---|---|---|---|---|
| | | | mobility | Electron | transition |
| | HOMO | LUMO | (cm²/ | mobility | temperature |
| Compound | (eV) | (eV) | (Vs)) | (cm²/(Vs)) | (° C.) |
| 6 | −7.24 | −5.35 | 6.84E−03 | 3.46E−02 | 127.6 |
| 11 | −7.33 | −5.22 | 2.47E−03 | 5.84E−02 | 132.4 |
| 30 | −7.22 | −5.35 | 1.28E−03 | 4.08E−02 | 127.9 |
| 35 | −7.33 | −5.21 | 3.02E−03 | 5.37E−02 | 127.3 |

### Fabrication of Light-Emitting Device

### Example 1

As an anode, a glass substrate (product of Corning Inc.) with a 15 Ω/cm² (1,200 Å) ITO electrode formed thereon was cut to a size of 50 mm×50 mm×0.7 mm, sonicated with isopropyl alcohol and then with pure water each for 5 minutes, cleaned by irradiation of ultraviolet rays and exposure of ozone thereto for 30 minutes, and then mounted on a vacuum deposition apparatus.

Compound 6 as a p-dopant and compound HT3 were vacuum deposited on the anode at a weight ratio of 3:97 to form a hole injection layer with a thickness of 100 Å, and then compound HT40 was deposited on the hole injection layer to form a hole transport layer with a thickness of 1250 Å.

Compound H129 as a first host, compound H130 as a second host, and compound PD40 as a dopant were co-deposited at a weight ratio of 45:45:10 on the hole transport layer to form an emission layer with a thickness of 300 Å.

Compound ET37 was deposited on the emission layer to form a hole-blocking layer with a thickness of 50 Å, then compound ET46 and LiQ were co-deposited at a weight ratio of 5:5 on the hole-blocking layer to form an electron-transporting layer with a thickness of 310 Å, then Yb was deposited on the electron-transporting layer to form an electron-injection layer with a thickness of 15 Å, and then Ag and Mg were co-deposited at a weight ratio of 5:5 on the electron-injection layer to form a cathode with a thickness of 1000 Å, thereby fabricating a light-emitting device.

### Examples 2 to 4

A light-emitting device was fabricated in substantially the same manner as in Example 1, except that each of the compounds in Table 3 was used as a p-dopant.

### Comparative Examples 1 to 3

A light-emitting device was fabricated in substantially in the same manner as in Example 1, except that each of the compounds in Table 3 was used as a p-dopant.

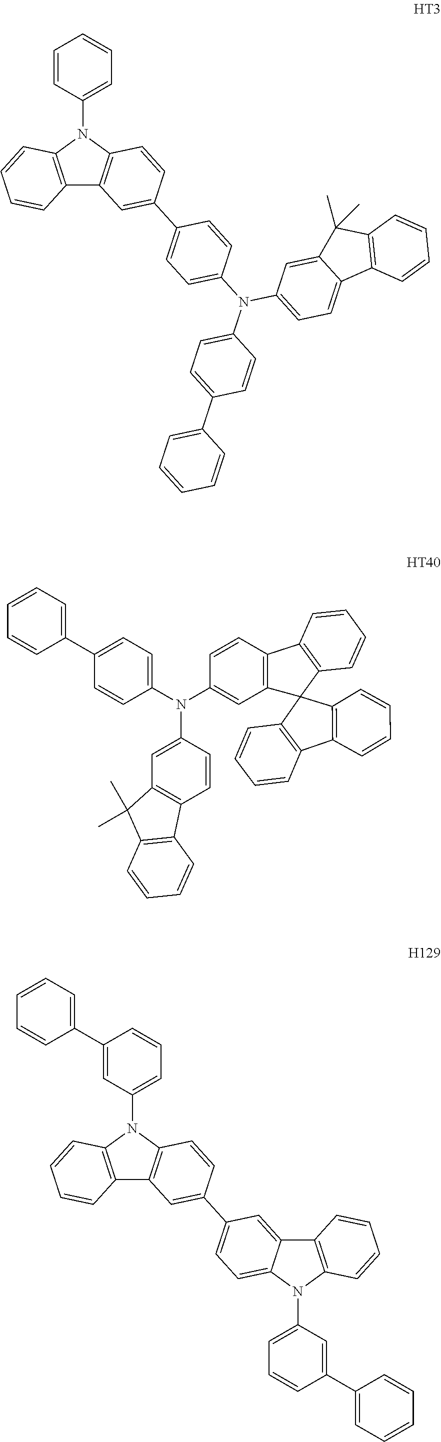 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2cccc(-n3c4ccccc4c4cc(-c5ccc6c(c5)c5ccccc5n6-c5cccc(-c6ccccc6)c5)ccc43)c2)cc1]

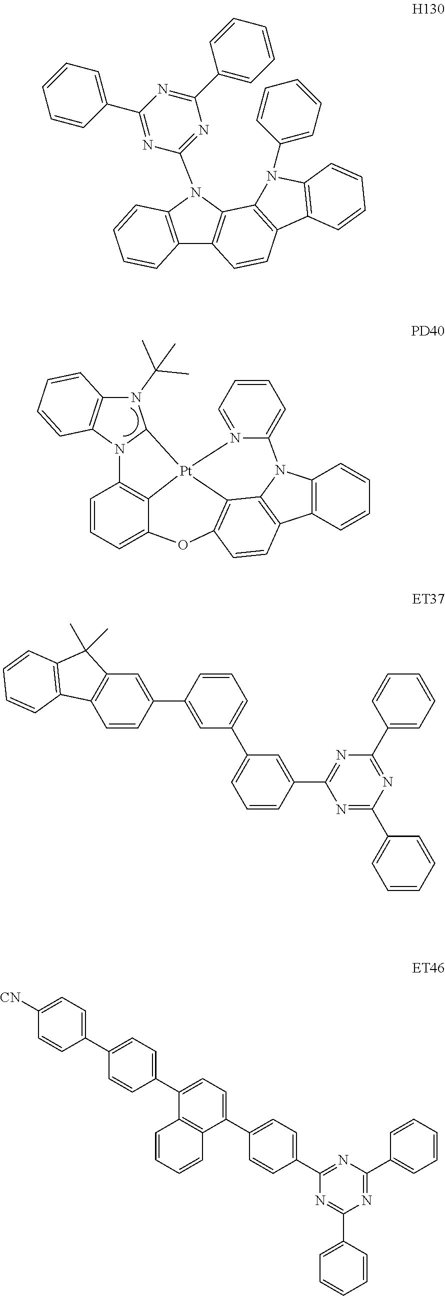

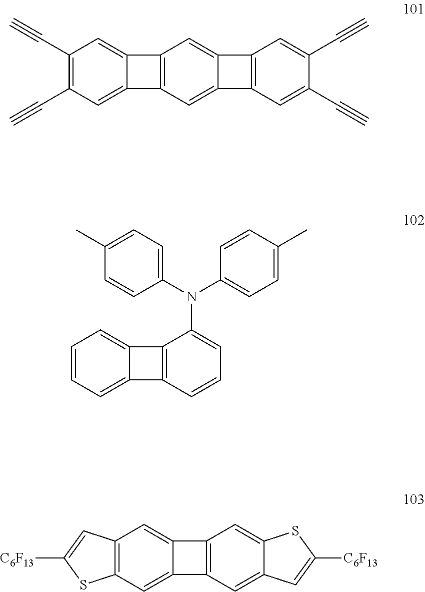

The results of each of the light-emitting devices are shown in Table 3.

The driving voltage of the light-emitting device was measured using a source meter (Keithley Instrument, 2400 series), and the current efficiency was measured using a luminance meter CS-2000 (Konica Minolta). In addition, in order to measure the device lifespan, the time required to reach 95% of the initial luminance was measured for each of Examples 1 to 4 and Comparative Examples 1 to 3, and a relative lifespan was calculated by comparing with Comparative Example 1.

TABLE 3

| | | Driving | Current | |
|---|---|---|---|---|
| | | voltage | efficiency | Relative |
| | Compound | (V) | (cd/A) | lifespan |
| Example 1 | 6 | 3.3 | 16.9 | 250% |
| Example 2 | 11 | 3.4 | 16.9 | 249% |
| Example 3 | 30 | 3.4 | 16.9 | 250% |
| Example 4 | 35 | 3.5 | 16.9 | 249% |
| Comparative | 101 | 4.4 | 12.0 | 100% |
| Example 1 | | | | |
| Comparative | 102 | 4.2 | 12.9 | 133% |
| Example 2 | | | | |
| Comparative | 103 | 6.2 | 9.2 | 104% |
| Example 3 | | | | |

Referring to Table 3, it can be seen that the efficiency, driving voltage, and lifespan of each of the Examples are superior to those of the Comparative Examples.

The light-emitting device according to one or more embodiments of the present disclosure has superior driving voltage, efficiency and lifespan compared to comparative light-emitting devices in the comparable art. For example, Example 1 demonstrates a light-emitting device fabricated utilizing Compound 6 as a p-dopant, which exhibits a driving voltage of 3.3 V, a current efficiency of 16.9 cd/A, and a relative lifespan of 250%. Example 2 utilizes Compound 11 as a p-dopant, resulting in a light-emitting device with a driving voltage of 3.4 V, a current efficiency of 16.9 cd/A, and a relative lifespan of 249%. Example 3 features Compound 30 as a p-dopant, achieving a driving voltage of 3.4 V, a current efficiency of 16.9 cd/A, and a relative lifespan of 250%. Example 4 employs Compound 35 as a p-dopant, with the resulting light-emitting device exhibiting a driving voltage of 3.5 V, a current efficiency of 16.9 cd/A, and a relative lifespan of 249%.

In contrast, Comparative Example 1 shows a light-emitting device with a driving voltage of 4.4 V, a current efficiency of 12.0 cd/A, and a relative lifespan of 100%. Comparative Example 2 and Comparative Example 3 also demonstrate inferior performance metrics, with higher driving voltages and lower current efficiencies and lifespans compared to the examples of the present disclosure. These results illustrate that the light-emitting devices fabricated utilizing the compounds disclosed in the present application exhibit significantly improved driving voltage, efficiency, and lifespan, making them highly advantageous for practical applications in electronic devices.

In the present disclosure, it will be understood that the term “comprise(s)/comprising,” “include(s)/including,” or “have/has/having” specifies the presence of stated features, integers, steps, operations, elements, and/or components, but do not preclude the presence or addition of one or more other features, integers, steps, operations, elements, components, and/or groups thereof. Additionally, the terms “comprise(s)/comprising,” “include(s)/including,” “have/has/having”, or other similar terms include or support the terms “consisting of” and “consisting essentially of,” indicating the presence of stated features, integers, steps, operations, elements, and/or components, without or essentially without the presence of other features, integers, steps, operations, elements, components, and/or groups thereof.

In the context of the present application and unless otherwise defined, the terms “use,” “using,” and “used” may be considered synonymous with the terms “utilize,” “utilizing,” and “utilized,” respectively.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in the present disclosure is intended to include all higher numerical limitations subsumed therein. Accordingly, Applicant reserves the right to amend the disclosure, including the claims, to expressly recite any sub-range subsumed within the ranges expressly recited herein.

The light-emitting device, the display device/apparatus, the electronic apparatus, the electronic equipment, a device of manufacturing the same, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in one or more embodiments. While one or more embodiments have been described with reference to the drawings, it will be understood by those of ordinary skill in the art that one or more suitable changes in form and details may be made therein without departing from the spirit and scope as defined by the appended claims and equivalents thereof.

What is claimed is:

1. A compound represented by Formula 1: 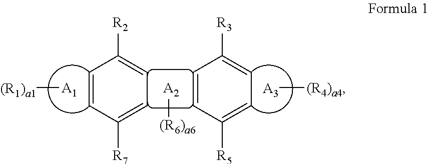
wherein, in Formula 1,
A₁ and A₃ are each independently hydrogen, deuterium, a cyano group, or a C₅ carbocyclic group,
A₂ is a C₄-C₆ carbocyclic group,
R₁ to R₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₈-C₆₀ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a4, and a6 are each independently an integer from 0 to 3, and when a1 is 2 or greater, then the two or more R₁(s) are identical to or different from each other, when a4 is 2 or greater, then the two or more R₄(s) are identical to or different from each other, and when a6 is 2 or greater, the two or more R₆(s) are identical to or different from each other, and
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ are each independently:
hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
wherein Formula 1 comprises a cyano group.

2. The compound of claim 1, wherein
A₁ and A₃ are each independently hydrogen, deuterium, a cyano group, or
a cyclopentene moiety.

3. The compound of claim 1, wherein
A₂ is a cyclobutane moiety or a bicyclo[2,2,0]hex-1(4)-ene moiety.

4. The compound of claim 1, wherein
the compound represented by Formula 1 is a symmetrical compound.

5. The compound of claim 1, wherein
the compound represented by Formula 1 is a compound represented by Formula 1-1: 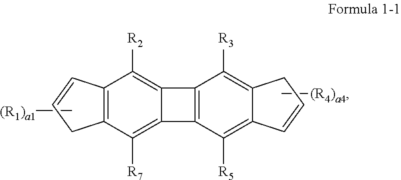
in Formula 1-1,
R₁ to R₅ and R₇, a1 and a4 being each the same as defined in Formula 1,
and Formula 1-1 comprising a cyano group.

6. The compound of claim 1, wherein
the compound represented by Formula 1 is a compound represented by Formula 1-2: 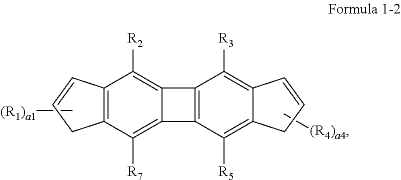
in Formula 1-2,
R₁ to R₅ and R₇, a1 and a4 being each the same as defined in Formula 1,
and Formula 1-2 comprising a cyano group.

7. The compound of claim 1, wherein
the compound represented by Formula 1 is a compound represented by Formula 1-3: 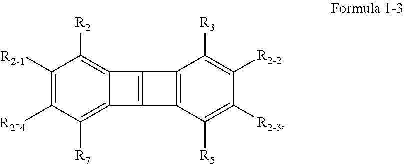
in Formula 1-3,
R₂₋₁ to R₂₋₄ being each independently hydrogen, deuterium, or a cyano group,
R₂, R₃, R₅ and R₇ being each the same as defined in Formula 1,
and Formula 1-3 comprising a cyano group.

8. The compound of claim 1, wherein
the compound represented by Formula 1 is a compound represented by Formula 1-4: 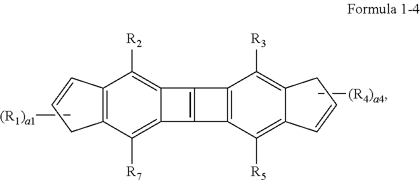
in Formula 1-4,
R₁ to R₅ and R₇, a1 and a4 being each the same as defined in Formula 1,
and Formula 1-4 comprising a cyano group.

9. The compound of claim 1, wherein
the compound represented by Formula 1 is a compound represented by Formula 1-5: 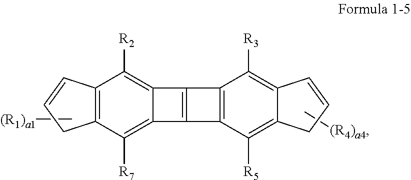
in Formula 1-5,
R₁ to R₅ and R₇, a1 and a4 being each the same as defined in Formula 1,
and Formula 1-5 comprising a cyano group.

10. The compound of claim 1, wherein
the compound represented by Formula 1 is any one selected from among compounds:

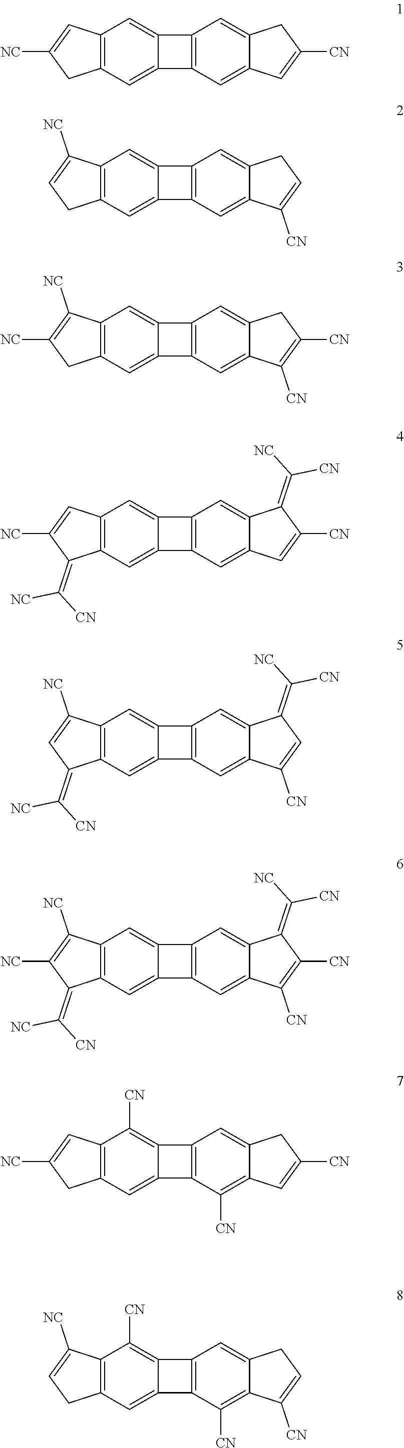

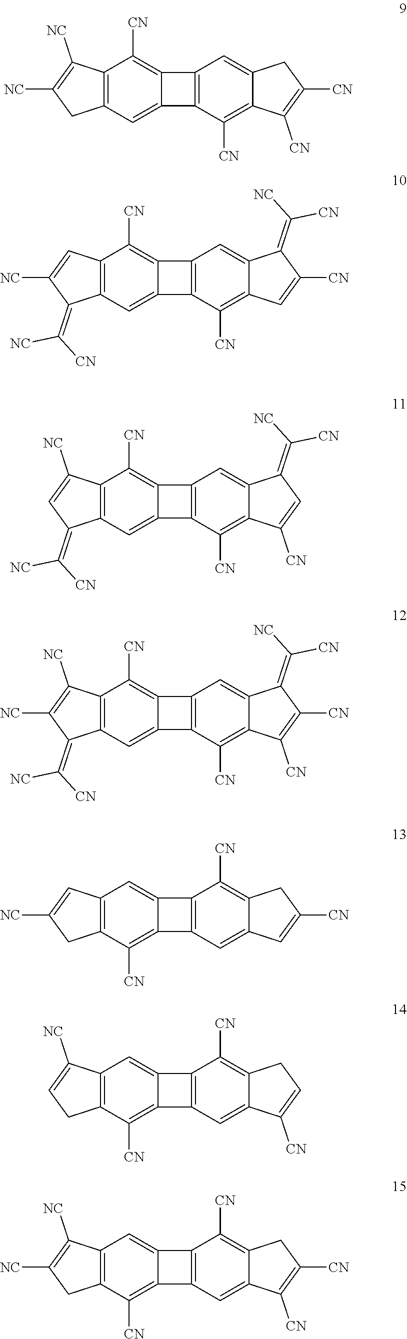

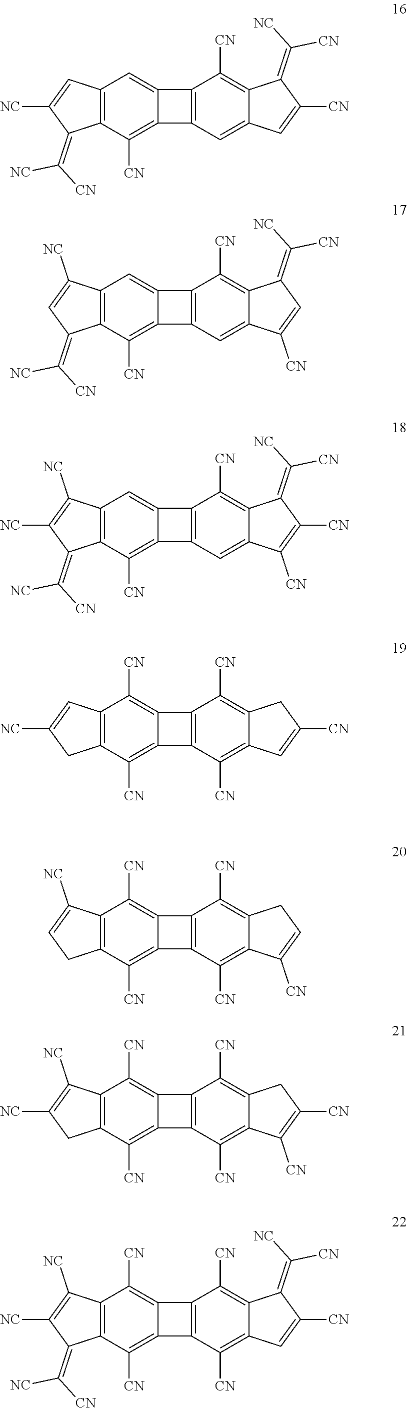

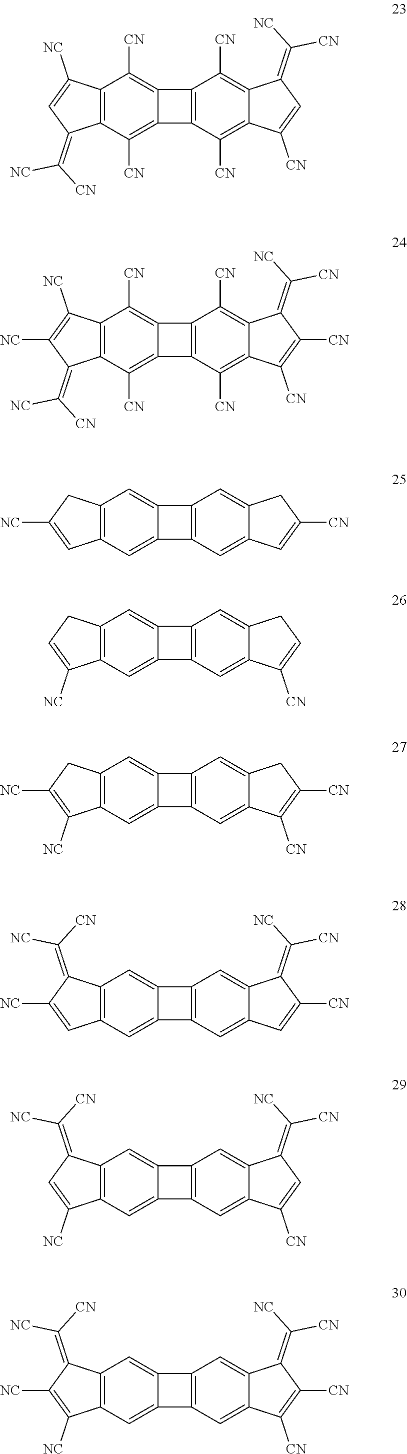

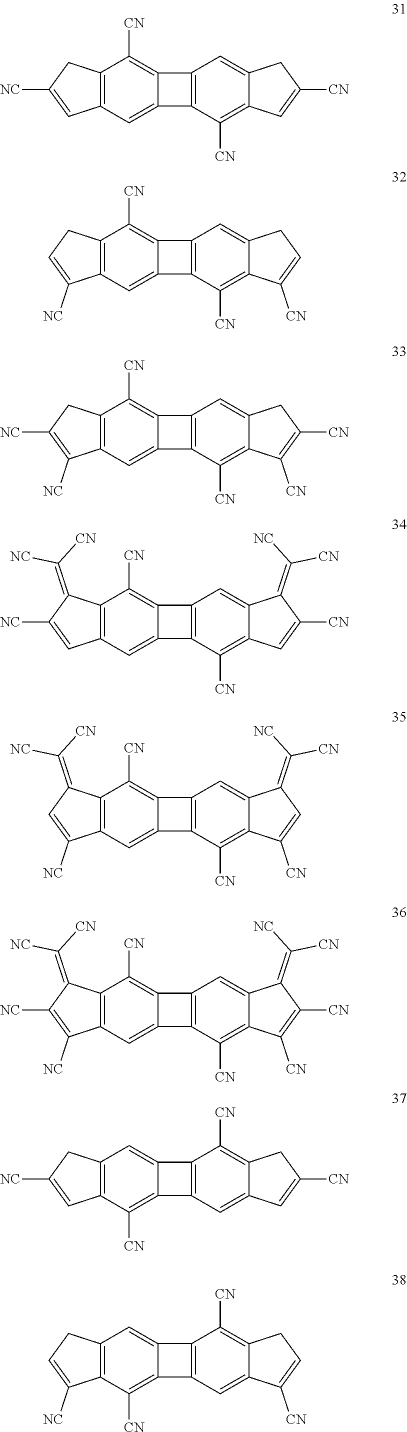

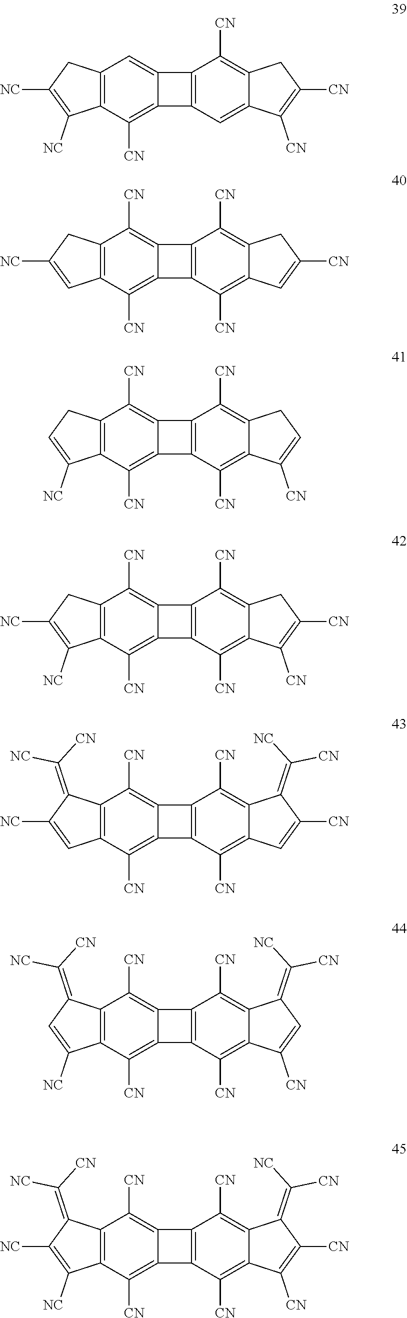

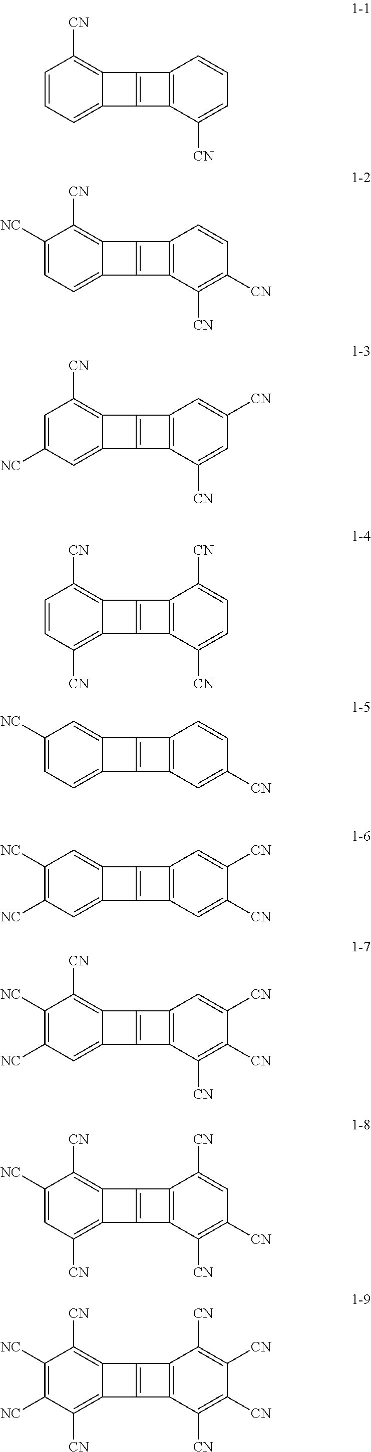

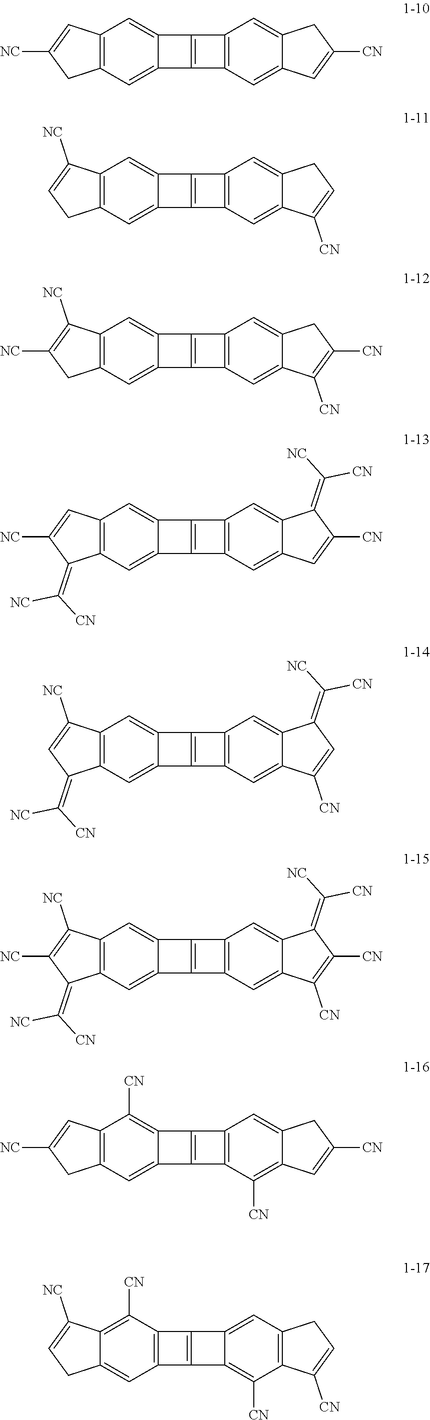

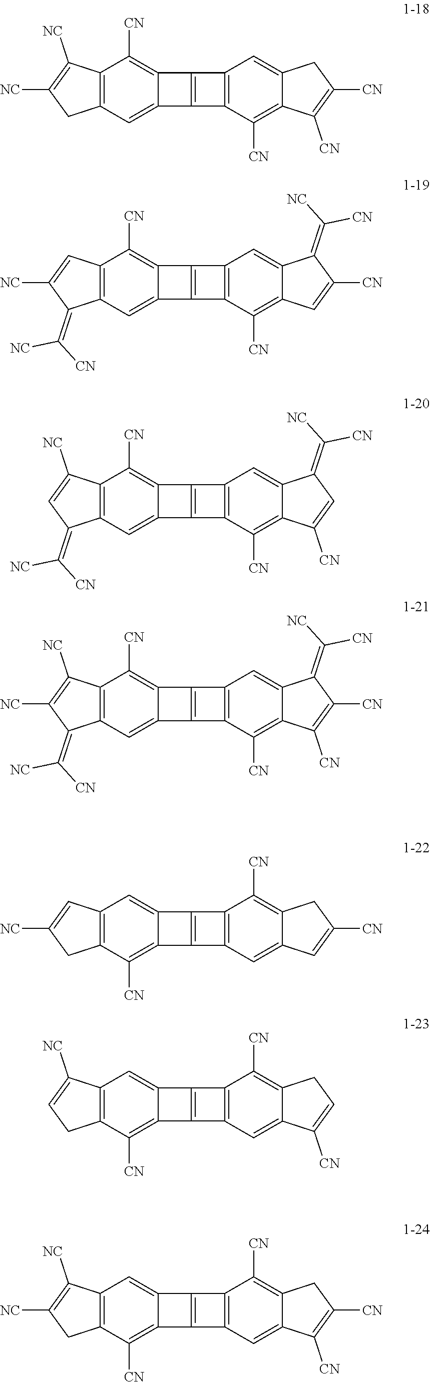

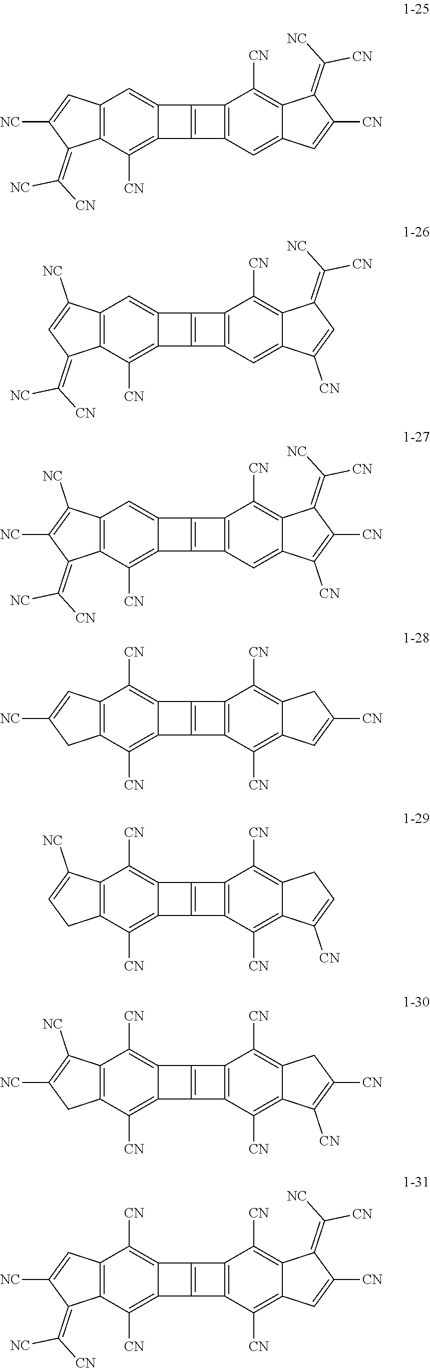

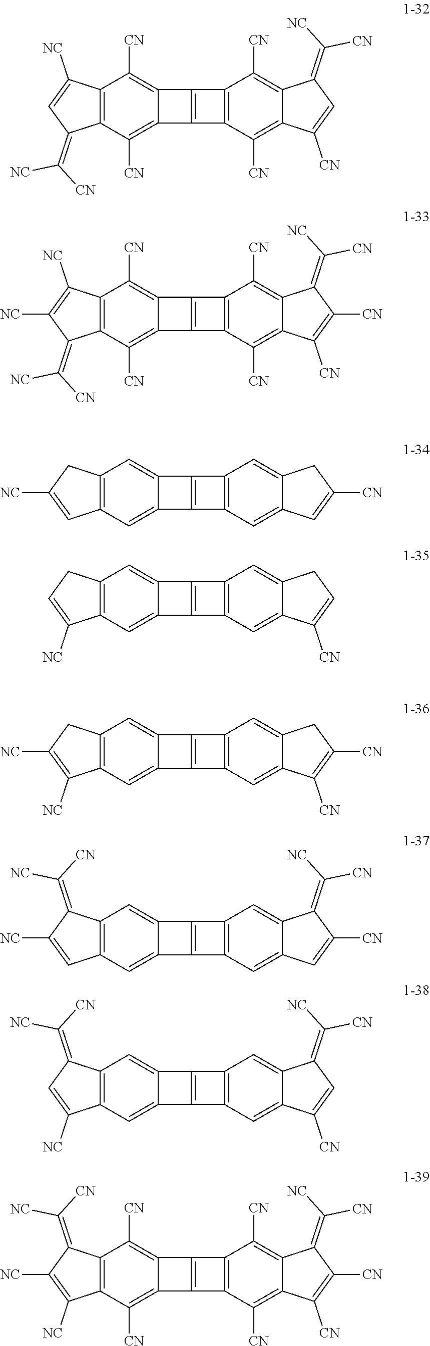

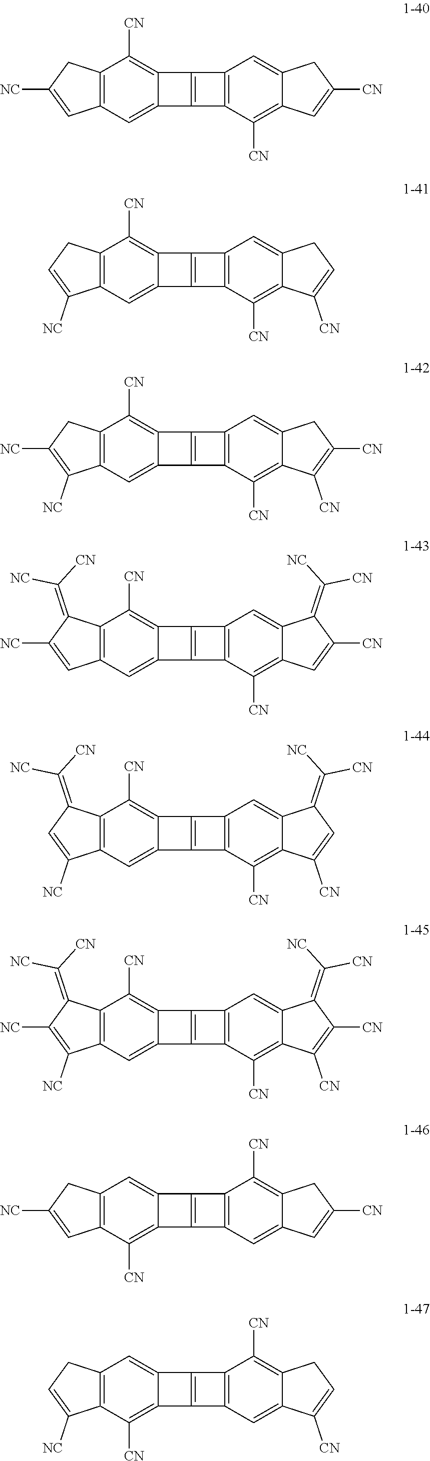

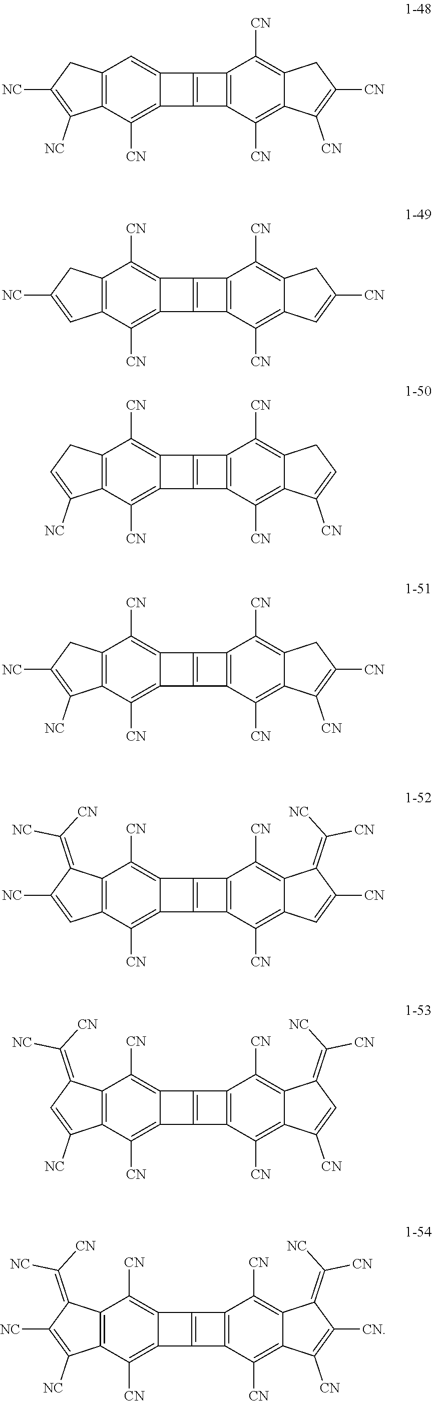

11. A light-emitting device comprising:
a first electrode;
a second electrode opposite to the first electrode; and
an interlayer between the first electrode and the second electrode and comprising an emission layer,
wherein the interlayer comprises the compound represented by Formula 1 of claim 1.

12. The light-emitting device of claim 11, wherein
the first electrode is an anode,
the second electrode is a cathode, and
the interlayer further comprises:
a hole transport region between the first electrode and the emission layer and comprising a hole injection layer; a hole transport layer, an electron blocking layer, an emission auxiliary layer, or any combination thereof; and/or
an electron transport region between the second electrode and the emission layer and comprising a hole blocking layer, an electron transport layer, an electron injection layer, or any combination thereof.

13. The light-emitting device of claim 12, wherein
the hole transport region comprises the compound represented by Formula 1.

14. The light-emitting device of claim 11, wherein
the emission layer comprises a first host, a second host, and a dopant, and
the dopant is a compound comprising a metal and a ligand, the ligand comprising an imidazole moiety.

15. The light-emitting device of claim 14, wherein
the first host is a hole-transporting host, and
the second host is an electron-transporting host.

16. The light-emitting device of claim 11, wherein
the emission layer comprises a red emission layer, a blue emission layer, and a green emission layer.

17. The light-emitting device of claim 11, wherein
the emission layer comprises m emission layers,
the interlayer further comprises m−1 charge generation layers respectively arranged between two adjacent emission layers among the m emission layers,
m is an integer of 2 or greater, and
any one layer selected from among the m−1 charge generation layers comprises the compound represented by Formula 1.

18. An electronic apparatus comprising the light-emitting device according to claim 11.

19. An electronic equipment comprising a light-emitting device, wherein:
the light-emitting device comprises:
a first electrode;
a second electrode opposite to the first electrode; and
an interlayer between the first electrode and the second electrode and comprising an emission layer,
wherein the interlayer comprises a compound represented by Formula 1: 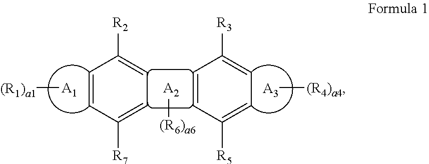
wherein, in Formula 1,
A₁ and A₃ are each independently hydrogen, deuterium, a cyano group, or a C₅ carbocyclic group,
A₂ is a C₄-C₆ carbocyclic group,
R₁ to R₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₈-C₆₀ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a4, and a6 are each independently an integer from 0 to 3, and when a1 is 2 or greater, then the two or more R₁(s) are identical to or different from each other, when a4 is 2 or greater, then the two or more R₄(s) are identical to or different from each other, and when a6 is 2 or greater, the two or more R₆(s) are identical to or different from each other, and
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(021), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ are each independently:
hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
wherein Formula 1 comprises a cyano group.

20. The electronic equipment of claim 19,
wherein the electronic equipment is at least one of a flat panel display, a curved display, a computer monitor, a medical monitor, a television, an advertisement board, an indoor light, an outdoor light, a signaling light, a head-up display, a fully transparent display, a partially transparent display, a flexible display, a rollable display, a foldable display, a stretchable display, a laser printer, a telephone, a mobile phone, a tablet, a phablet, a personal digital assistant, a wearable device, a laptop computer, a digital camera, a camcorder, a viewfinder, a microdisplay, a 3D display, a virtual reality display, an augmented reality display, a vehicle, a video wall comprising multiple displays tiled together, a theater screen, a stadium screen, a phototherapy device, or a signboard.
